SEQUENCE LISTING (4 sequences)

SEQ ID NO: 1 - RNA, 112 nt, Fusobacterium nucleatum
  misc_feature (110)..(112): This sequence contains a poly-C tail
aucuucggggcagggugaaauucccgaccggugguauaguccacgaaaguauuugcuuugauuuggugaaauuccaaaaccgacaguagagucuggaugagagaagauuccc

SEQ ID NO: 2 - RNA, 54 nt, Artificial Sequence
  Modified recombinant sequence derived from Fusobacterium nucleatum FMN Riboswitch 5′ sequence
ggaucuucggggcagggugaaauucccgaccggugguauaguccacgaaagcuu

SEQ ID NO: 3 - RNA, 56 nt, Artificial Sequence
  Modified recombinant sequence derived from Fusobacterium nucleatum FMN Riboswitch 3′ sequence
gcuuugauuuggugaaauuccaaaaccgacaguagagucuggaugagagaagauuc

SEQ ID NO: 4 - RNA, 176 nt, Thermotoga maritima
  misc_feature (174)..(176): This sequence contains a poly-C tail
ggccgacggaggcgcgcccgagaugaguaggcugucccaucaggggaggaaucggggacggcugaaaggcgagggcgccgaagggugcagaguuccucccgcucugcaugccuggggguauggggaauacccauaccacugucacggaggucucuccguggagagccgucgguccc

(19) United States
(12) Patent Application Publication
(10) Pub. No.: US 2011/0237549 A1
(43) Pub. Date: Sep. 29, 2011

(54) MODULATORS OF RNA RIBOSWITCHES

(75) Inventors: Dinshaw Patel, New York, NY (US); Alexander Serganov, New York, NY (US); Lili Huang, New York, NY (US)

(73) Assignee: MEMORIAL SLOAN KETTERING CANCER CENTER, New York, NY (US)

(21) Appl. No.: 12/934,408

(22) PCT Filed: Mar. 27, 2009
(86) PCT No.: PCT/US2009/001883

§ 371 Date: Jun. 14, 2011

Related U.S. Application Data
(60) Provisional application No. 61/064,817, filed on Mar. 27, 2008.
(60) Provisional application No. 61/129,005, filed on May 30, 2008.

(51) Int. Cl. A01N 57/16; G01N 33/53; C40B 60/12; C07F 9/6561; A01P 1/00; G06G 7/58

(52) U.S. Cl. 514/81; 436/501; 506/39; 544/244; 703/11

(57) ABSTRACT

Provided is a computer model generated from a data array, computer readable storage media encoded with the model, and computers comprising the model, wherein the model is derived from atomic structure coordinates of a FMN riboswitch or a lysine riboswitch. Further provided is a pharmacophore having a spatial arrangement of atoms defined by the binding pocket identified in the model, along with a compound defined by the pharmacophore. Also further provided are methods for rational drug design based on the atomic structure data, and compounds identified by the rational drug design process.

This application claims the benefit of U.S. Provisional Patent Application No. 61/064,817, filed Mar. 27, 2008, and U.S. Provisional Patent Application No. 61/129,005, filed May 30, 2008, the contents of which are hereby incorporated by reference in their entirety.

The present invention was made in part with support from a grant from the National Institutes of Health (NIGMS), grant R01 GM073618-18. Therefore, the government has certain rights in the invention.

### BACKGROUND OF THE INVENTIVE SUBJECT MATTER

1. Field of Inventive Subject Matter

The inventive subject matter relates to a computer model generated from a data array, computer readable storage media encoded with the model, and computers comprising the model, wherein the model is derived from atomic structure coordinates of an FMN riboswitch or a lysine riboswitch. The inventive subject matter further relates to a pharmacophore having a spatial arrangement of atoms defined by the binding pocket identified in the model, along with a compound defined by the pharmacophore. Finally, the inventive subject matter additionally relates to methods for rational drug design based on the atomic structure data, and to compounds identified by the rational drug design process.

2. Background

Riboswitches regulate gene expression at the mRNA level, typically occurring when untranslated segments undergo structural rearrangement upon binding to a ligand such as, in a common example, a cognate ligand which is the expression product of the gene which is transcribed to produce the mRNA. Alternate riboswitch ligands include other naturally occurring or artificially-created ligands. However, identifying such alternate ligands has traditionally been a random, time-consuming, and expensive process, generally involving trial-and-error experimentation and a degree of luck.

As bacterial resistance becomes ever more prevalent, and as evolutionary pressures diminish or destroy the effectiveness of many common antibiotic compositions, there is an increasingly great need to develop new compounds and compositions which can target bacteria in new ways that will provide antibiotic drugs in the future. Riboswitch modulation is one target for drug development, and improving the process by which alternate ligands are identified will significantly increase the speed and effectiveness of drug development, while likely reducing costs as well.

There are a variety of known riboswitch classes:
TPP riboswitch (also known as the THI-box), which binds thiamin pyrophosphate to regulate biosynthesis and transport, of thiamin and similar metabolites;
Cobalamin riboswitch (also known as the B12-element), which binds adenosylcobalamin to regulate cobalamin biosynthesis and transport of cobalamin and similar metabolites;
SAM riboswitches bind S-adenosyl methionine to regulate methionine and SAM biosynthesis and transport;
PreQ1 riboswitches bind pre-queuosine₁, to regulate genes involved in the synthesis or transport of this precursor to queuosine;
SAH riboswitches regulate genes involved in recycling S-adenosylhomocysteine, which is produced when S-adenosylmethionine is used as a methyl donor in methylation reactions;
Purine riboswitches binds purines and regulate purine metabolism and transport, especially guanine or adenine;
glmS riboswitches is self-cleaving when there is a sufficient concentration of glucosamine-6-phosphate;
Glycine riboswitch binds glycine to regulate glycine-metabolism genes, including the use of glycine as an energy source;
cyclic di-GMP riboswitches bind the signaling molecule cyclic di-GMP in order to regulate a variety of genes controlled by this second messenger;
FMN riboswitch binds flavin mononucleotide (FMN) to regulate riboflavin biosynthesis and transport;, and
Lysine riboswitch (also L-box) binds lysine to regulate lysine biosynthesis, catabolism, and transport.

Applicants herein have determined for the first time the structure of the FMN and lysine riboswitches, as well as the structure of each riboswitch bound to ligands of such riboswitches. It is expected that such structural data will permit rational drug design programs to identify alternate natural and/or synthetic ligands which will modulate the activity of riboswitches, and permit the synthesis of antibiotics of the future to target riboswitches of pathogenic bacteria.

### SUMMARY OF THE INVENTIVE SUBJECT MATTER

The inventive subject matter relates to a computer model generated from a data array, computer readable storage media encoded with the model, and computers comprising the model, wherein the model is derived from atomic structure coordinates of an FMN riboswitch or a lysine riboswitch. The inventive subject matter further relates to a pharmacophore having a spatial arrangement of atoms defined by the binding pocket identified in the model, along with a compound defined by the pharmacophore. Finally, the inventive subject matter additionally relates to methods for rational drug design based on the atomic structure data, and to compounds identified by the rational drug design process.

In particular, the inventive subject matter relates to a computer model of an FMN riboswitch generated from a data array comprising the atomic structure coordinates of an FMN riboswitch as set forth in any one of Tables 6-14, or a composite thereof.

The inventive subject matter further relates to a pharmacophore having a spatial arrangement of atoms defined by the binding pocket identified in the computer model described above.

The inventive subject matter also relates to an isolated compound, or a salt or solvate thereof, defined by the pharmacophore described above.

The inventive subject matter additionally relates to a method for identifying a compound that interacts with an FMN riboswitch, utilizing a 3-D molecular model of an FMN riboswitch as shown in any one of Tables 6-14, or a composite thereof, comprising:

(a) using said model in a method of rational drug design to identify candidate compounds that can bind an FMN riboswitch; and

(b) assaying the binding of a candidate compound identified in step (a) using a purified FMN riboswitch to thereby determine a binding characteristic, or lack thereof, of said compound.

Further, the inventive subject matter relates to a method of killing bacteria, comprising contacting the bacteria with a compound identified by the method described above.

In yet another aspect of the inventive subject matter, a method for identifying a compound that interacts with an FMN riboswitch, utilizing the crystal structure of an FMN riboswitch, comprises the steps of:

(a) modeling the FMN riboswitch with a test compound; and

(b) determining if the test compound interacts with the FMN riboswitch.

In addition, the inventive subject matter relates to a computer model of a lysine riboswitch generated from a data array comprising the atomic structure coordinates of a lysine riboswitch as set forth in any one of Tables 15-26, or a composite thereof.

The inventive subject matter further relates to a pharmacophore having a spatial arrangement of atoms defined by the binding pocket identified in the computer model described above.

The inventive subject matter also relates to an isolated compound, or a salt or solvate thereof, defined by the pharmacophore described above.

The inventive subject matter additionally relates to a method for identifying a compound that interacts with a lysine riboswitch, utilizing a 3-D molecular model of a lysine riboswitch as shown in any one of Tables 15-26, or a composite thereof, comprising:

(a) using said model in a method of rational drug design to identify candidate compounds that can bind a lysine riboswitch; and

(b) assaying the binding of a candidate compound identified in step (a) using a purified lysine riboswitch to thereby determine a binding characteristic, or lack thereof, of said compound.

Further, the inventive subject matter relates to a method of killing bacteria, comprising contacting the bacteria with a compound identified by the method described above.

In yet another aspect of the inventive subject matter, a method for identifying a compound that interacts with a lysine riboswitch, utilizing the crystal structure of a lysine riboswitch, comprises the steps of:

(a) modeling the lysine riboswitch with a test compound; and

(b) determining if the test compound interacts with the lysine riboswitch.

### BRIEF DESCRIPTION OF THE DRAWINGS

FIG. 1 is a series of drawings which depict the overall structure and tertiary interactions of the FMN-bound F. nucleatum riboswitch. FIG. 1a shows a homology-based schematic of the FMN riboswitch with key long-range interactions indicated by arrows. FIG. 1b shows a schematic of the riboswitch fold observed in the crystal structure of the complex. FIG. 1c shows overall riboswitch structure in a ribbon representation. FIG. 1d shows superposition of the P2-P6 and P3-P5 domains. FIGS. 1e and 1f, show distinct alignments of nucleotide triples in the P2-P6 and P3-P5 domains.

FIG. 2 is a series of drawings which depicts recognition of FMN by its riboswitch. a, A view of the junction with bound FMN. Magenta spheres depict cations in proximity to FMN. b, Details of riboswitch-FMN interactions. Hydrogen bond distances are shown with dashed lines. c, FMN-riboswitch interactions mediated by M1, assigned to an Mg⁺² cation. Magenta sticks depict coordination bonds. Mg⁺²-coordinated water molecules (blue spheres) are positioned on the basis of direct coordination bonds with phosphate and G33. d, Chemical structures (left panel) and fluorescent binding assay (right panel) of FMN and its analogues with the 112-nucleotide F. nucleatum riboswitch conducted in 2 mM MgCl₂ and 100 mM KCl. The dissociation constants (micromolar, mean 6 s.d.) are: FMN, 0.037560.0031; riboflavin, 39.8; lumiflavin, 59.0. e, Interaction of FMN with F. nucleatum riboswitch in 100 mM KCl as a function of variable Mg⁺² concentration. The dissociation constants (nanomolar, mean6s.d.) are: 0.5 mM MgCl₂, 406.0652.7; 1.0 mM MgCl₂, 211.762.1; 10.0 mM MgCl₂, 10.962.4.

FIG. 3 is a series of drawings which depicts the molecular interactions of FMN analogues with the riboswitch. a, All-atom superposition of the ligand-binding pocket for riboflavin-bound (blue and green) and FMN-bound (grey) riboswitches. Nucleotides in green are positioned within hydrogen-bond distances of the ribityl moiety of riboflavin. b, Superposition of riboflavin-bound (blue) and roseoflavin bound (pink and green) riboswitches, depicted as in a. c, Surface view inside of the FMN-bound riboswitch with large openings shown with red arrows.

FIG. 4 is a series of drawings which depicts the probing the FMN riboswitch tertiary structure. a, Probing of 59 32P-labelled 112-nucleotide RNA (0.5 mM) by V1 and T2 nucleases in the absence and presence of six fold excess of FMN. T1 and —OH designate RNase T1 and alkaline ladders, respectively. NR, no reaction. Weak and strong FMN-induced cleavage protections are shown in light and dark red, whereas weak and strong cleavage enhancements are shown in light and dark green. b, Projections of the nuclease cleavage reductions (light and dark red) and enhancement (green) on the riboswitch structure. c, Nucleotides potentially facilitating formation of the regulatory P1 helix. Hydrogen bonds in the G9NA104N(G33-C46) tetrad are indicted. FMN-induced cleavage reductions in the in-line probing assay 5 are in red.

FIG. 5. Predicted transcription attenuation mechanism for the R nucleatum impX FMN riboswitch. The anti-termination mechanism has been experimentally shown for the B. subtilis FMN riboswitch 4,5,30. The metabolite-sensing domain of the riboswitch folds in the presence of FMN thereby facilitating formation of the PI helix. Stabilization of the FMN-bound conformation of the sensing domain promotes formation of a transcription terminator in the downstream expression platform. Transcription of the gene, therefore, is prematurely terminated. In the absence of FMN, the PI helix is not formed and regions highlighted in green participate in the formation of an alternative anti-terminator hairpin conformation. In this case, transcription of the gene is not blocked. Nucleotides not present in the gene are shown in italics. FMN binds this 220-nucleotide RNA with the Kd 134.0±13.9 nM (mean±s.d.).

FIG. 6. Architecture and sequence conservation of the FMN riboswitch. a, Secondary structure schematic of the F nucleatum FMN riboswitch used in the study. The schematic is based on the models from refs. 5, 8, 17. Stem-loops are color-coded similarly to those in FIG. 1. Nucleotides conserved in >95% riboswitches among 183 sequences from Rfam data base 36 are in red color. Nucleotides participating in tertiary contacts are squared and connected by dashed lines. b, Three-dimensional structure of the F nucleatum FMN riboswitch (ribbon representation) in stereo view. Stem-loops and conserved nucleotides are color-coded as in panel (a). Violet and green spheres represent K+ and Mg²⁺ cations.

FIG. 7. Examples of tertiary structure elements of the F. nucleatum FMN riboswitch. a, Interactions between loop L6 and helix P2, stabilized by formation of A29-U94 and C30-G93 base pairs, and by stacking interactions between C13, U94, G93 and G84. b, Intercalation of A90 of loop L6 into T-loop motif of loop L2. c, All-atom superposition of T-loop motifs from loop L2 (nts 17-25) and tRNA (PDB ID lEHZ) (nts 53-61). The root mean square deviation (r.m.s.d.) is 2.1 angstrom. d and e, Continuous stacking interactions that provide connection between helices P6 and P5 (d), and P2 and P3 (e).

FIG. 8. Comparison of the P3-P5 domain of the FMN riboswitch and the H19-H20 domain (nucleotides 299-339) of the Thermus thermophilus 23S rRNA23 (PDB ID 2J03). a, All-atom superposition of the FMN riboswitch domain (yellow and cyan; nucleotides 33-37, 38-46, 62-73, and 75-84) and 23S rRNA (magenta; nucleotides 324-328, 330-338, 299-310, 311-320). R.m.s.d. is 1.48 A for the superposed residues. b, H19-H20 domain (magenta) in the context of ribosome. The RNA segments (various colors) and proteins (green) surrounding the domain are shown. The H19-H20 domain also shows similarity to the H25.1-H72 domain of the 23S rRNA (1FFX) from Haloarcula marismorti 37.

FIG. 9. The metabolite-sensing domain of the B. subtilis FMN riboswitch. a, The sequence of the 143-nucleotide fragment of B. subtilis riboswitch 5 is presented as a secondary structure schematic, which is based on the consensus secondary structure of the FMN riboswitches with small corrections from the three-dimensional structure of the F nucleatum riboswitch. b, Binding of FMN and its analogs to the 143-nucleotide B. subtilis FMN riboswitch. All measurements were performed using fluorescent assay. RNA was titrated against 6×10-8 M of FMN and 10-8 M of riboflavin or lumiflavin. Each experiment was performed-2-4 times in 50 mM Tris-HCl, pH 7.4, 100 mM KCl, 2 mM MgCl₂. The 172 nucleotide fragment of the B. subtilis lysine riboswitch 27 was used as a negative control.

FIG. 10. Comparison of the structures of the in-vitro-selected FMN-specific aptamer RNA and the FMN riboswitch. a, b, c, Ribbon (a) and surface (b) representations of the FMN-bound aptamer RNA28 and the FMN riboswitch (c). In contrast to the FMN riboswitch, FMN (red) in the complex with the aptamer intercalates into the distorted RNA helix, leaving a large opening along the short hydrophobic and long edges of the isoalloxazine ring. Major part of the ribityl-phosphate moiety does not bind RNA and protrudes outwards. d, e, Nucleotides located above (green) and in the same layer (grey and blue) with FMN in the complexes with the aptamer (d) and riboswitch (e) RNAs. FMN is stacked on the 09-027 base pair in the aptamer complex, and on the A8S in the riboswitch complex. Hydrogen bond distances are depicted with dashed lines. f, g, Nucleotides located below (cyan) and in the same layer (grey, blue and yellow) as FMN in the complexes with the aptamer (f) and riboswitch (g) RNAs. FMN is stacked on the (A2S-UI2)-010 triple and A48 in the aptamer and riboswitch complexes, respectively. Unlike the FMN riboswitch, the aptamer makes specific hydrogen bonds with the Hoogsteen edge of adenine.

FIG. 11. Mn2+ binding sites in the FMN riboswitch. The refined FMN riboswitch model (Mn2+ anomalous data) superposed with the anomalous map (pink) contoured at the 4.0 cr level. Mn2+ cations are shown as green spheres. Mn1 (˜11.5 cr level) is directly coordinated to the N7 position of G33 and the non-bridging phosphate oxygen of FMN and occupies the position of the FMN-bound Mg²⁺ cation M1 in the native structure of the FMN-riboswitch complex. Other Mn2+ cations also replace Mg²⁺ cations found in the native structure. Inset: the shortest distances from cations to FMN and RNA are indicated by dashed lines.

FIG. 12. Cs+ binding sites in the FMN riboswitch. The refined FMN riboswitch model (Cs+ anomalous data) superposed with the anomalous map (pink) contoured at the 3.5 (J level. Cs+ cations are shown as green spheres. Cs1 and Cs2 occupy positions not identified as cation-binding sites in the native FMN-riboswitch structure, while Cs3 and Cs4 replace K+ cations found in the native structure. Inset: the shortest distances from cations to heteroatoms of FMN and RNA are indicated by dashed lines.

FIG. 13. Effects of cations on the binding of FMN to the F. nucleatum FMN riboswitch. All measurements were performed using fluorescent assay and the 112-nucleotide riboswitch fragment. a, Dependence of the FMN binding upon cation concentration. Cations were titrated against RNA (2×10-7 M)-hg and (6×10-8 M) complex in 50 mM Tris-HCl, pH 7.4. Data were fitted to the equation (2) (see Methods). b, Effects of 2 mM divalent cations on the FMN-riboswitch interactions in the absence (top) and presence (bottom) of 100 mM KCl. c, Effects of 100 mM monovalent cations on the FMN riboswitch interactions in the presence of 2 mM MgCl. In both (b) and (c) RNA was titrated against 6×10-8 M of FMN in 50 mM Tris-HCI, pH 7.4, supplemented with different cations. Data were fitted to the equation (1) (see Methods).

FIG. 14. BaH binding sites in the FMN riboswitch. The refined FMN riboswitch model (Ba2+ anomalous data) superposed with the anomalous map (pink) contoured at the 3.0 cr level. Ba2+ cations are shown as purple spheres. Ba1 and Ba13 replace K+ cations; Ba1, Ba9, and Ba10 replace Mg²⁺ cations; and Ba2 replaces FMN-bound Mg²⁺ found in the native structure of the FMN-riboswitch complex. Other Ba2+ cations occupy positions not identified as cation-binding sites in the native FMN riboswitch structure. Ba2, Ba10, and Ba16 correspond to the positions of the Mn2+ cations; Ba5 and Ba13 correspond to the positions of Cs+ cations, while another Cs+ cation is positioned between Ba1 and Ba11. Ba1, Ba4, Ba5, Ba8, Ba11, Ba12, Ba14 and Ba15 are located close to [Ir(NH3)6]3+ groups. Inset: the shortest distances from Ba2+ cations to FMN and RNA are indicated by dashed lines.

FIG. 15. [Co(NH3)6]3+ binding sites in the FMN riboswitch. The refined FMN riboswitch model (Co anomalous data) superposed with the anomalous map (pink) contoured at the 4.0 cr level. [Co(NH3)6]3+ groups are shown as aquamarine spheres. All [Co(NH3)6]3+ groups, except Co2, are positioned similar to the [Ir(NH3)6]3+ groups. Co2 is located at the site of the FMN-bound Mg²⁺ cation M1. At this position, Co2 group forms close contacts with FMN and RNA, suggesting conformational adjustments in both RNA and phosphate moiety of FMN in order to accommodate bulky [Co(NH3)6]3+ group. These conformational changes, however, cannot be traced in the current structure due to the large proportion of the Mg²⁺-bound riboswitch form in the crystal, which may account for ˜90% of the complex (Mg²⁺ to [Co(NH3)6]3+ ratio was 8 to 1). Therefore, Co2 position has been refined as a Mg²⁺ cation. Inset: the shortest distances from cations to FMN and RNA are indicated by dashed lines.

FIG. 16. [Ir(NH3)6]3+ binding sites in the FMN riboswitch. The refined FMN riboswitch model with 11 [Ir(NH3)6]3+ groups shown in blue. 9 [Ir(NH3)6]3+ sites with high occupancy have been identified during structure phasing. 1r1 is located close to the K+ binding site M2 in the native structure of the FMN-riboswitch complex. 1r11 is occupied by Mg²⁺, and 1r7 site is occupied by a density assigned to a water molecule in the native structure.

FIG. 17. Superposition of the FMN riboswitch-ligand complexes. a, All-atom superposition of the FMN-bound (red) and riboflavin-bound (blue) riboswitches. R.m.s.d. is 0.62 angstrom. The riboflavin-bound riboswitch structure shows a small shift of P4 helix, indicated by an arrow, towards the core of the molecule. b, All-atom superposition of the roseoflavin-bound (green) and riboflavin-bound (blue) riboswitches. R.m.s.d. is 0.36 angstrom. All crystals used for comparison were grown with RNA prepared by annealing of the RNA oligonucleotides. Note that native structures of the FMN-riboswitch complexes prepared using transcribed RNA and RNA prepared from oligonucleotides are virtually identical.

FIG. 18. A hypothetical model of the FAD-riboswitch complex. a, Structural formula of FAD. b, A model showing the central region of the FMN riboswitch (dark green) with the bound FAD (light green, stick representation) and the corresponding region of the FMN-bound structure (grey). Nucleotides, whose conformations need to be adjusted to fit FAD into the pocket, are shown in stick representations. To build a model, FAD was inserted into the pocket on the basis of the bound FMN in the FMN-riboswitch structure using TURBO-FRODO. The model was then refined using REFMAC and experimental data, collected from the riboswitch crystals grown in the presence of FAD. The experimental data showed electron density map only for the FMN moiety of FAD, and did not show any density for the remaining part of the molecule. Therefore, the placement of the adenine moiety in the model is not based on the experimental data. It is likely that the labile phosphoanhydride bond of FAD was hydrolyzed to produce FMN during crystallization. The resulting map, when refined with FMN, did not show significant deviations from the map of the FMN-bound structure.

FIG. 19. Surface views of the FMN riboswitch bound to FMN. FMN and K+ and Mg²⁺ cations, proximate to FMN, are shown in red, violet and magenta colors. a, Front view, similar to the view on Fig. b, Back view.

FIG. 20. Footprinting results for the F. nucleatum FMN riboswitch. The figure summarizes data from FIG. 4a and complements FIG. 4b. (a) Summary of VI nuclease footprinting experiments performed on the F nucleatum riboswitch projected on the secondary structure schematic (left) and the three-dimensional structure (right). Nucleotides participating in tertiary contacts are squared and connected by dashed lines. (b) Summary of T2 nuclease footprinting experiments shown as in panel (a).

FIG. 21. In-line probing of FMN riboswitch. Projection of in-line probing data for the B. subtilis riboswitch from ref. 5 on the F nucleatum riboswitch secondary structure schematic (top) and the three-dimensional structure (bottom).

FIG. 22. Point mutations in the sensing domain of the FMN riboswitch causing resistance to roseoflavin and deregulation of the gene expression. Projection of known point mutations in the sensing domains of B. subtilis 4, 38-40, B. amyloliquefaciens 40, Lactococcus lactis 29, Leuconostoc mesenteroides 15, and Propionibacterium freudenreichii 15 FMN riboswitches on the sequence (top) and structure (bottom) of the F nucleatum FMN riboswitch. Mutations causing the derepression of the riboswitch-controlled gene expression and resistance to roseoflavin are indicated by arrows and pink color. Nucleotides conserved in >95% riboswitches presented in Rfam36 data base are in red color.

FIG. 23. Interpretation of the electron density maps. a, b, Experimental electron density map (contoured at the 1 cr level) around the FMN binding pocket (a) and FMN (b) shown with the refined riboswitch model. The map was calculated using 3.0 A [Ir(NH3)6]3+ MAD data. c, d, 3.0 A omit Fo-Fc maps (2 cr level) calculated without riboflavin (c) and roseoflavin (d) and shown with the refined ligand models. e-g, Refined 2.95 A2Fo-Fc electron density map (blue, 1 cr level; red, 2 cr level) around FMN from the top (e), front (f) and side (g) views. FMN can undergo reversible redox interconversion between oxidized (FMN), semiquinone (FMNHe) and reduced (FMNH2) states. In the semiquinone and reduced forms, the isoalloxasine ring system is slightly bent along the N5-NIO axis 41. In the FMN riboswitch crystals, FMN ring system is planar consistent with the presence of the oxidized FMN. h, Refined 3.0 A 2Fo-Fc electron density map (1 cr level) in the region of the conformational changes observed in the riboflavin-riboswitch complex.

FIG. 24. Overall structure and long-range tertiary interactions of the lysine-bound T. maritima riboswitch. a, Schematic of the riboswitch fold observed in the crystal structure of the complex. The bound lysine is in red. The RNA domains are depicted in colors used for subsequent figures. Base specific tertiary contacts and long-range stacking interactions are shown as thin green and thick blue dashed lines, respectively. Nucleotides invariant in known lysine riboswitches are boxed. b, c, Overall lysine riboswitch structure in a ribbon representation showing front (b) and rotated by ,60u (c) views. d, The L2-L3 kissing loop interaction is formed by six base pairs, supplemented by interstrand stacking interactions between A42 and C95, G43 and U94, and G44 and G101. Hydrogen bonds between interstrand base pairs and orthogonally aligned G43 and U94 bases are depicted by dashed lines. e, The L4-loop-P2-helix interaction formed by an insertion of the A126-A127-A129 stack of L4 into the RNA groove of P2 distorted by noncanonical base pairs.

FIG. 25. Structure and interactions in the junctional region of the lysine riboswitch. a, Stereo view of the junction with bound lysine. Green sphere depicts a K⁺¹ cation. b, Details of riboswitch lysine interactions. Lysine is positioned within the omit Fo2Fc electron density map contoured at 3.5s level. Water molecules are shown as light blue spheres. K⁺¹ cation coordination and hydrogen bonds are depicted by dashed lines. c, Direct and water-mediated interactions involving e-ammonium group of lysine. d, e, Interactions in the top (d) and middle (e) junctional layers.

FIG. 26. Interactions of lysine analogues with the riboswitch. a, Chemical structures of lysine and its analogues. b, Conformation and interactions of lysine analogues with the riboswitch. Lysine (red) and analogues (cyan) are superposed. Interactions between riboswitch and lysine analogues should be compared with lysine recognition in FIG. 2b. c, Cross-section through the surface view of the lysine-binding pocket showing the opening next to the carboxyl group of lysine (red arrow) and the free space next to the C4 atom (blue arrow). d, Cross-section through the homoarginine-riboswitch complex showing the opening (red arrow) next to the guanidinium group.

FIG. 27. Probing lysine riboswitch tertiary structure. a, Primer extension analysis at various K⁺¹ concentrations. 32P-labelled oligonucleotide was annealed to the 39 end of 265-nucleotide T. maritima RNA (1 mM) and extended by reverse transcriptase in the presence of 2 mM MgCl₂, at indicated concentrations of monovalent cations (in mM), and with or without a tenfold excess of lysine over RNA. b, Lysine binding affinity measured by equilibrium dialysis for riboswitches from T. maritima (top) and B. subtilis (bottom). Mg⁺², K⁺¹ and Na⁺¹ concentrations are 20, 100 and 100 mM, respectively. The dissociation constants (mean6s.d., mM; n52-4) are: T. maritima, 0.1060.03 (Mg⁺²1K⁺¹), 4.1460.67 (Mg⁺²Na⁺¹), 15.9360.09 (Mg⁺²); B. subtilis, 2.9560.30 (Mg⁺²K⁺¹). c, The apparent ligand concentration at which reverse transcriptase pausing is half-maximally attained (P50; n52) in the primer extension experiments. HArg, L-homoarginine; IEL, iminoethyl-L-lysine; Lys, L-lysine; Oxa, L-4-oxalysine. d, In-line probing of 59 32P-labelled T. maritima 174-nucleotide RNA (1.6 nM) in the absence and presence of lysine (1.1 mM). T1 and 2OH designate RNase T1 and alkaline ladders, respectively. NR, no reaction. Strong and weak cleavage reductions are shown in red and pink colors, respectively. e, f, Probing of 174-nucleotide RNA (1 mM) by V1 and T2 nucleases with or without a tenfold excess of lysine. Weak and strong cleavage enhancements are shown in light and dark green, respectively, in e. g, Strong lysine-induced cleavage reductions are color coded in the riboswitch structure. Light orange, green and red are reductions identified in ref. 1 using B. subtilis RNA with a short P1 helix, overlapping reductions of the present study and in ref. 1, and extra reductions found in the present study, respectively.

FIG. 28. Alternative conformations of the T. maritima lysine riboswitch and mechanism of lysine-induced transcription termination. a, The metabolite-sensing domain of the riboswitch folds in the presence of lysine and facilitates formation of the PI helix and a transcription terminator in the downstream expression platform. Transcription of the gene, therefore, is prematurely terminated. b, In the absence of lysine, the PI helix does not form and regions highlighted in green participate in the formation of an alternative anti-terminator hairpin conformation. In this case, transcription of the gene is not blocked. Nucleotides shown in italics have been added to the 265-nt RNA fragment used in primer extension experiments. Nucleotide numbering is consistent with the numbering used for the shorter RNA fragment.

FIG. 29. Architecture and sequence conservation of the lysine riboswitch. a-b, Secondary structure schematics of the 172-nt B. subtilis (a) and the 174-nt T maritima (b) riboswitches used in the study. The schematics are based on the models from refs. 1-3,33. Stem-loops are color-coded similarly to those in FIG. 1. Red and blue nucleotides indicate invariant and conserved (>75 identity) nucleotides present in the T maritima riboswitch and III other lysine riboswitches from the Rfam database 34. The conservation analysis of the P2a-L2 tum and the L2-L3 kissing loops may not be accurate due to the length variations and possible sequence misalignments. Important long-range tertiary contacts are shown by dashed lines (blue lines for stacking and green lines for pairing). c, Schematics of the structure of the T maritima lysine riboswitch presented according with ref. 35. Invariant and conserved nucleotides, as well as color codes for the stem-loops are depicted as in (b). d, Overall lysine riboswitch structure in a ribbon representation. Invariant and conserved nucleotides, as well as color codes for the stem-loops are depicted as in (b).

FIG. 30. Structures and sequences of the RNA motifs from the T. maritima lysine riboswitch. a, Loop E motif. Left panel, sequences from the lysine riboswitch and 5S rRNA (PDB ID code IJJ2). Right panel, all-atom superposition of the loop E motif from the riboswitch (cyan) and 5S rRNA (brown). b, P2a-L2 turn from the lysine riboswitch, replacing the kink-turn motif found in other lysine riboswitches. Left panel: schematic representation of the turn. Watson-Crick and non-canonical base-pairs are shown by lines and circles, respectively. Stacking interactions are indicated by black rectangles. RNA backbone is depicted by solid colored lines. Right panel: the structure of the turn. Dashed lines depict hydrogen bonds. Note the syn conformation of C41. c, Kink-turn motifkt-7 from 16S rRNA24. Left panel: schematic representation of the motif. Right panel: superposition of the kt-7 turn (residues 77-82 and 93-99 in brown) (PDB ID code IJJ2) and the lysine riboswitch P2a-L2 turn (residues 37-42 in cyan and 50-56 in red). R.m.s.d. is 3.9 angstrom. Despite some similarities in the overall folds of these turns, the turn from the T maritima lysine riboswitch does not have the typical features of the canonical kink-turn motifs. d, A turn from the P4-P6 domain of group I introns (PDB ID code 1JID)36. Left panel: schematic representation of the motif. Right panel: superposition of the P4-P6 turn (residues 119-123, 126, and 196-202, in green) and the P2a-L2 turn (residues as in (c)). R.m.s.d. is 3.7 A.

FIG. 31. Structural details of the L2-L3 loop interaction. a, Hydrogen bonding of G43 and U94 with base pairs of the inter-loop helix. G43 and U94 are oriented perpendicular to other bases in the loops, positioned inwards toward the RNA groove, and involved in hydrogen bonding with the G45-G46-A47 and C96-U97-C98 segments, respectively. The G431U94 element, absent in other kissing loop complexes, zippers up the entire motif and likely contributes to the overall stability of the riboswitch conformation. band c, Side and top views of the all-atom superposition of the L2-L3 kissing loops from the lysine riboswitch (residues 44-49 in cyan and 95-100 in orange) and the kissing loop complex from the HIV-1 dimerization initiation site (DIS) (residues 10-15, chains C and D in grey) (PDB ID code 1K9W)37. R.m.s.d. of the superposed structures is 1.5 angstrom. The lysine riboswitch loops are closed by the non-canonical A42•A50 base pair, which is part of the P2a-L2 tum, and the U93•G101 base pair. Six canonical base pairs forming the intra-loop helix are superposed well between both structures. In the DIS structure, however, the residues corresponding to the G431U94 element are oriented outwards.

FIG. 32. Structural details of junction organization. a, Na+- and water-mediated interactions that contribute to the anchoring of G80 and the stabilization of the segments from helices PI (in magenta) and P3 (in orange) adjacent to the junction. b, Formation of a purine quartet by interactions between the conserved non-canonical G11-G163 and G141-A162 pairs. Note the syn conformation of G11.

FIG. 33. K+ binding site in lysine riboswitch. The refined lysine riboswitch structure (2.9 AK+ anomalous data) superposed with the anomalous map (pink) contoured at the 4.5 cr level and at the 3.5 cr level in the zoomed-in view. The zoomed-in view is slightly rotated with respect to the overall view. Na+ cations and the lysine-bound K+ cation are shown as violet and green spheres, respectively. Two more K+ cations found in the 1.9 A native structure are not shown in this figure. These K+ cations have weak peaks (the 3.1 and 3.5 cr levels, practically noise levels) on the 2.9 A anomalous map and do not have positive electron density on the 2.9 A2Fo-Fc map.

FIG. 34. Cs+ binding sites in the lysine riboswitch. The refined lysine riboswitch structure (Cs+ anomalous data) superposed with anomalous map (pink) contoured at the 4.5 0: level. Cs+ and Na+ cations are shown as light green and violet spheres, respectively. Two cesium cations, CSj+ (˜5 CJ level) and CS2+ (˜5.5 CJ level), have been identified in the map. CSj+ replaces the K+ cation coordinated with the bound lysine. CS2+ binds in a site that, in the high resolution native structure, is occupied by an elongated electron density, modeled as a small segment of a largely unstructured PEG molecule.

FIG. 35. TI+ binding sites in the lysine riboswitch. The refined lysine riboswitch model (TI+ anomalous data) superposed with the anomalous map (light pink) contoured at the 3.5 q level and the 30 (J level in the zoomed-in view. TI+ and Na+ cations are shown as brown and violet spheres, respectively. The water molecule is in light blue. Ten TI+cations, TIl+ to TIlQ+ (˜4-42 (J level) have been identified in the map. T19+ is split into two sites. TIl+, corresponding to the strongest anomalous peak (˜42 (J level), replaces the lysine-coordinated K+ cation. T12+ and Tb+ replace Na+ cations, Th+ and T18+ replace water molecules, and TIlQ+ replaces the segment of a PEG molecule, while the other TI+ cations bind new sites typically in close proximity to the water molecules identified in the native high-resolution structure.

FIG. 36. Mn2+ binding sites in the lysine riboswitch. The refined lysine riboswitch model (Mn2+ anomalous data) superposed with the anomalous map (pink) contoured at the 4.0 a: level. Mn2+, K+ and Na+ cations are shown as cyan, green and violet spheres, respectively. To compensate for the possible chelating effect of Na-citrate in the crystallization solution, Mn2+ concentration was increased up to 50 mM during soaking. Two manganese cations, Mn12+ (˜5 cr level) and Mn22+ (˜3 cr level, practically a noise level), have been identified in the map. Mn12+ is coordinated with the non-bridging phosphate oxygen and occupies the position of a water molecule in the high resolution native structure. Due to the lower resolution of this structure (2.7 A), Mn12+-coordinated water molecules may not be seen in the map. Mn22+ binds to the Sf-triphosphate moiety. No anomalous signal has been observed at the position occupied by the lysine-bound K+.

FIG. 37. Comparison of the lysine riboswitch structures obtained from the crystals grown in the absence and presence of Mg²⁺. All-atom superposition of the lysine riboswitch structures crystallized in the presence (red) and absence (blue) of Mg²⁺. R.m.s.d. is 0.33 A.

FIG. 38. Surface views of the riboswitch bound to lysine. a, Lysine (red color) shown inside of the binding pocket. The view is similar to the view in FIG. 2c. b, The view of the opening next to the carboxylate group of bound lysine; c, The view of the opening next to the £-ammonium group of the bound lysine. Positions of the lysine-bound K+ cation and water molecules are indicated by green and blue spheres, respectively. Note that the colored spheres representing the cation and water molecules should be distinguished from their shadows.

FIG. 39. Interactions of lysine and its analogs with the T. maritima lysine riboswitch studied by primer extension. a and b, Representative gels of riboswitch titration by lysine (a) and AEC (b). Reverse transcriptase (RT) pauses three nucleotides prior to the j unction at A169 in the context of the full-length riboswitch if the riboswitch-binding ligand is present in the reaction mixture. c, Representative titration experiments used for calculations of the ligand concentration (FIG. 4c) at which RT pausing is half-maximally attained. The experimental data points for lysine and AEC are from panels (a) and (b). The data were fitted to equation (1), which is based on the equation describing a bimolecular equilibrium, by the nonlinear least squares analysis. In the equation

$$θ = \frac{( {L}_{0} + {N}_{0} + {P}_{50} ) - \sqrt{{( {L}_{0} + {N}_{0} + {P}_{50} )}^{2} - 4 {L}_{0} {N}_{0}}}{2 {N}_{0}}$$

θ is the fraction of RT pausing, No and Lo are RNA and ligand concentrations, respectively, and P50 is the apparent ligand concentration at which RT pausing is half-maximally attained. The P50 value for the lysine-induced RT pausing 5.50±0.53/−1M (mean±s.d.) (n=2) is similar to the Kd value 4.03±0.34/−1M (mean±s.d.) (n=2) determined by the equilibrium dialysis assay. The relative differences between the P50 values for lysine and its analogs are in overall agreement with the relative differences obtained from the Kd values for lysine and its analogs determined for the B. subtilis lysine riboswitch in ref. 5.

FIG. 40. Footprinting of the T. maritima lysine riboswitch using in-line probing (a) and nuclease footprinting (b). Cleavages (in-line probing only) and lysine-induced cleavage reductions and enhancements are shown in the three-dimensional structures according to the color coding of the corresponding schematics. The assessment of cleavages near the RNA termini was not reliable and, therefore, was excluded from analysis (blue nucleotides). Most of the nucleotides demonstrating constant scission in the in-line probing assay correspond to the looped out nucleotides in the three-dimensional structure. Cleavage reductions, not identified in the study on the longer B. subtilis RNA with the shorter PI helix 1 (e.g., nucleotides 22-26, 40-41, 113-116), become apparent when lysine is present in large excess over RNA. Nuclease VI cleavage enhancements within the P2a helix can be explained by an increase in the helix rigidity of the lysine-bound riboswitch and by the increased scission of the most accessible RNA regions that results from the hindrance of other cleavage sites upon lysine binding. Note that C79, G80 and G12 can be stacked in the free riboswitch, as suggested by the VI cleavages. Such stacking may contribute to the correct orientation of these nucleotides for interactions upon lysine binding.

FIG. 41. A hypothetical model of lysine and K+ binding to the riboswitch junction. The lysine could enter the partially pre-formed binding pocket through an opening, which would otherwise be occupied in the bound state by K+ and G12. The lysine could then form specific hydrogen bonds with G114 and make multiple interactions that involve its £-ammonium group. The pocket could close via K+-mediated interactions and base-pairing of G12 and G11, that is next extended to the adjacent segment of the PI helix. Red lines designate important lysine-RNA interactions.

FIG. 42. Lysine riboswitch structure in the free state. a, All-atom superposition of the lysine riboswitch structures from crystals grown in the presence (red) and absence (blue) of lysine. R.m.s.d. is 0.35 angstrom. b, Refined 3.1 A 2Fo-Fc electron density map (contoured at 1 a level) around the lysine-binding pocket in the lysine-free riboswitch structure. The view is similar to that of FIG. 44a. Both lysine and the K+ cation are missing in the structure.

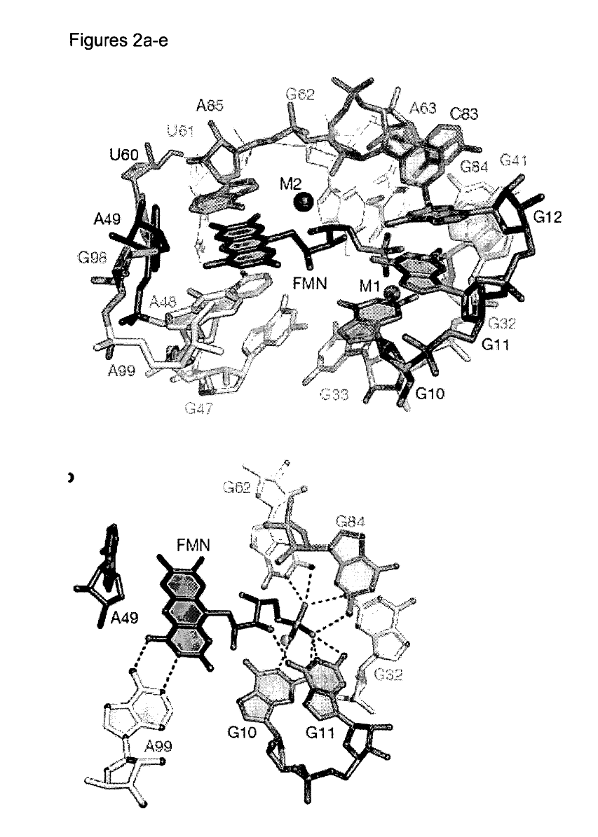

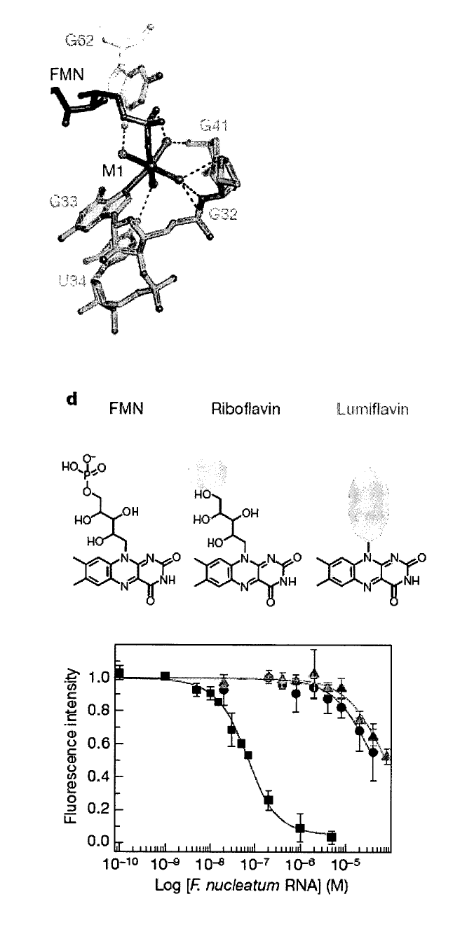

FIG. 43. Mutations in the sensing domain of the lysine riboswitch causing resistance to antibiotics and deregulation of the gene expression. Projection of known point mutations in the sensing domains of B. subtilis and E. coli lysine riboswitches on the sequence (a) and structure (b) of the T maritima lysine riboswitch. Mutations causing the derepression of riboswitches and resistance to AEC and L-4-oxalysine are indicated by pink arrows. Sequence conservation of the lysine riboswitch is indicated as in Supplementary FIG. 2. The point mutation A67C (B. subtilis numbering) in the kink-tum motif of B. subtilis riboswitch, mutations in the L2 and L3 100ps21, the long deletion of P2/J2-3/P327 and duplication within the PI helix 28 are not shown.

FIG. 44. Interpretation of certain regions of the electron density maps. a, Experimental electron density map (contoured at the 1 ( ) level) around the lysine binding pocket shown with the refined riboswitch model. The map was calculated using 2.4 A [Ir(NH3)6]3+ MAD data. b, 16 [Ir(NH3)6]3+ sites (2 sites are split) shown with the refined riboswitch model. c, Refined 1.9 A2Fo-Fc electron density map (contoured at the 1 ( ) level) around lysine and the K+ cation. Coordination bonds and distances are indicated. Water molecules are shown as pink spheres. d, Two Na+ cations interacting with the same 06 atom of guanine and sharing two water molecules. The map is as in (c).

### DETAILED DESCRIPTION OF THE INVENTIVE SUBJECT MATTER

### Definitions

The term “riboswitch” refers to a part of an mRNA molecule that can directly bind a target molecule, and whose binding of the target affects the activity of the gene that produces the mRNA molecule. Thus, an mRNA molecule that contains a riboswitch is directly involved in regulating the activity of the DNA sequence from which it is transcribed.

The term “effecting” refers to the process of producing an effect on biological activity, function, health, or condition of an organism in which such biological activity, function, health, or condition is maintained, enhanced, diminished, or treated in a manner which is consistent with the general health and well-being of the organism.

The term “modulating” as used herein refers to the process of increasing or decreasing activity.

The term “enhancing” the biological activity, function, health, or condition of an organism refers to the process of augmenting, fortifying, strengthening, or improving.

The term “isomers” refer to different compounds that have the same molecular formula. “Stereoisomers” are isomers that differ only in the way the atoms are arranged in space. “Enantiomers” are a pair of stereoisomers that are non-superimposable mirror images of each other. “Diastereoisomers” are stereoisomers which are not mirror images of each other. “Racemic mixture” means a mixture containing equal parts of individual enantiomers. “Non-racemic mixture” is a mixture containing unequal parts of individual enantiomers or stereoisomers.

The term “pharmaceutically acceptable salt, ester, or solvate” refers to a salt, ester, or solvate of a subject compound which possesses the desired pharmacological activity and which is neither biologically nor otherwise undesirable. A salt, ester, or solvate can be formed with inorganic acids such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, naphthylate, 2-naphthalenesulfonate, nicotinate, oxalate, sulfate, thiocyanate, tosylate and undecanoate. Examples of base salts, esters, or solvates include ammonium salts; alkali metal salts, such as sodium and potassium salts; alkaline earth metal salts, such as calcium and magnesium salts; salts with organic bases, such as dicyclohexylamine salts; N-methyl-D-glucamine; and salts with amino acids, such as arginine, lysine, and so forth. Also, the basic nitrogen-containing groups can be quarternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; aralkyl halides, such as benzyl and phenethyl bromides; and others. Water or oil-soluble or dispersible products are thereby obtained.

### Modulation of Riboswitches

Most clinical antibacterial compounds target one of a small number of cellular processes. Because bacteria have well-developed resistance mechanisms to protect these essential processes, it is very useful to discover and validate new targets. Riboswitches can be an effective target for controlling gene expression in natural organisms.

One potentially vulnerable bacterial process is the regulation of gene expression by riboswitches. Thus, an mRNA molecule that contains a riboswitch is directly involved in regulating its own activity, depending on the presence or absence of its target molecule. Typically found in the 5′ untranslated regions (5′ UTRs) of certain bacterial mRNAs, riboswitches form structured receptors (or aptamers) that bind fundamental metabolites. In most cases, ligand binding regulates the expression of genes involved in the synthesis and/or transport of the bound metabolite.

Because the biochemical pathways that are regulated by riboswitches may be essential for bacterial survival, modulation, in most cases down-regulation, of these pathways through riboswitch targeting is expected to be lethal.

Riboswitches are conceptually divided into two parts: an aptamer and an expression platform. The aptamer directly binds the small molecule, and the expression platform undergoes structural changes in response to the changes in the aptamer. The effect on the expression platform is what modulates or regulates gene expression.

Expression platforms typically turn off gene expression in response to the ligand, but some turn it on. Exemplary expression platforms may include:
The formation of rho-independent transcription termination hairpins
Folding in such a way as to sequester the ribosome-binding site, thereby blocking translation
Self-cleavage (i.e. the riboswitch contains a ribozyme that cleaves itself in the presence of sufficient concentrations of its metabolite)
Folding in such a way as to affect the splicing of the pre-mRNA.

The FMN riboswitch (also known as the RFN-element) binds flavin mononucleotide (FMN) to regulate riboflavin biosynthesis and transport. The FMN riboswitch is a highly conserved RNA element that is found frequently in the 5′-untranslated regions of prokaryotic mRNAs that encode for flavin mononucleotide (FMN) biosynthesis and transport proteins. This element is a metabolite-dependent riboswitch that directly binds FMN in the absence of proteins. In Bacillus subtilis, the riboswitch most likely controls gene expression by causing premature transcription termination within the 5′ untranslated region of the rib DEAHT operon and precluding access to the ribosome-binding site of ypaA mRNA.

The lysine riboswitch (also known as the L-box) binds lysine to regulate lysine biosynthesis, catabolism and transport. The Lysine riboswitch is a metabolite binding RNA element found within certain messenger RNAs that serve as a precision sensor for the amino acid lysine. Allosteric rearrangement of mRNA structure is mediated by ligand binding, and this results in modulation of gene expression. This riboswitches is found in a number of genes involved in lysine metabolism, including lysC. The lysine riboswitch has also been identified independently.

Riboswitches as antibiotic targets. Riboswitches are expected to be targets for novel antibiotics. Indeed, some antibiotics whose mechanism of action was unknown for decades have been shown to operate by targeting riboswitches. For example, when the antibiotic pyrithiamine enters the cell, it is metabolized into pyrithiamine pyrophosphate. Pyrithiamine pyrophosphate has been shown to bind and activate the TPP riboswitch, causing the cell to cease the synthesis and import of TPP. Because pyrithiamine pyrophosphate does not substitute for TPP as a coenzyme, the cell dies.

One potential advantage that riboswitches have as an antibiotic target is that many of the riboswitches have multiple instances per genome, where each instance controls an operon containing many genes, many of which are essential. Therefore, in order for bacteria to evolve resistance to the antibiotic by mutations in the riboswitch, all riboswitches must be mutated.

Disclosed are the crystalline atomic structures of riboswitches. These structures are useful in modeling and assessing the interaction of a riboswitch with a binding ligand. They are also useful in methods of identifying compounds that interact with the riboswitch.

Also disclosed are compositions and methods for selecting and identifying compounds that can activate, deactivate, or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch. Also disclosed are methods of identifying a compound that interacts with a riboswitch comprising modeling the atomic structure of the riboswitch with a test compound and determining if the test compound interacts with the riboswitch. This can be done by determining the atomic contacts of the riboswitch and test compound. Furthermore, analogs of a compound known to interact with a riboswitch can be generated by analyzing the atomic contacts, then optimizing the atomic structure of the analog to maximize interaction. These methods can be used with a high throughput screen.

Also disclosed are methods for activating, deactivating, or blocking a riboswitch. Also disclosed are compositions for activating, deactivating or blocking a riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch. The nature of the inaction between a riboswitch and a ligand other than a natural ligand can be as an agonist or an antagonist.

Also disclosed are methods for identifying compounds which are capable of altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule.

Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are compositions and methods for regulating expression of an RNA molecule, or of a gene encoding an RNA molecule, by operably linking a riboswitch to the RNA molecule. A riboswitch can be operably linked to an RNA molecule in any suitable manner, including, for example, by physically joining the riboswitch to the RNA molecule or by engineering nucleic acid encoding the RNA molecule to include and encode the riboswitch such that the RNA produced from the engineered nucleic acid has the riboswitch operably linked to the RNA molecule. Subjecting a riboswitch operably linked to an RNA molecule of interest to conditions that activate, deactivate or block the riboswitch can be used to alter expression of the RNA.

Also disclosed are compositions and methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects.

Also disclosed are compositions and methods for regulating expression of an isolated, engineered or recombinant gene or RNA that contains a riboswitch by activating, deactivating or blocking the riboswitch. The gene or RNA can be engineered or can be recombinant in any manner. For example, the riboswitch and coding region of the RNA can be heterologous, the riboswitch can be recombinant or chimeric, or both. If the gene encodes a desired expression product, activating or deactivating the riboswitch can be used to induce expression of the gene and thus result in production of the expression product. If the gene encodes an inducer or repressor of gene expression or of another cellular process, activation, deactivation or blocking of the riboswitch can result in induction, repression, or de-repression of other, regulated genes or cellular processes. Many such secondary regulatory effects are known and can be adapted for use with riboswitches. An advantage of riboswitches as the primary control for such regulation is that riboswitch trigger molecules can be small, non-antigenic molecules.

Also disclosed are compositions and methods for altering the regulation of a riboswitch by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are compositions and methods for inactivating a riboswitch by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Also contemplated are the development of isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches.

The biosynthesis of several protein cofactors is subject to feedback regulation by riboswitches. Flavin mononucleotide (FMN)-specific riboswitches, also known as RFN elements, direct expression of bacterial genes involved in the biosynthesis and transport of riboflavin (vitamin B2) and related compounds. Applicants have determined for the first time the crystal structures of the Fusobacterium nucleatum riboswitch, as bound to FMN, riboflavin, and antibiotic roseoflavin. Applicants' structural data, complemented by binding and footprinting experiments, imply a largely pre-folded tertiary RNA architecture and FMN recognition mediated by conformational transitions within the junctional binding pocket. The inherent plasticity of the FMN-binding pocket and the availability of large openings make the riboswitch an attractive target for structure-based design of FMN-like antimicrobial compounds.

The determination of the FMN riboswitch structure is especially interesting and timely, given the predicted complexity of its riboswitch fold, the abundance of this riboswitch in pathogenic species, and its involvement in riboflavin overproduction in biotechnologically relevant bacterial strains.

The inventive subject matter thus relates to a computer model generated from a data array, computer readable storage media encoded with the model, and computers comprising the model, wherein the model is derived from atomic structure coordinates of an FMN riboswitch or a lysine riboswitch. The inventive subject matter further relates to a pharmacophore having a spatial arrangement of atoms defined by the binding pocket identified in the model, along with a compound defined by the pharmacophore. Finally, the inventive subject matter additionally relates to methods for rational drug design based on the atomic structure data, and to compounds identified by the rational drug design process.

In particular, the inventive subject matter relates to a computer model of an FMN riboswitch generated from a data array comprising the atomic structure coordinates of an FMN riboswitch as set forth in any one of Tables 6-14, or a composite thereof. The data found in Tables 6-14 is X-ray diffraction data depicting the spatial coordinates of the atoms of an exemplary, consensus FMN riboswitch in crystal form and interacting with several different FMN riboswitch ligands in the presence of several possible metal cations are facilitate the interactions between the riboswitch and said ligands.

In one aspect of the inventive subject matter, said model is encoded onto a computer-readable storage medium.

In another aspect of the inventive subject matter, said model is stored in the memory of a computer. With the model and supporting data stored in memory, said computer becomes a special-purpose computer which is capable of performing molecular modeling functions, of which a general-purpose is incapable. In a preferred embodiment, the computer described immediately above additionally comprises executable code for:

(a) displaying the data array as a 3-dimensional model;

(b) analyzing the binding site of the model of an FMN riboswitch;

(c) screening in silico a library for small molecules that fit into said binding site; and (d) controlling a unit for assaying the small molecules determined in step (c) in a an FMN riboswitch binding assay.

In a further aspect of the inventive subject matter, the computer model described above is based upon a data array comprising atomic structure coordinates of an FMN riboswitch obtained in the presence of a sufficient amount of magnesium to saturate all sites for binding interactions between said FMN riboswitch and said magnesium. As discussed in the Examples below, the absence of magnesium or the substitution of another metal cation appears to substantially impair the binding interaction between an FMN riboswitch and its natural and artificial ligands.

The inventive subject matter further relates to a pharmacophore having a spatial arrangement of atoms defined by the binding pocket identified in the computer model described above.

In an alternate aspect, said spatial arrangement of atoms is determined in the presence of a sufficient amount of magnesium to saturate all sites for binding interactions between said FMN riboswitch and said magnesium.

The inventive subject matter also relates to an isolated compound, or a salt or solvate thereof, defined by the pharmacophore described above.

The inventive subject matter additionally relates to a method for identifying a compound that interacts with an FMN riboswitch, utilizing a 3-D molecular model of an FMN riboswitch as shown in any one of Tables 6-14, or a composite thereof, comprising:

(a) using said model in a method of rational drug design to identify candidate compounds that can bind an FMN riboswitch; and

(b) assaying the binding of a candidate compound identified in step (a) using a purified FMN riboswitch to thereby determine a binding characteristic, or lack thereof, of said compound.

In one aspect of the inventive subject matter, determining the binding characteristics of said compound in interaction with the riboswitch comprises determining a predicted minimum interaction energy, a predicted binding constant, a predicted dissociation constant, or a combination thereof, for the test compound in the model of the riboswitch.

In another aspect, determining if the test compound interacts with the riboswitch comprises determining one or more predicted bonds, one or more predicted other interactions, or a combination thereof, for the test compound in the model of the riboswitch.

As above, said 3-D molecular model of an FMN riboswitch is preferably determined from data obtained in the presence of a sufficient amount of magnesium to saturate all sites for binding interactions between said FMN riboswitch and said magnesium.

Further, the inventive subject matter relates to a method of killing bacteria, comprising contacting the bacteria with a compound identified by the method described above.

In yet another aspect of the inventive subject matter, a method for identifying a compound that interacts with an FMN riboswitch, utilizing the crystal structure of an FMN riboswitch, comprises the steps of:

(a) modeling the FMN riboswitch with a test compound; and

(b) determining if the test compound interacts with the FMN riboswitch.

In addition, the inventive subject matter relates to a computer model of a lysine riboswitch generated from a data array comprising the atomic structure coordinates of a lysine riboswitch as set forth in any one of Tables 15-26, or a composite thereof. The data found in Tables 15-26 is X-ray diffraction data depicting the spatial coordinates of the atoms of an exemplary, consensus lysine riboswitch in crystal form and interacting with several different lysine riboswitch ligands in the presence of several possible metal cations are facilitate the interactions between the riboswitch and said ligands.

In one aspect of the inventive subject matter, said model is encoded onto a computer-readable storage medium.

In another aspect of the inventive subject matter, said model is stored in the memory of a computer. With the model and supporting data stored in memory, said computer becomes a special-purpose computer which is capable of performing molecular modeling functions, of which a general-purpose is incapable. In a preferred embodiment, the computer described immediately above additionally comprises executable code for:

(a) displaying the data array as a 3-dimensional model;

(b) analyzing the binding site of the model of a lysine riboswitch;

(c) screening in silico a library for small molecules that fit into said binding site; and

(d) controlling a unit for assaying the small molecules determined in step (c) in a lysine riboswitch binding assay.

In a further aspect of the inventive subject matter, the computer model described above is based upon a data array comprising atomic structure coordinates of a lysine riboswitch obtained in the presence of a sufficient amount of magnesium to saturate all sites for binding interactions between said lysine riboswitch and said magnesium. As discussed in the Examples below, the absence of magnesium or the substitution of another metal cation appears to substantially impair the binding interaction between a lysine riboswitch and its natural and artificial ligands.

The inventive subject matter further relates to a pharmacophore having a spatial arrangement of atoms defined by the binding pocket identified in the computer model described above.

In an alternate aspect, said spatial arrangement of atoms is determined in the presence of a sufficient amount of magnesium to saturate all sites for binding interactions between said lysine riboswitch and said magnesium.

The inventive subject matter also relates to an isolated compound, or a salt or solvate thereof, defined by the pharmacophore described above.

The inventive subject matter additionally relates to a method for identifying a compound that interacts with a lysine riboswitch, utilizing a 3-D molecular model of a lysine riboswitch as shown in any one of Tables 15-26, or a composite thereof, comprising:

(a) using said model in a method of rational drug design to identify candidate compounds that can bind a lysine riboswitch; and

(b) assaying the binding of a candidate compound identified in step (a) using a purified lysine riboswitch to thereby determine a binding characteristic, or lack thereof, of said compound.

In one aspect of the inventive subject matter, determining the binding characteristics of said compound in interaction with the riboswitch comprises determining a predicted minimum interaction energy, a predicted binding constant, a predicted dissociation constant, or a combination thereof, for the test compound in the model of the riboswitch.

In another aspect, determining if the test compound interacts with the riboswitch comprises determining one or more predicted bonds, one or more predicted other interactions, or a combination thereof, for the test compound in the model of the riboswitch.

As above, said 3-D molecular model of a lysine riboswitch is preferably determined from data obtained in the presence of a sufficient amount of magnesium to saturate all sites for binding interactions between said lysine riboswitch and said magnesium.

Further, the inventive subject matter relates to a method of killing bacteria, comprising contacting the bacteria with a compound identified by the method described above.

In yet another aspect of the inventive subject matter, a method for identifying a compound that interacts with a lysine riboswitch, utilizing the crystal structure of a lysine riboswitch, comprises the steps of:

(a) modeling the lysine riboswitch with a test compound; and

(b) determining if the test compound interacts with the lysine riboswitch.

### Experimental Discussion

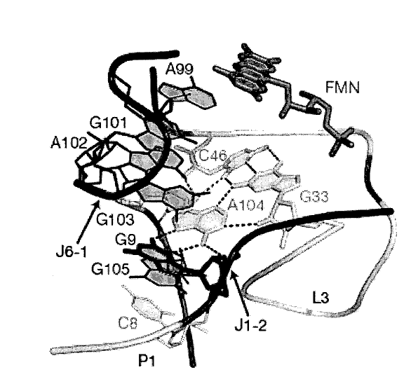

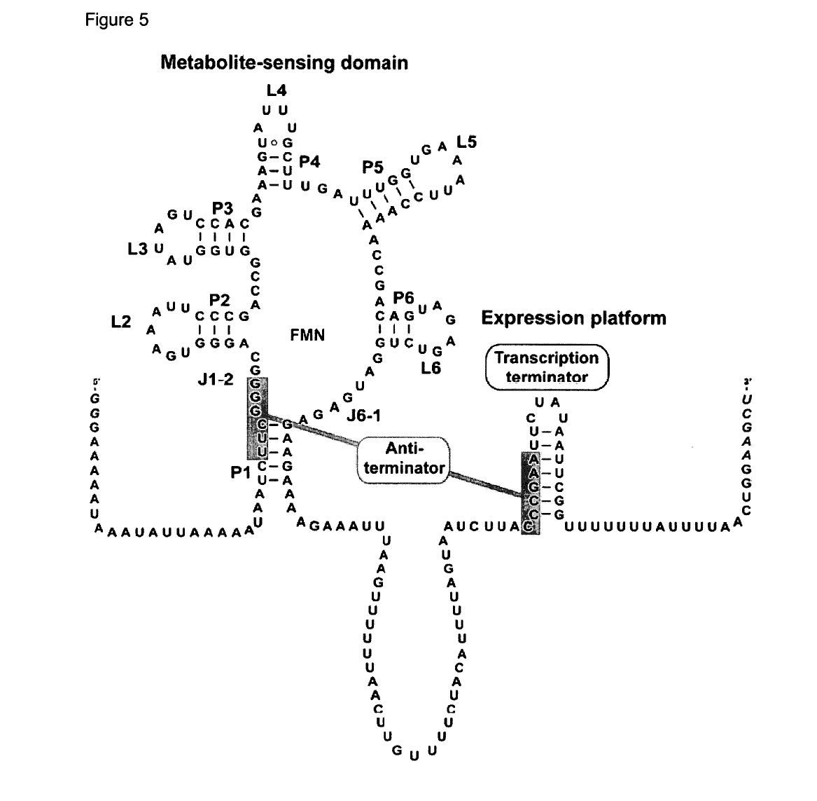

FMN riboswitches. Fusobacterium nucleatum plays a role in periodontal disease and other human infections and is considered one of the most pathogenic bacteria of the genus. The intracellular concentration of FMN in F. nucleatum is apparently controlled by a transcription attenuation mechanism involving two riboswitches, positioned before the riboflavin synthetic genes of the ribHDE(B/A) operon and the candidate riboflavin transporter impX gene (FIG. 5). In FIG. 5: Predicted transcription attenuation mechanism for the R nucleatum impX FMN riboswitch. The anti-termination mechanism has been experimentally shown for the B. subtilis FMN riboswitch. The metabolite-sensing domain of the riboswitch folds in the presence of FMN thereby facilitating formation of the PI helix. Stabilization of the FMN-bound conformation of the sensing domain promotes formation of a transcription terminator in the downstream expression platform. Transcription of the gene, therefore, is prematurely terminated. In the absence of FMN, the PI helix is not formed and regions highlighted in green participate in the formation of an alternative anti-terminator hairpin conformation. In this case, transcription of the gene is not blocked. Nucleotides not present in the gene are shown in italics. FMN binds this 220-nucleotide RNA with the Kd 134.0±13.9 nM (mean±s.d.).

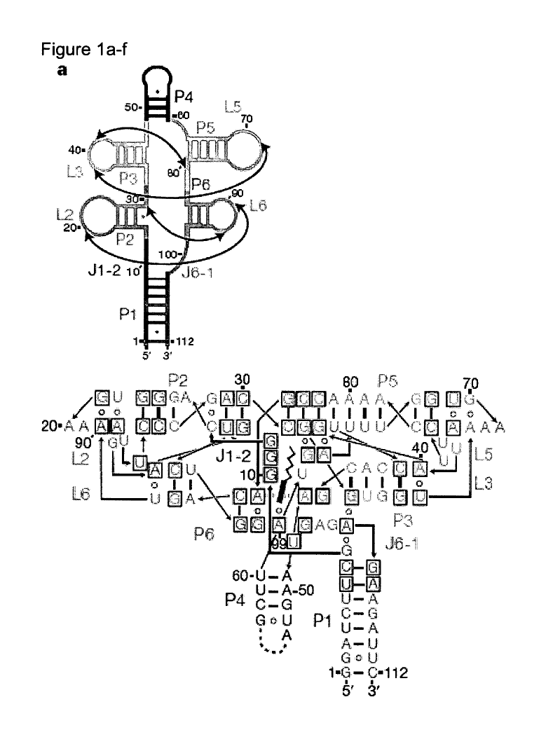

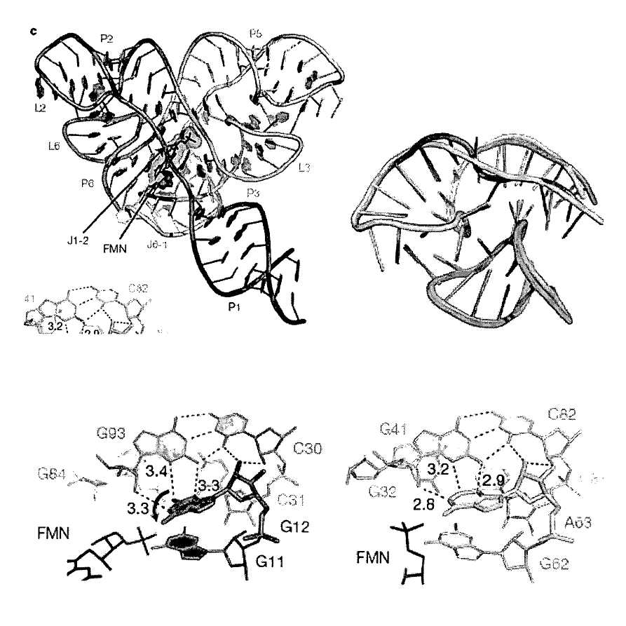

In FIG. 1: a, Homology-based schematic of the FMN riboswitch with key long-range interactions indicated by arrows. RNA segments are depicted in colors used for subsequent figures. b, Schematic of the riboswitch fold observed in the crystal structure of the complex. The bound FMN is in red. Key stacking interactions involving FMN are shown as blue dashed lines. Nucleotides that are more than 95% conserved among 183 FMN riboswitches are boxed. c, Overall riboswitch structure in a ribbon representation. d, Superposition of the P2-P6 (nucleotides 10-32 and 85-98) and P3-P5 domains (nucleotides 62-84 and 33-46). The root mean square deviation is 1.8 A°. e, f, Distinct alignments of nucleotide triples in the P2-P6 (e) and P3-P5 (f) domains. Dashed lines depict putative hydrogen bonds. Distances are in angstroms.

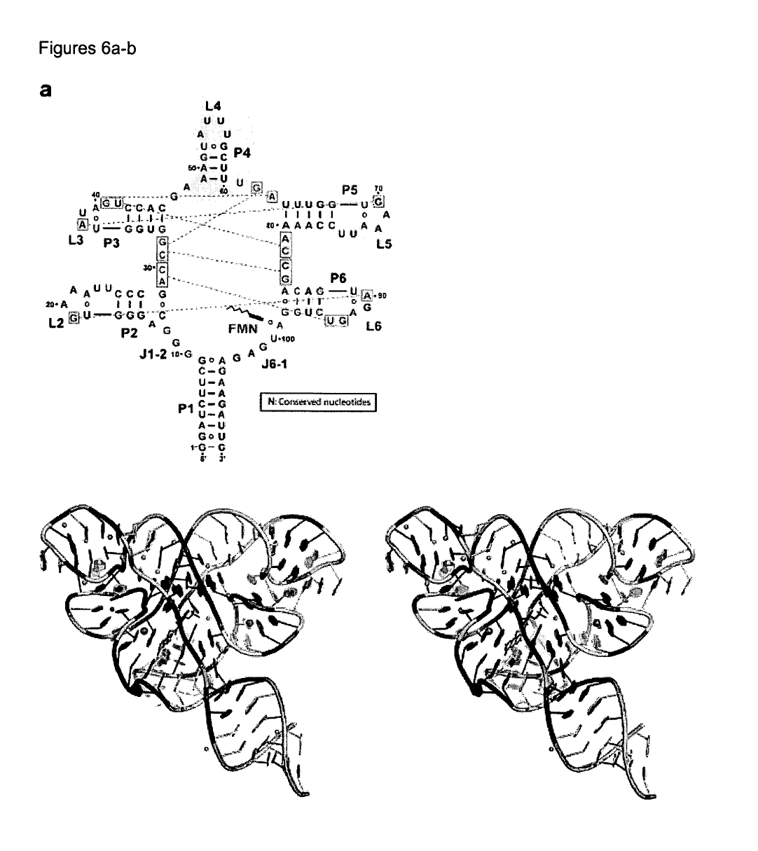

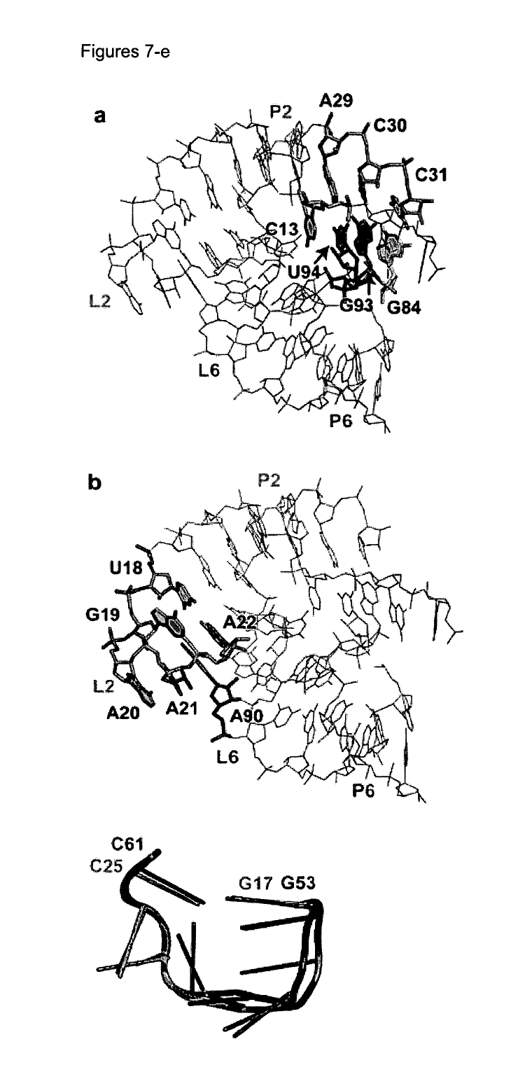

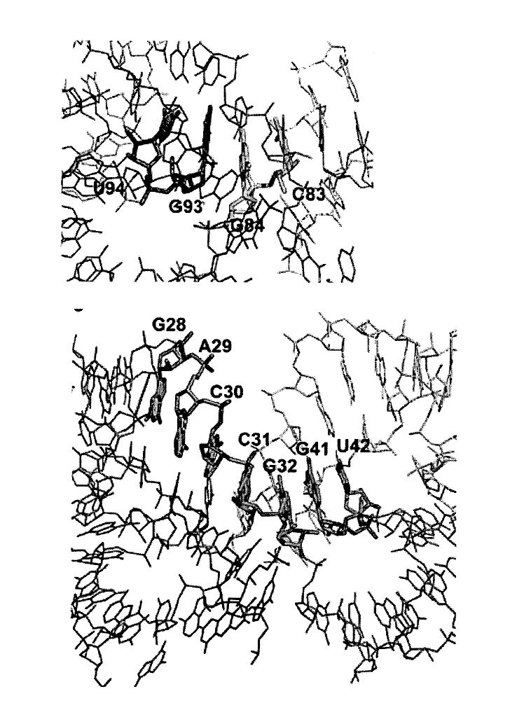

Applicants have determined the 2.95-A° structure of the FMN-bound 112-nucleotide F. nucleatum impX RFN element (SEQ ID NO: 1), which conforms well with the consensus sequence and the six-helical junctional secondary structure characteristic of this riboswitch family (FIGS. 1a-c and FIG. 6a, b). The structure features a complex FMN-bound junctional region “stapled” together by two peripheral domains, P2-P6 and P3-P5. Each peripheral domain is formed by two interacting stem-loops, stabilized by two pairs of tertiary contacts involving loop-loop (L2-L6 and L3-L5) and loop-helix (L6-P2 and L3-P5) interactions (FIG. 1a and FIG. 7a, b), resulting in an overall butterfly-like fold (FIG. 1c). Stems P1 and P4 radiate in opposite directions from the bottom part of the junction. Most unexpectedly, the peripheral domains show nearly identical conformations when superposed by 180° rotation along the axis directed through the central region of the riboswitch (FIG. 1d).

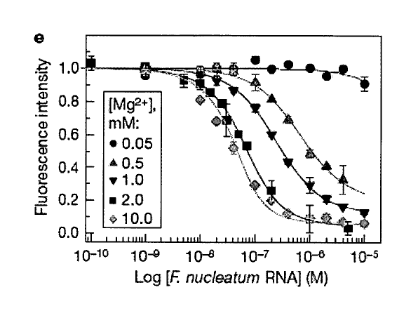

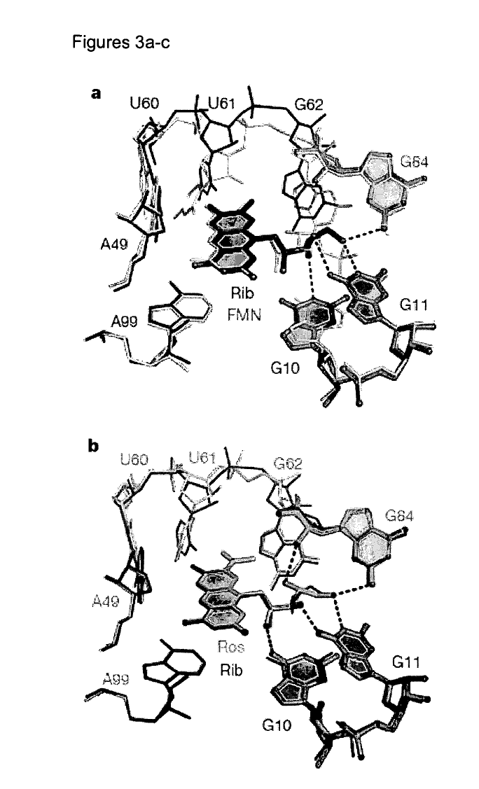

In FIG. 3: a, All-atom superposition of the ligand-binding pocket for riboflavin-bound (blue and green) and FMN-bound (grey) riboswitches. Nucleotides in green are positioned within hydrogen-bond distances of the ribityl moiety of riboflavin. b, Superposition of riboflavin-bound (blue) and roseoflavin bound (pink and green) riboswitches, depicted as in a. c, Surface view inside of the FMN-bound riboswitch with large openings shown with red arrows.

In FIG. 6. Architecture and sequence conservation of the FMN riboswitch. a, Secondary structure schematic of the F nucleatum FMN riboswitch used in the study. The schematic is based on the models from refs. 5, 8, 17. Stem-loops are color-coded similarly to those in FIG. 1. Nucleotides conserved in >95% riboswitches among 183 sequences from Rfam data base 36 are in red color. Nucleotides participating in tertiary contacts are squared and connected by dashed lines. b, Three-dimensional structure of the F nucleatum FMN riboswitch (ribbon representation) in stereo view. Stem-loops and conserved nucleotides are color-coded as in panel (a). Violet and green spheres represent K+ and Mg²⁺ cations.

In FIG. 7: Examples of tertiary structure elements of the F. nucleatum FMN riboswitch. a, Interactions between loop L6 and helix P2, stabilized by formation of A29-U94 and C30-G93 base pairs, and by stacking interactions between C13, U94, G93 and G84. b, Intercalation of A90 of loop L6 into T-loop motif of loop L2. c, All-atom superposition of T-loop motifs from loop L2 (nts 17-25) and tRNA(PDB ID lEHZ) (nts 53-61). The root mean square deviation (r.m.s.d.) is 2.1 angstrom. d and e, Continuous stacking interactions that provide connection between helices P6 and P5 (d), and P2 and P3 (e).

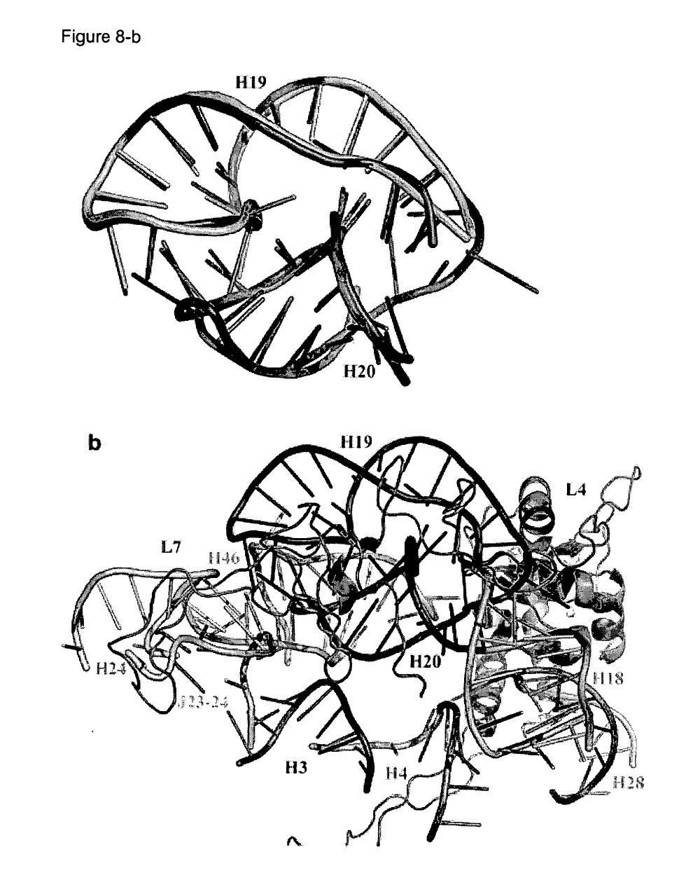

The pseudo-symmetrical FMN does not take advantage of the two-fold symmetry between riboswitch elements, because its isoalloxazine ring and phosphate are oriented towards different riboswitch domains. The peripheral domains contain small RNA motifs, such as a T-loop (FIG. 7c), and show a striking resemblance to larger architectural modules found in 23S ribosomal RNA (rRNA) (FIG. 8). Despite this similarity, the domains serve as molecular staples to hold and shape the FMN-binding pocket in the riboswitch, whereas in rRNA they form stable platforms for interactions with proteins and other rRNA segments.

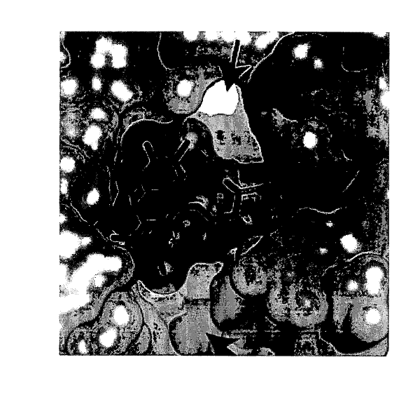

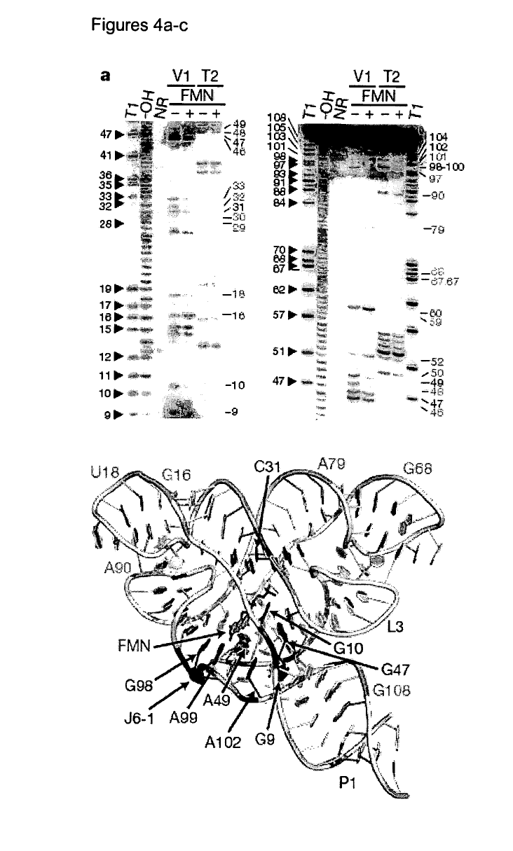

In FIG. 4. Probing the FMN riboswitch tertiary structure. a, Probing of 59 32P-labelled 112-nucleotide RNA (0.5 mM) by V1 and T2 nucleases in the absence and presence of sixfold excess of FMN. T1 and —OH designate RNase T1 and alkaline ladders, respectively. NR, no reaction. Weak and strong FMN-induced cleavage protections are shown in light and dark red, whereas weak and strong cleavage enhancements are shown in light and dark green. b, Projections of the nuclease cleavage reductions (light and dark red) and enhancement (green) on the riboswitch structure. c, Nucleotides potentially facilitating formation of the regulatory P1 helix. Hydrogen bonds in the G9NA104N(G33-C46) tetrad are indicted. FMN-induced cleavage reductions in the in-line probing assay 5 are in red.

In FIG. 8. Comparison of the P3-P5 domain of the FMN riboswitch and the H19-H20 domain (nucleotides 299-339) of the Thermus thermophilus 23S rRNA23 (PDB ID 2J03). a, All-atom superposition of the FMN riboswitch domain (yellow and cyan; nucleotides 33-37, 38-46, 62-73, and 75-84) and 23S rRNA (magenta; nucleotides 324-328, 330-338, 299-310, 311-320). R.m.s.d. is 1.48 A for the superposed residues. b, H19-H20 domain (magenta) in the context of ribosome. The RNA segments (various colors) and proteins (green) surrounding the domain are shown. The H19-H20 domain also shows similarity to the H25.1-H72 domain of the 23S rRNA (1FFX) from Haloarcula marismorti 37.

Comparison of the loop-helix interactions uncovers a small difference that may contribute to gene expression regulation. In the P2-P6 domain, invariant G12 from the G12N(G93-C30) triple (FIG. 1e) replaces the corresponding A63 in the stable A-minor A63N(G41-C82) triple from the P3-P5 domain (FIG. 1f). To prevent a steric clash with the G93-C30 pair, the G12 base is moved out of the triple plane, thereby weakening both G12N(G93-C30) and G11N(G84-C31) triples. The lower stability of these adjacent triples could enhance the mobility of the J1-2 segment, highlighting its contribution to the coenzyme-sensitive switch governing gene regulation. In essence, the J1-2 segment participates in anti-terminator formation in the absence of FMN, whereas in the FMN-bound state it is locked up in the junction, thereby facilitating formation of the regulatory P1 helix.

In contrast to other multi-stem junctional riboswitches, the junctional region of the FMN riboswitch is not constructed on the basis of collinear stacking of adjacent helices, but instead is composed of several non-paired segments, which provide a smooth transition between adjacent helices (FIG. 1c and FIG. 7d,e). Unlike coenzymes in other riboswitches, the oxidized FMN is positioned centrally inside the junctional region and is surrounded by all six RNA stems (FIGS. 1c and 2a). The planar isoalloxazine ring system intercalates between A48 and A85, thereby providing a continuous stacking alignment linking P6 and P3 helices. The uracil-like edge of the ring system forms specific Watson-Crick-like hydrogen bonds with the highly conserved A99 (FIG. 2b). The ribityl moiety of FMN uses only one of its four oxygens for hydrogen bonding, whereas phosphate oxygens form additional hydrogen bonds with Watson-Crick edges of several conserved guanines. The interaction between the phosphate of FMN and RNA is also bridged by metal ion Ml, assigned as Mg⁺², which directly coordinates the phosphate oxygen of FMN and the N7 position of G33, and forms several water-mediated contacts with neighboring nucleotides (FIG. 2c).

In FIG. 2: a, A view of the junction with bound FMN. Magenta spheres depict cations in proximity to FMN. b, Details of riboswitch-FMN interactions. Hydrogen bond distances are shown with dashed lines. c, FMN-riboswitch interactions mediated by M1, assigned to an Mg⁺² cation. Magenta sticks depict coordination bonds. Mg⁺²-coordinated water molecules (blue spheres) are positioned on the basis of direct coordination bonds with phosphate and G33. d, Chemical structures (left panel) and fluorescent binding assay (right panel) of FMN and its analogues with the 112-nucleotide F. nucleatum riboswitch conducted in 2 mM MgCl₂ and 100 mM KCl. The dissociation constants (micromolar, mean6s.d.) are: FMN, 0.037560.0031; riboflavin, 39.8; lumiflavin, 59.0. e, Interaction of FMN with F. nucleatum riboswitch in 100 mM KCl as a function of variable Mg⁺² concentration. The dissociation constants (nanomolar, mean6s.d.) are: 0.5 mM MgCl₂, 406.0652.7; 1.0 mM MgCl₂, 211.762.1; 10.0 mM MgCl₂, 10.962.4.

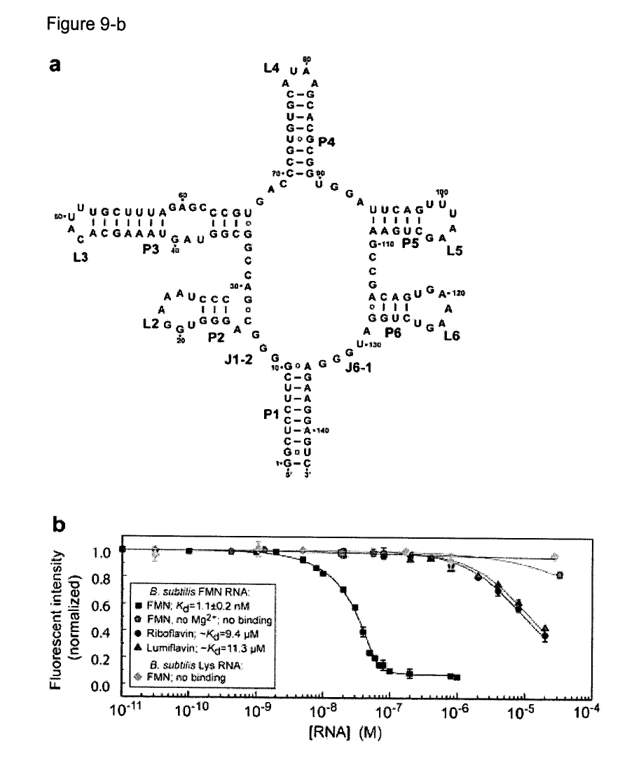

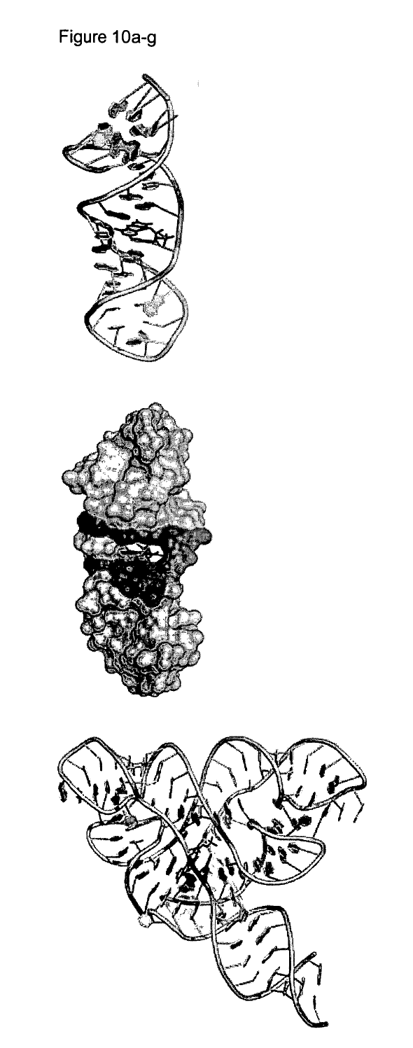

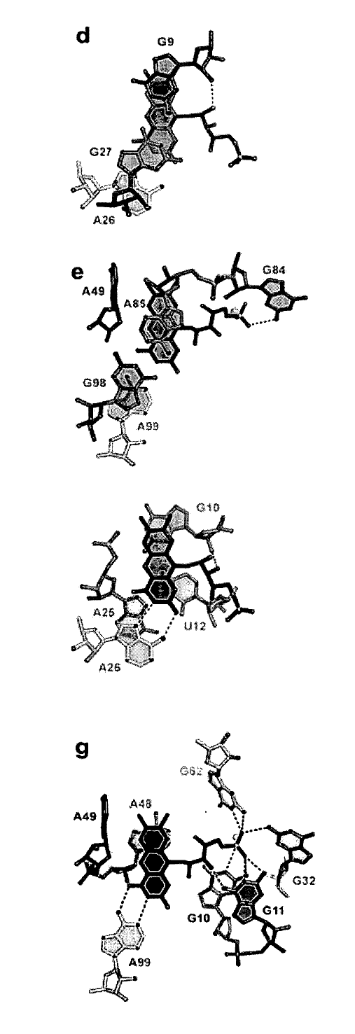

In support of the structural data, the binding affinity of riboflavin, the FMN precursor which lacks the phosphate and does not control gene expression, decreases by about 1,000-fold, compared with binding of FMN to both F. nucleatum and Bacillus subtilis riboswitches (FIG. 2d and FIG. 9). The additional removal of the ribityl moiety in lumiflavin reduces binding affinity to only a very small extent (FIG. 2d). In contrast to the FMN riboswitch, the in-vitro-selected RNA aptamer does not bind the phosphate moiety of FMN and uses the Hoogsteen edge of adenine for specific recognition of the ring system (FIG. 10).

In FIG. 9. The metabolite-sensing domain of the B. subtilis FMN riboswitch. a, The sequence of the 143-nucleotide fragment of B. subtilis riboswitch 5 is presented as a secondary structure schematic, which is based on the consensus secondary structure of the FMN riboswitches with small corrections from the three-dimensional structure of the F nucleatum riboswitch. b, Binding of FMN and its analogs to the 143-nucleotide B. subtilis FMN riboswitch. All measurements were performed using fluorescent assay. RNA was titrated against 6×10-8 M of FMN and 10-8 M of riboflavin or lumiflavin. Each experiment was performed-2-4 times in 50 mM Tris-HCI, pH 7.4, 100 mM KCl, 2 mM MgCl₂. The 172 nucleotide fragment of the B. subtilis lysine riboswitch 27 was used as a negative control.

In FIG. 10. Comparison of the structures of the in-vitro-selected FMN-specific aptamer RNA and the FMN riboswitch. a, b, c, Ribbon (a) and surface (b) representations of the FMN-bound aptamer RNA28 and the FMN riboswitch (c). In contrast to the FMN riboswitch, FMN (red) in the complex with the aptamer intercalates into the distorted RNA helix, leaving a large opening along the short hydrophobic and long edges of the isoalloxazine ring. Major part of the ribityl-phosphate moiety does not bind RNA and protrudes outwards. d, e, Nucleotides located above (green) and in the same layer (grey and blue) with FMN in the complexes with the aptamer (d) and riboswitch (e) RNAs. FMN is stacked on the 09-027 base pair in the aptamer complex, and on the A8S in the riboswitch complex. Hydrogen bond distances are depicted with dashed lines. f, g, Nucleotides located below (cyan) and in the same layer (grey, blue and yellow) as FMN in the complexes with the aptamer (f) and riboswitch (g) RNAs. FMN is stacked on the (A2S-UI2)-010 triple and A48 in the aptamer and riboswitch complexes, respectively. Unlike the FMN riboswitch, the aptamer makes specific hydrogen bonds with the Hoogsteen edge of adenine.

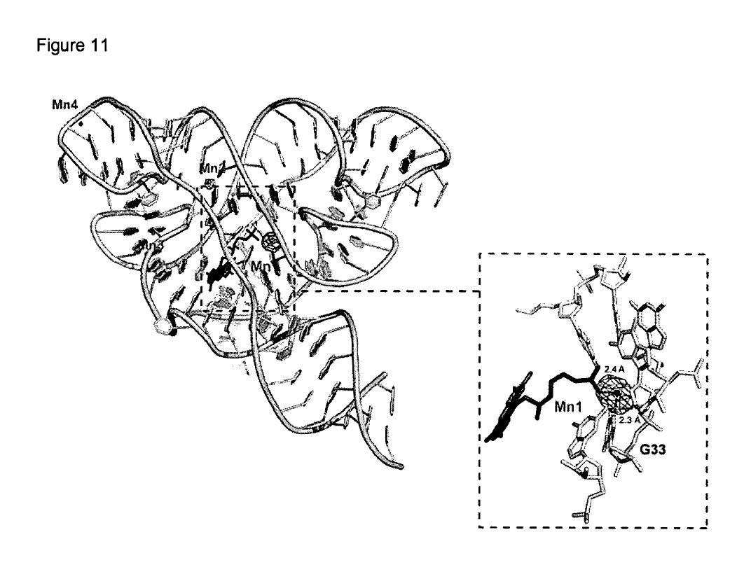

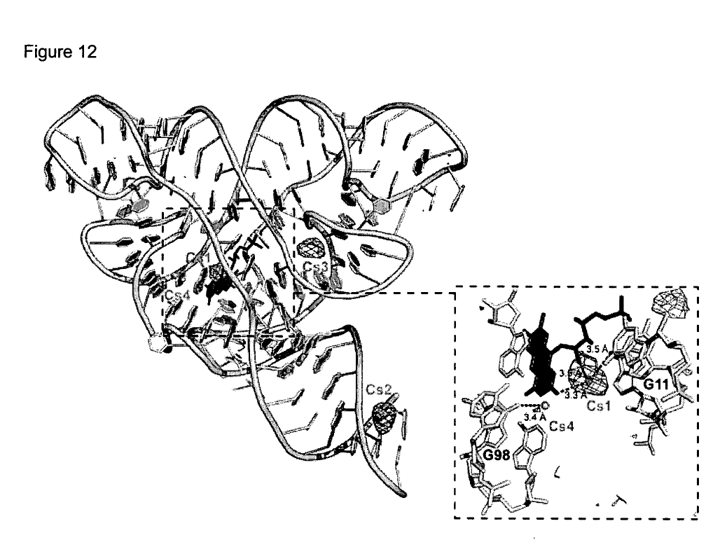

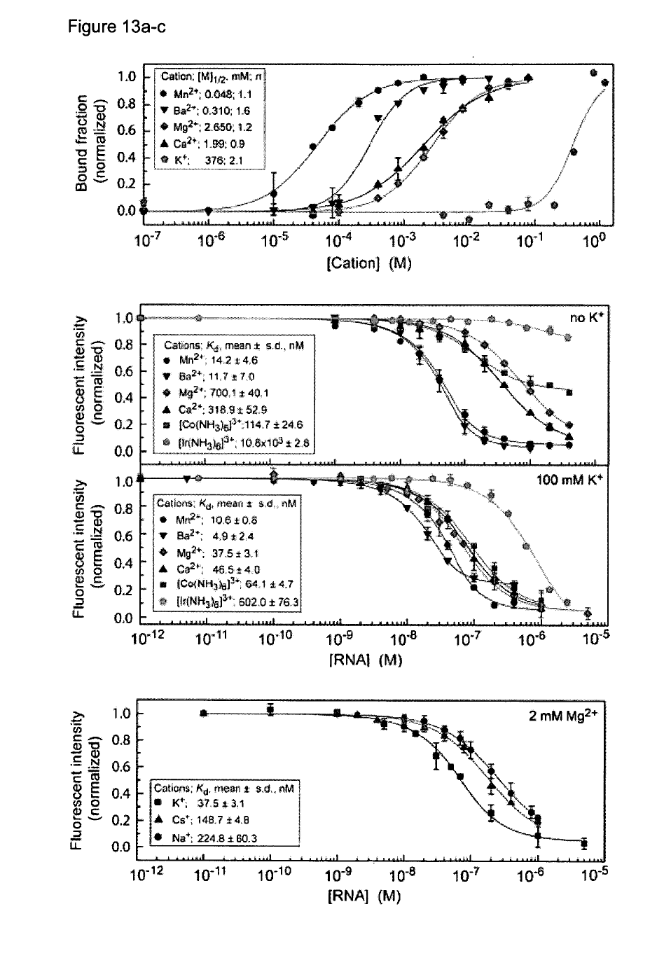

The identity of metal M1 as Mg⁺² has been confirmed by substitution with Mn⁺², a mimic of Mg⁺² (FIG. 11). Cs⁺¹ cations failed to replace M1 and were found in the P3 stem and next to the FMN ring system (FIG. 12), where a Cs⁺¹ replaces a K⁺¹ (labeled M2, FIG. 2a) cation. The interactions between FMN and the riboswitch significantly depend on the physiological concentration of Mg⁺² (FIG. 2e and FIG. 13a), and can be enhanced further by addition of 100 mM K⁺¹ (FIG. 13b). Smaller and larger monovalent cations, such as Na⁺¹ and Cs⁺¹, potentiate FMN binding to a lesser extent than K⁺¹ (FIG. 13c).

In FIG. 11: Mn2+ binding sites in the FMN riboswitch. The refined FMN riboswitch model (Mn2+ anomalous data) superposed with the anomalous map (pink) contoured at the 4.0 cr level. Mn2+ cations are shown as green spheres. Mn1 (˜11.5 cr level) is directly coordinated to the N7 position of G33 and the non-bridging phosphate oxygen of FMN and occupies the position of the FMN-bound Mg²⁺ cation M1 in the native structure of the FMN-riboswitch complex. Other Mn2+ cations also replace Mg²⁺ cations found in the native structure. Inset: the shortest distances from cations to FMN and RNA are indicated by dashed lines.

In FIG. 12: Cs+ binding sites in the FMN riboswitch. The refined FMN riboswitch model (Cs+ anomalous data) superposed with the anomalous map (pink) contoured at the 3.5 (J level. Cs+ cations are shown as green spheres. Cs1 and Cs2 occupy positions not identified as cation-binding sites in the native FMN-riboswitch structure, while Cs3 and Cs4 replace K+ cations found in the native structure. Inset: the shortest distances from cations to heteroatoms of FMN and RNA are indicated by dashed lines.

In FIG. 13: Effects of cations on the binding of FMN to the F. nucleatum FMN riboswitch. All measurements were performed using fluorescent assay and the 112-nucleotide riboswitch fragment. a, Dependence of the FMN binding upon cation concentration. Cations were titrated against RNA (2×10-7 M)-hg and (6×10-8 M) complex in 50 mM Tris-HCI, pH 7.4. Data were fitted to the equation (2) (see Methods). b, Effects of 2 mM divalent cations on the FMN-riboswitch interactions in the absence (top) and presence (bottom) of 100 mM KCl. c, Effects of 100 mM monovalent cations on the FMN riboswitch interactions in the presence of 2 mM MgCl. In both (b) and (c) RNA was titrated against 6×10-8 M of FMN in 50 mM Tris-HCI, pH 7.4, supplemented with different cations. Data were fitted to the equation (1) (see Methods).

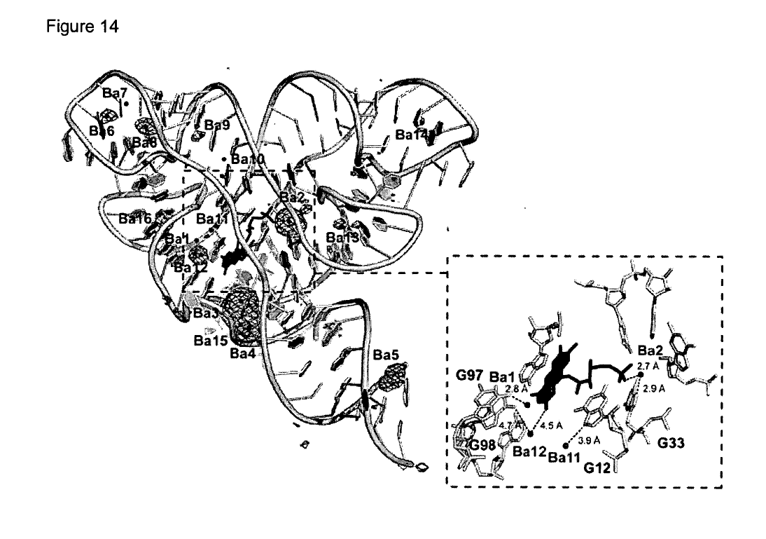

In FIG. 14: BaH binding sites in the FMN riboswitch. The refined FMN riboswitch model (Ba2+ anomalous data) superposed with the anomalous map (pink) contoured at the 3.0 cr level. Ba2+ cations are shown as purple spheres. Ba1 and Ba13 replace K+ cations; Ba7, Ba9, and Ba10 replace Mg²⁺ cations; and Ba2 replaces FMN-bound Mg²⁺ found in the native structure of the FMN-riboswitch complex. Other Ba2+ cations occupy positions not identified as cation-binding sites in the native FMN riboswitch structure. Ba2, Ba10, and Ba16 correspond to the positions of the Mn2+ cations; Ba5 and Ba13 correspond to the positions of Cs+ cations, while another Cs+ cation is positioned between Ba1 and Ba11. Ba1, Ba4, Ba5, Ba8, Ba11, Ba12, Ba14 and Ba15 are located close to [Ir(NH3)6]3+ groups. Inset: the shortest distances from Ba2+ cations to FMN and RNA are indicated by dashed lines.

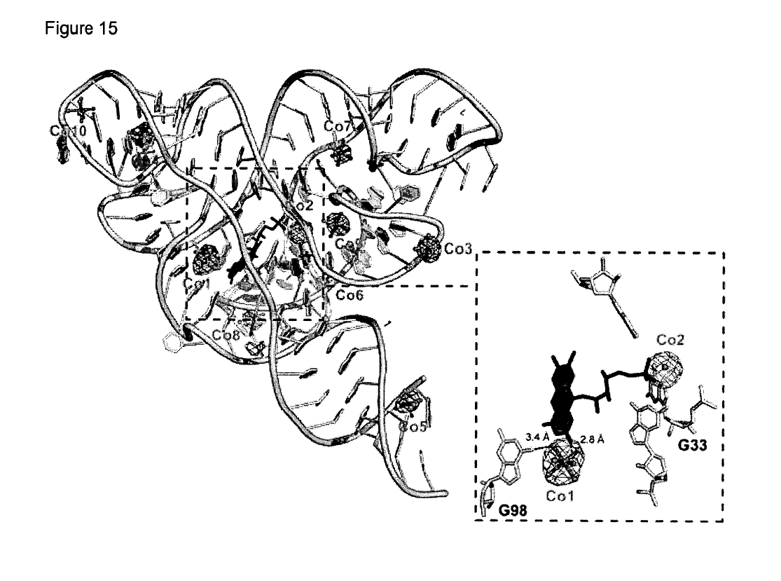

In FIG. 15: [Co(NH3)6]3+ binding sites in the FMN riboswitch. The refined FMN riboswitch model (Co anomalous data) superposed with the anomalous map (pink) contoured at the 4.0 cr level. [Co(NH3)6]3+ groups are shown as aquamarine spheres. All [Co(NH3)6]3+ groups, except Co2, are positioned similar to the [Ir(NH3)6]3+ groups. Co2 is located at the site of the FMN-bound Mg²⁺ cation M1. At this position, Co2 group forms close contacts with FMN and RNA, suggesting conformational adjustments in both RNA and phosphate moiety of FMN in order to accommodate bulky [Co(NH3)6]3+ group. These conformational changes, however, cannot be traced in the current structure due to the large proportion of the Mg²⁺-bound riboswitch form in the crystal, which may account for ˜90% of the complex (Mg²⁺ to [Co(NH3)6]3+ ratio was 8 to 1). Therefore, Co2 position has been refined as a Mg²⁺ cation. Inset: the shortest distances from cations to FMN and RNA are indicated by dashed lines.

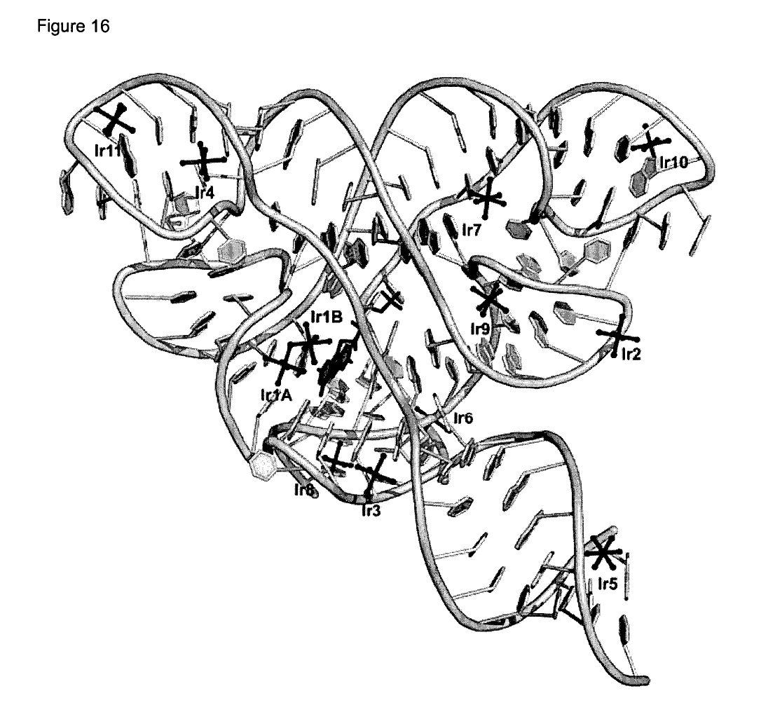

In FIG. 16: [Ir(NH3)6]3+ binding sites in the FMN riboswitch. The refined FMN riboswitch model with 11 [Ir(NH3)6]3+ groups shown in blue. 9 [Ir(NH3)6]3+ sites with high occupancy have been identified during structure phasing. 1r1 is located close to the K+ binding site M2 in the native structure of the FMN-riboswitch complex. 1r11 is occupied by Mg²⁺, and 1r7 site is occupied by a density assigned to a water molecule in the native structure.

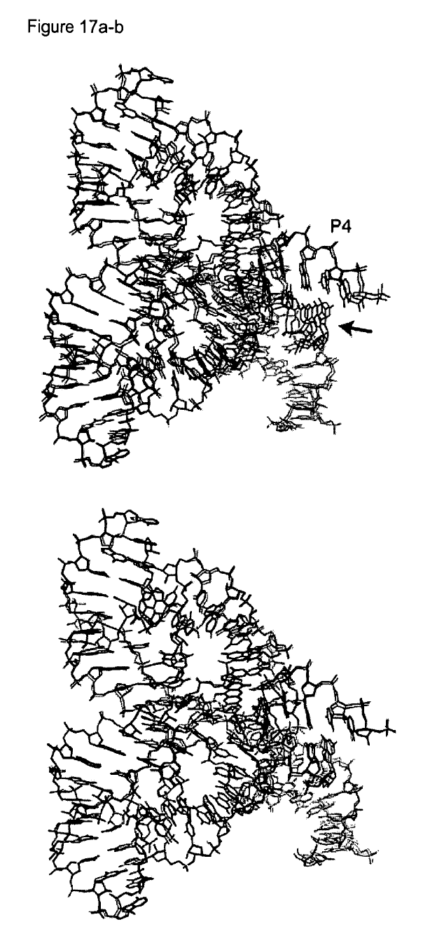

In FIG. 17: Superposition of the FMN riboswitch-ligand complexes. a, All-atom superposition of the FMN-bound (red) and riboflavin-bound (blue) riboswitches. R.m.s.d. is 0.62 angstrom. The riboflavin-bound riboswitch structure shows a small shift of P4 helix, indicated by an arrow, towards the core of the molecule. b, All-atom superposition of the roseoflavin-bound (green) and riboflavin-bound (blue) riboswitches. R.m.s.d. is 0.36 angstrom. All crystals used for comparison were grown with RNA prepared by annealing of the RNA oligonucleotides. Note that native structures of the FMN-riboswitch complexes prepared using transcribed RNA and RNA prepared from oligonucleotides are virtually identical.

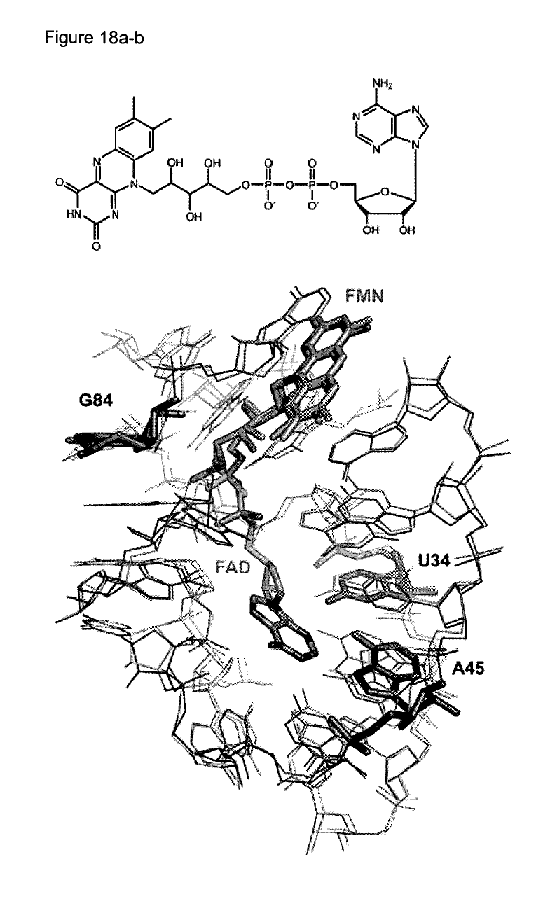

In FIG. 18: A hypothetical model of the FAD-riboswitch complex. a, Structural formula of FAD. b, A model showing the central region of the FMN riboswitch (dark green) with the bound FAD (light green, stick representation) and the corresponding region of the FMN-bound structure (grey). Nucleotides, whose conformations need to be adjusted to fit FAD into the pocket, are shown in stick representations. To build a model, FAD was inserted into the pocket on the basis of the bound FMN in the FMN-riboswitch structure using TURBO-FRODO. The model was then refined using REFMAC and experimental data, collected from the riboswitch crystals grown in the presence of FAD. The experimental data showed electron density map only for the FMN moiety of FAD, and did not show any density for the remaining part of the molecule. Therefore, the placement of the adenine moiety in the model is not based on the experimental data. It is likely that the labile phosphoanhydride bond of FAD was hydrolyzed to produce FMN during crystallization. The resulting map, when refined with FMN, did not show significant deviations from the map of the FMN-bound structure.

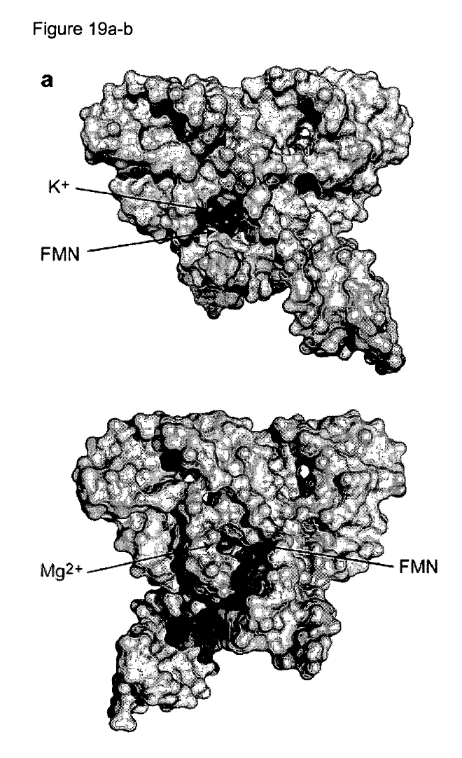

In FIG. 19: Surface views of the FMN riboswitch bound to FMN. FMN and K+ and Mg²⁺ cations, proximate to FMN, are shown in red, violet and magenta colors. a, Front view, similar to the view on Fig. b, Back view.

The identity of the divalent cation appears not to be crucial for FMN binding, because Mg⁺² can be substituted by Ca⁺², as well as and Mn⁺², even in the absence of monovalent cations (Supplementary FIGS. 9a, b and 10). It is likely that cation M1 contributes significantly to the metal dependence of the FMN binding, because the coenzyme binds the riboswitch equally well in the presence of cobalt hexamine, which can be accommodated with some adjustments in the M1 position (Supplementary FIGS. 9b and 11), whereas bulkier iridium hexamine, which does not fit into the M1 position, reduces binding affinity about 15-fold (Supplementary FIGS. 9b and 12). Unlike the M1 site, the M2 position constitutes part of a larger cation-binding area, which extends towards G11 and FMN and which can accommodate all soaked metals, except Mn⁺². Though cations in this area stabilize the FMN-binding pocket, the weaker dependence of FMN binding on monovalent cations (FIG. 13a, b) suggests a less critical role of the M2 site for FMN-riboswitch complex formation.

To gain further insights into the ligand discrimination by FMN riboswitches, Applicants have determined crystal structures of the riboswitch in complex with riboflavin and roseoflavin 7 (FIG. 3a, b). In the riboflavin-bound structure, the P4 helix is shifted towards the ligand-binding pocket (Supplementary FIG. 13a) and several nucleotides that are close to the phosphate-binding cavity and the hydrophobic edge of the ring system become slightly re-positioned (FIG. 3a). The roseoflavin-bound structure adopts a conformation similar to the riboflavin-bound structure (Supplementary FIG. 13b), with additional spatial adjustments of U61 and G62 to accommodate the dimethylamino group that substitutes for a methyl on the hydrophobic edge of the isoalloxazine ring system (FIG. 3b). Such ligand dependent conformational changes have been described earlier only for the TPP riboswitch 11. In contrast to similar recognition of the ring system, the ribityl moieties of all three FMN riboswitch ligands adopt slightly different conformations and exhibit somewhat different interactions with the RNA. As expected, no metal M1 was found in the two analogue complexes, given that analogues lack a phosphate group. The transcription of the mRNA that contains the FMN riboswitch was found to be inhibited in vitro by another coenzyme, flavin adenine dinucleotide (FAD)4, used at a 17-fold higher concentration than FMN.

Applicants' structure-based modeling of a FAD-riboswitch complex supports the possibility of FAD-mediated control of the FMN riboswitch because FAD can be accommodated within the binding pocket after minor conformational adjustments (FIG. 18). The observed plasticity of the ligand-binding pocket, together with the large openings next to the edges of the ring system (FIG. 3c) and the phosphate-binding site (FIG. 19), makes the FMN riboswitch an attractive target for the structure based design of FMN-like antimicrobial compounds.

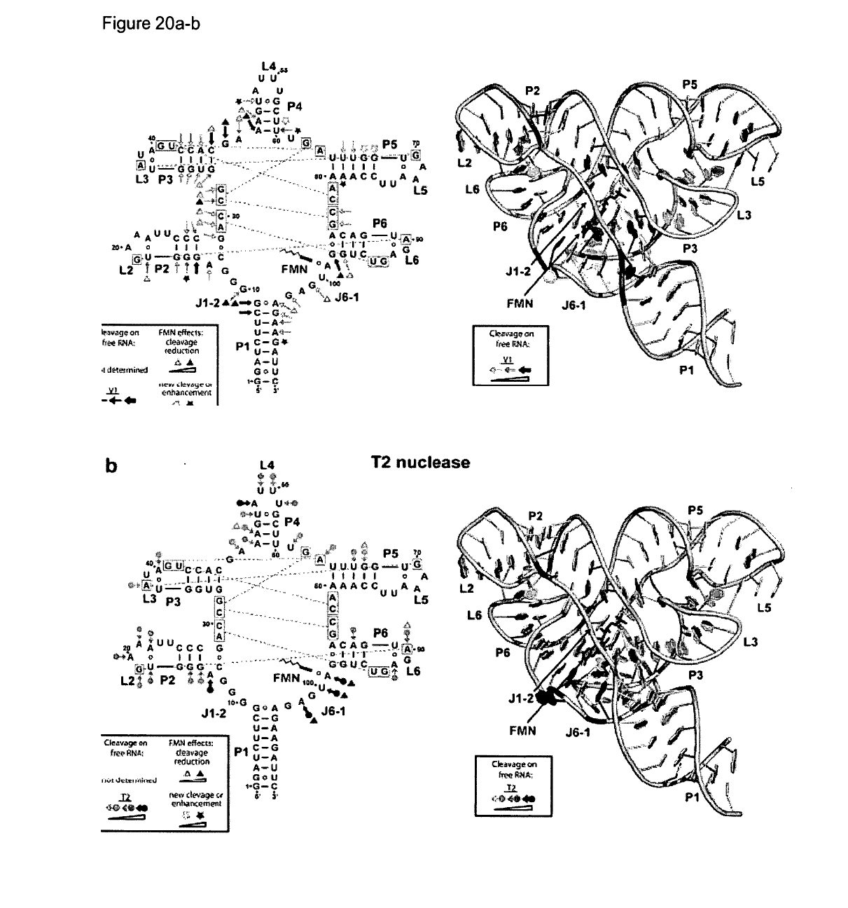

Because the bound FMN is enveloped by the RNA, the FMN riboswitch is anticipated to re-arrange its conformation on complex formation. To access these potential conformational changes, Applicants have performed footprinting experiments using nucleases V1 (paired and stacked regions) and T2 (single-stranded regions) (FIG. 4a). Despite similar conformations of the peripheral domains, the P2-P6 domain is more accessible to both nucleases (FIG. 20), and exhibits more pronounced FMN-induced changes (FIG. 4b) than its P3-P5 counterpart, implicative of a more rigid conformation for the P3-P5 domain. Nevertheless, the FMN-induced changes in the peripheral domains are not extensive and are consistent with a largely pre-formed conformation for these regions. In FIG. 20: Footprinting results for the F. nucleatum FMN riboswitch. The figure summarizes data from FIG. 4a and complements FIG. 4b. (a) Summary of VI nuclease footprinting experiments performed on the F nucleatum riboswitch projected on the secondary structure schematic (left) and the three-dimensional structure (right). Nucleotides participating in tertiary contacts are squared and connected by dashed lines. (b) Summary of T2 nuclease footprinting experiments shown as in panel (a).

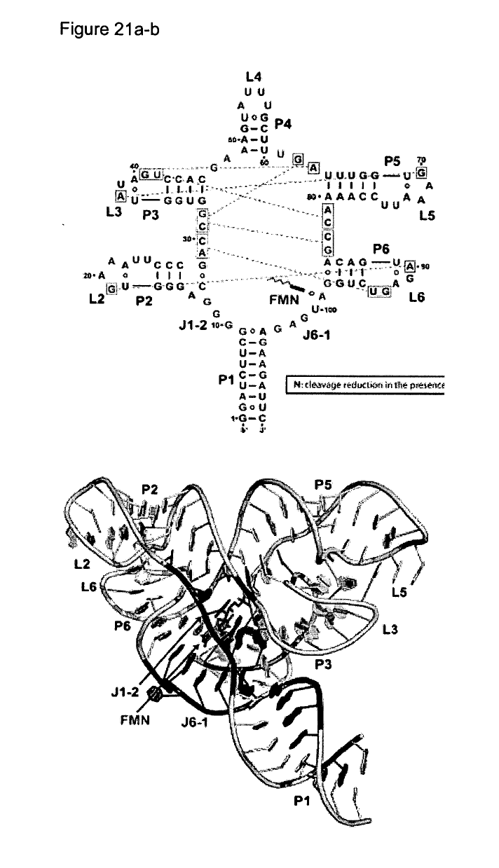

The presence of V1 and the absence of T2 cleavages indicate that nucleotides of the junctional segments are most likely involved in stacking interactions in the unbound state. Most of these nucleotides, which are clustered around the phosphate (nucleotides 10, 29-33) and isoalloxazine ring system (nucleotides 46-50, 97-102) of FMN and are adjacent to the P1 helix (nucleotides 9, 104), become protected on FMN binding owing to the mobility restrictions and shielding by neighboring RNA segments. Applicants' structural and footprinting data, corroborated by the in-line probing results on the B. subtilis riboswitch (FIG. 21), suggest that the primary recognition event(s) may involve Mg⁺²-mediated interaction of the FMN phosphate with RNA, coupled with intercalation and base-specific interactions involving the isoalloxazine ring system. In FIG. 21: In-line probing of FMN riboswitch. Projection of in-line probing data for the B. subtilis riboswitch from ref. 5 on the F nucleatum riboswitch secondary structure schematic (top) and the three-dimensional structure (bottom). These interactions should stabilize the J1-2 segment (G10-G12) and, in accordance with the strongest protections (FIG. 4a, b), bring the nuclease-accessible J6-1 segment (A99-G104) and the C8-G9 step close to each other. Such positioning provides partial stacking between the J6-1 segment (A99-G104) and the helix P1-forming strands, and facilitates formation of the non-canonical G10NG47 pair, the type I A-minor motif based G9NA104N(G33-C46) tetrad, the C8-G105 base pair, and other base pairs of the P1 helix (FIG. 4c).

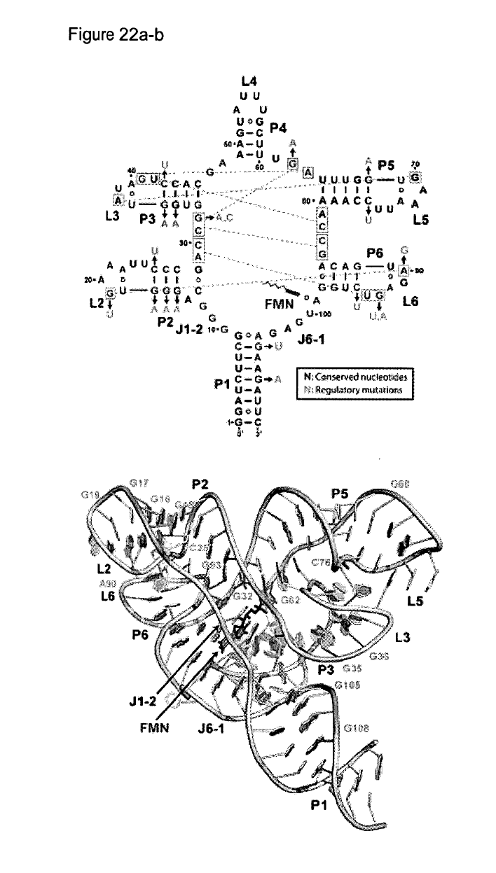

Because riboflavin biosynthetic capability is lacking in higher animals, riboflavin is traditionally used for food and feed fortification. Riboflavin can be produced in bacterial strains selected as roseoflavin resistant mutants with deregulated riboflavin biosynthesis. The FMN riboswitch structure (FIG. 22) readily explains the effects of deregulated mutations. Surprisingly, most of the mutations are concentrated in the peripheral domains, where they destabilize the helices or directly disrupt tertiary contacts. Mutations G32A/C and G62A found in the FMN-binding pocket disrupt the G62N(C83-G32) triple (FIG. 1f), which interacts with the Mg⁺²-coordinated phosphate of FMN. Two other mutations, G105U and G108A, prevent formation and/or affect stability of the regulatory P1 helix. In FIG. 22: Point mutations in the sensing domain of the FMN riboswitch causing resistance to roseoflavin and deregulation of the gene expression. Projection of known point mutations in the sensing domains of B. subtilis 4,38-40, B. amyloliquefaciens 40, Lactococcus lactis 29, Leuconostoc mesenteroides 15, and Propionibacterium freudenreichii 15 FMN riboswitches on the sequence (top) and structure (bottom) of the F nucleatum FMN riboswitch. Mutations causing the derepression of the riboswitch-controlled gene expression and resistance to roseoflavin are indicated by arrows and pink color. Nucleotides conserved in >95% riboswitches presented in Rfam36 data base are in red color.

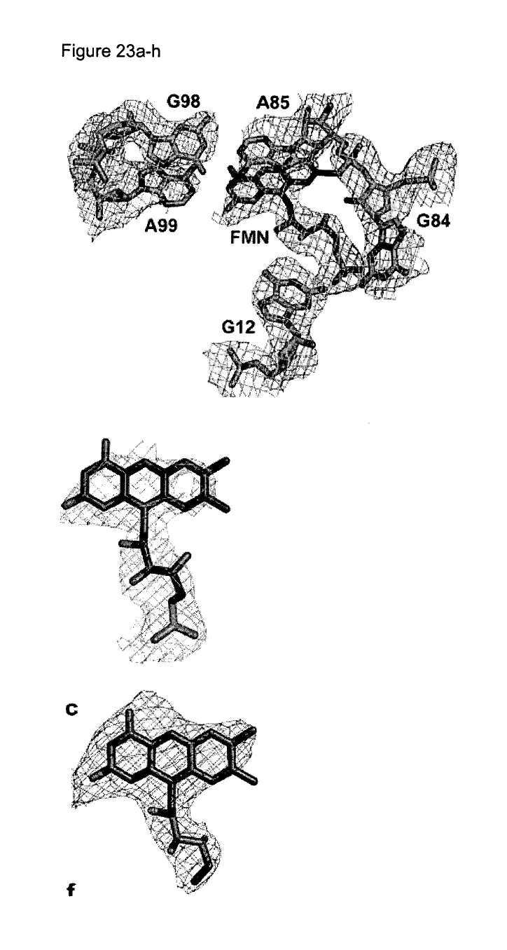

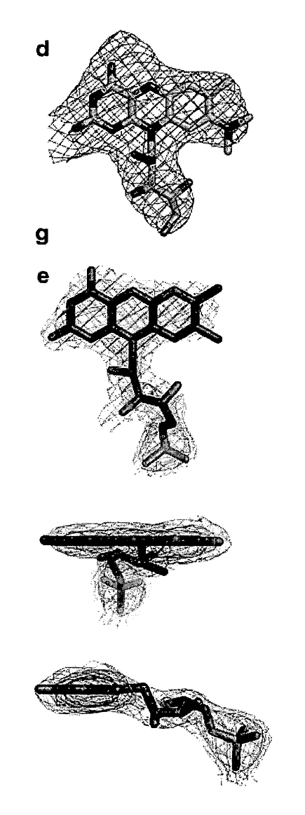

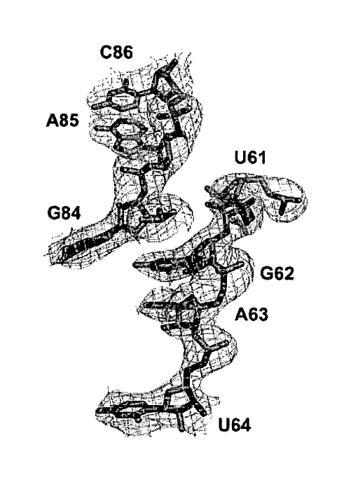

In FIG. 23. Interpretation of the electron density maps. a, b, Experimental electron density map (contoured at the 1 cr level) around the FMN binding pocket (a) and FMN (b) shown with the refined riboswitch model. The map was calculated using 3.0 A [Ir(NH3)6]3+ MAD data. c, d, 3.0 A omit Fo-Fc maps (2 cr level) calculated without riboflavin (c) and roseoflavin (d) and shown with the refined ligand models. e-g, Refined 2.95 A2Fo-Fc electron density map (blue, 1 cr level; red, 2 cr level) around FMN from the top (e), front (f) and side (g) views. FMN can undergo reversible redox interconversion between oxidized (FMN), semiquinone (FMNHe) and reduced (FMNH2) states. In the semiquinone and reduced forms, the isoalloxasine ring system is slightly bent along the N5-NIO axis 41. In the FMN riboswitch crystals, FMN ring system is planar consistent with the presence of the oxidized FMN. h, Refined 3.0 A 2Fo-Fc electron density map (1 cr level) in the region of the conformational changes observed in the riboflavin-riboswitch complex.

Recent studies have demonstrated slow kinetics of association and dissociation for the FMN-riboswitch complex, supportive of a kinetically driven riboswitch mechanism 30. These kinetic characteristics are consistent with the recognition principles identified in Applicants' three-dimensional structure. Indeed, riboswitch folding requires formation of multiple non-canonical and tertiary interactions and other conformational adjustments on FMN binding, which together may account for the slow association rate. Subsequent FMN release is likely to be slowed down due to envelopment of the ligand by the RNA. The propensity of other large riboswitches to function as kinetically driven genetic switches remains a challenging area for future exploration.

Lysine Riboswitches. To understand the molecular basis of amino acid recognition by riboswitches, here Applicants present the crystal structure of the 174-nucleotide sensing domain of the Thermotoga maritima lysine riboswitch in the 1.9 angstrom lysine-bound and 3.1 angstrom free states.

The riboswitch features an unusual and intricate architecture, involving three-helical and two-helical bundles connected by a compact five-helical junction and stabilized by various long-range tertiary interactions. Lysine interacts with the junctional core of the riboswitch and is specifically recognized through shape-complementarity within the elongated binding pocket and through several direct and K⁺¹-mediated hydrogen bonds to its charged ends. Applicants' structural and biochemical studies indicate preformation of the riboswitch scaffold and identify conformational changes associated with the formation of a stable lysine-bound state, which prevents alternative folding of the riboswitch and facilitates formation of downstream regulatory elements. Applicants have also determined several structures of the riboswitch bound to different lysine analogues 5, including antibiotics, in an effort to understand the ligand binding capabilities of the lysine riboswitch and understand the nature of antibiotic resistance. Applicants' results provide insights into a mechanism of lysine-riboswitch-dependent gene control at the molecular level, thereby contributing to continuing efforts at exploration of the pharmaceutical and biotechnological potential of riboswitches.

RNA sensors play a crucial part in many regulatory loops, owing to their capacity for directing gene expression in response to various stimuli in the absence of protein participation 6-8. Recent three-dimensional structures of a thermosensor 9, a metallosensor 10, a metabolite bound ribozyme 11,12 and riboswitches specific for purine nucleobases 13,14, and for co-enzymes thiamine pyrophosphate 15-17 and S-adenosylmethionine 18,19 have highlighted how each ribosensor uses unique structural features to sense its cognate stimulus. However, the molecular details of the organization of amino-acid-specific riboswitches, such as the lysine riboswitch, which efficiently discriminates against other free amino acids, their precursors and amino acids within a peptide context 1,2,5, remain obscure. The determination of the lysine riboswitch structure presents a considerable challenge, because of its large metabolite-sensing domain, predicted to form a five-way junction 1-3.

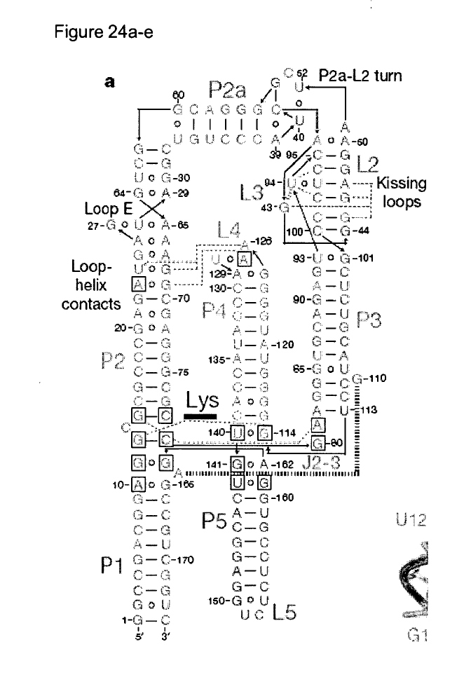

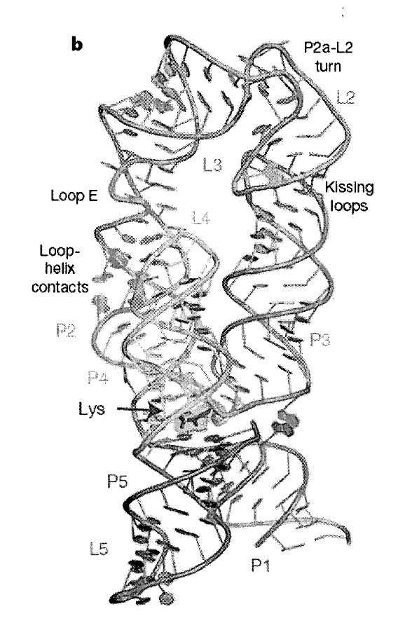

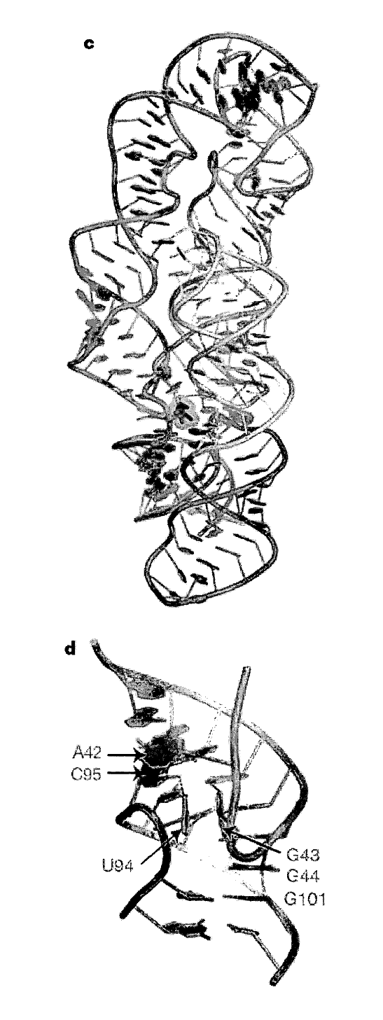

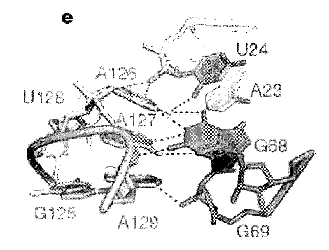

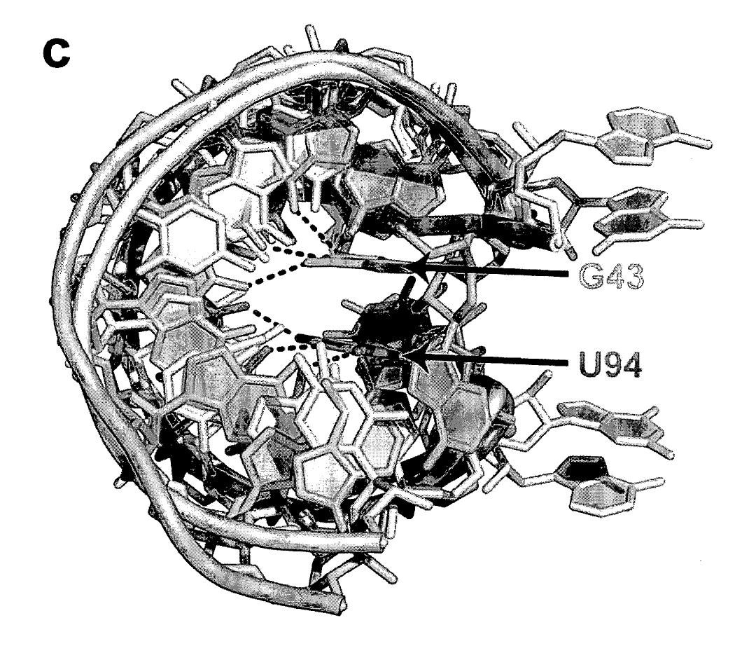

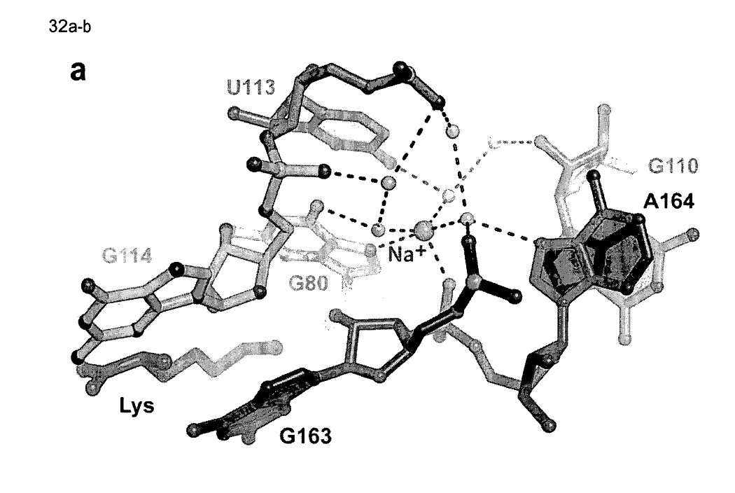

The T. maritima riboswitch is a typical lysine riboswitch 1-3,5 (Supplementary FIGS. 1 and 2a) which uses a transcriptional attenuation mechanism to repress the production of aspartate-semialdehyde dehydrogenase 3, which is involved in the synthesis of a precursor for methionine, threonine, lysine and diaminopimelate. The structure of the lysine-bound riboswitch domain, also known as an ‘L box’, features three-helical and two-helical bundles radiating from a compact five-helical junction (FIG. 24a-c and FIG. 29b-d) that contains lysine inserted into its core. The junction is organized on the basis of a modified four-way junction through colinear stacking of helices P1 and P2, and helices P4 and P5, positioned as two intersecting lines of an uneven letter ‘X’. In FIG. 29: Architecture and sequence conservation of the lysine riboswitch. a-b, Secondary structure schematics of the 172-nt B. subtilis (a) and the 174-nt T maritima (b) riboswitches used in the study. The schematics are based on the models from refs. 1-3,33. Stem-loops are color-coded similarly to those in FIG. 1. Red and blue nucleotides indicate invariant and conserved (>75% identity) nucleotides present in the T maritima riboswitch and III other lysine riboswitches from the Rfam database 34. The conservation analysis of the P2a-L2 tum and the L2-L3 kissing loops may not be accurate due to the length variations and possible sequence misalignments. Important long-range tertiary contacts are shown by dashed lines (blue lines for stacking and green lines for pairing). c, Schematics of the structure of the T maritima lysine riboswitch presented according with ref. 35. Invariant and conserved nucleotides, as well as color codes for the stem-loops are depicted as in (b). d, Overall lysine riboswitch structure in a ribbon representation. Invariant and conserved nucleotides, as well as color codes for the stem-loops are depicted as in (b).

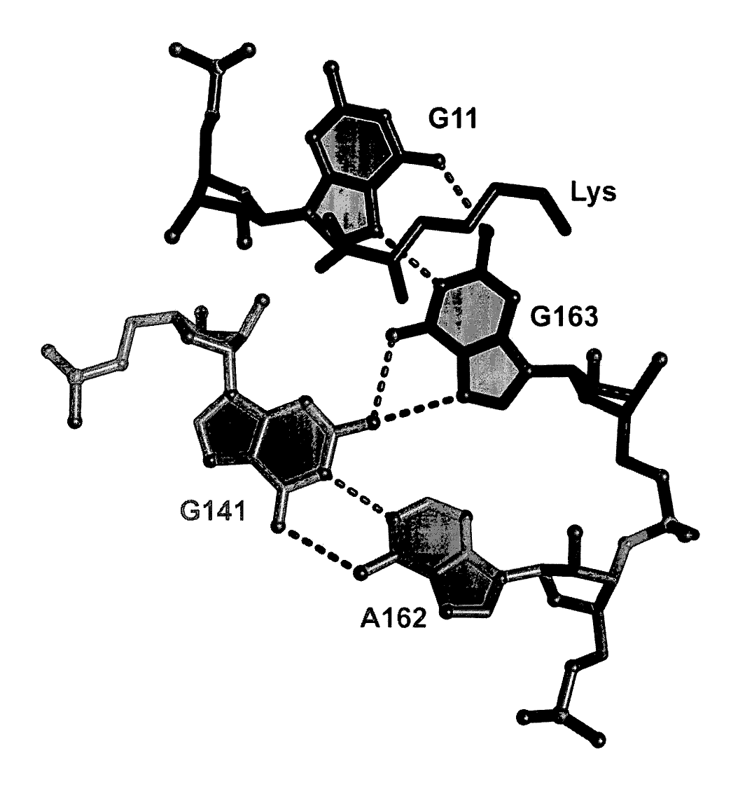

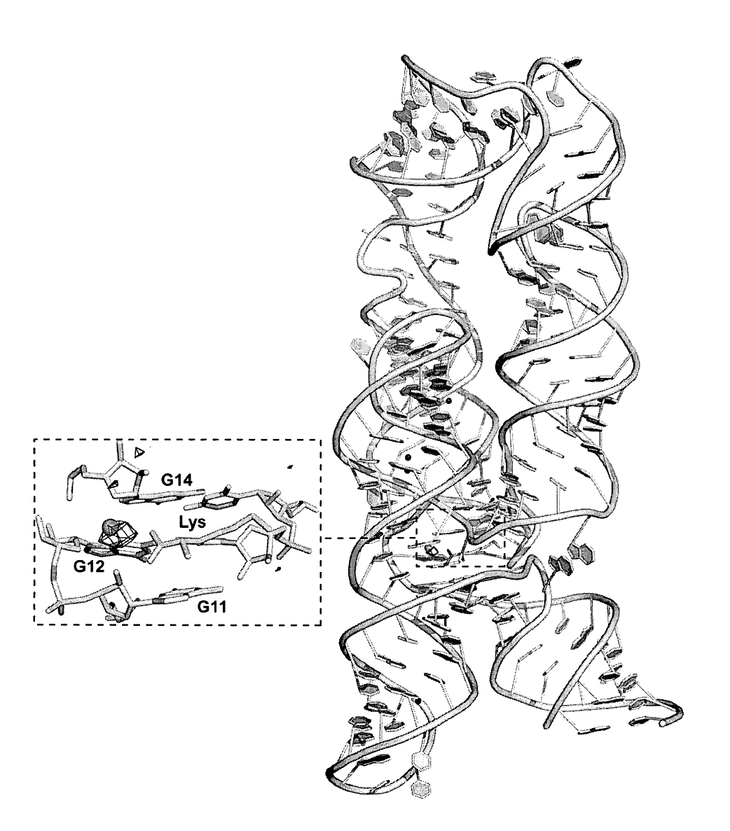

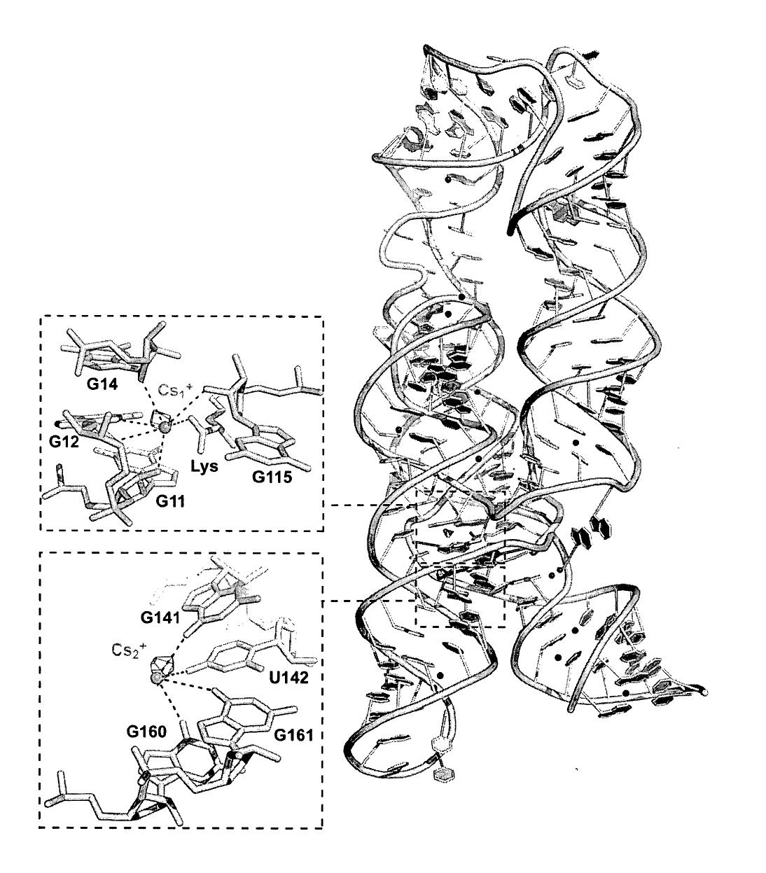

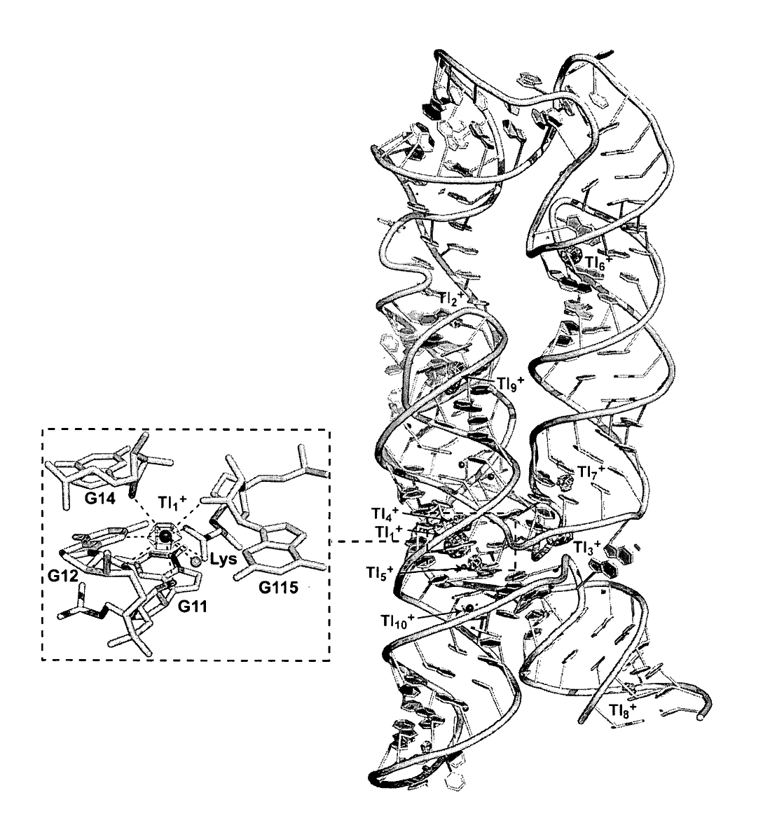

The P2-P2a-L2 stem-loop reverses its orientation through two turns important for riboswitch function 5,21: one of them adjacent to a loop E motif and the other centered on a turn that replaces the kink-turn motif found in other lysine riboswitches (FIG. 30). In FIG. 30: Structures and sequences of the RNA motifs from the T. maritima lysine riboswitch. a, Loop E motif. Left panel, sequences from the lysine riboswitch and 5S rRNA (PDB ID code IJJ2). Right panel, all-atom superposition of the loop E motif from the riboswitch (cyan) and 5S rRNA (brown). b, P2a-L2 turn from the lysine riboswitch, replacing the kink-turn motif found in other lysine riboswitches. Left panel: schematic representation of the turn. Watson-Crick and non-canonical base-pairs are shown by lines and circles, respectively. Stacking interactions are indicated by black rectangles. RNA backbone is depicted by solid colored lines. Right panel: the structure of the turn. Dashed lines depict hydrogen bonds. Note the syn conformation of C41. c, Kink-turn motifkt-7 from 16S rRNA24. Left panel: schematic representation of the motif. Right panel: superposition of the kt-7 turn (residues 77-82 and 93-99 in brown) (PDB ID code IJJ2) and the lysine riboswitch P2a-L2 turn (residues 37-42 in cyan and 50-56 in red). R.m.s.d. is 3.9 angstrom. Despite some similarities in the overall folds of these turns, the turn from the T maritima lysine riboswitch does not have the typical features of the canonical kink-turn motifs. d, A turn from the P4-P6 domain of group I introns (PDB ID code 1JID)36. Left panel: schematic representation of the motif. Right panel: superposition of the P4-P6 turn (residues 119-123, 126, and 196-202, in green) and the P2a-L2 turn (residues as in (c)). R.m.s.d. is 3.7 angstrom. Stems P2 and P3 are aligned by an unusual kissing-loop complex between loops L2 and L3 (FIG. 24d and FIG. 31), whereas parallel stems P2 and P4 are anchored by a conserved loop (L4)-helix (P2) interaction (FIG. 24e). In FIG. 24: Overall structure and long-range tertiary interactions of the lysine-bound T. maritima riboswitch. a, Schematic of the riboswitch fold observed in the crystal structure of the complex. The bound lysine is in red. The RNA domains are depicted in colors used for subsequent figures. Base specific tertiary contacts and long-range stacking interactions are shown as thin green and thick blue dashed lines, respectively. Nucleotides invariant in known lysine riboswitches are boxed. b, c, Overall lysine riboswitch structure in a ribbon representation showing front (b) and rotated by ,60 u (c) views. d, The L2-L3 kissing loop interaction is formed by six base pairs, supplemented by interstrand stacking interactions between A42 and C95, G43 and U94, and G44 and G101. Hydrogen bonds between interstrand base pairs and orthogonally aligned G43 and U94 bases are depicted by dashed lines. e, The L4-loop-P2-helix interaction formed by an insertion of the A126-A127-A129 stack of L4 into the RNA groove of P2 distorted by noncanonical base pairs. In FIG. 31: Structural details of the L2-L3 loop interaction. a, Hydrogen bonding of G43 and U94 with base pairs of the inter-loop helix. G43 and U94 are oriented perpendicular to other bases in the loops, positioned inwards toward the RNA groove, and involved in hydrogen bonding with the G45-G46-A47 and C96-U97-C98 segments, respectively. The G431U94 element, absent in other kissing loop complexes, zippers up the entire motif and likely contributes to the overall stability of the riboswitch conformation. band c, Side and top views of the all-atom superposition of the L2-L3 kissing loops from the lysine riboswitch (residues 44-49 in cyan and 95-100 in orange) and the kissing loop complex from the HIV-1 dimerization initiation site (DIS) (residues 10-15, chains C and D in grey) (PDB ID code 1K9W)37. R.m.s.d. of the superposed structures is 1.5 angstrom. The lysine riboswitch loops are closed by the non-canonical A42•A50 base pair, which is part of the P2a-L2 tum, and the U93•G101 base pair. Six canonical base pairs forming the intra-loop helix are superposed well between both structures. In the DIS structure, however, the residues corresponding to the G431U94 element are oriented outwards.

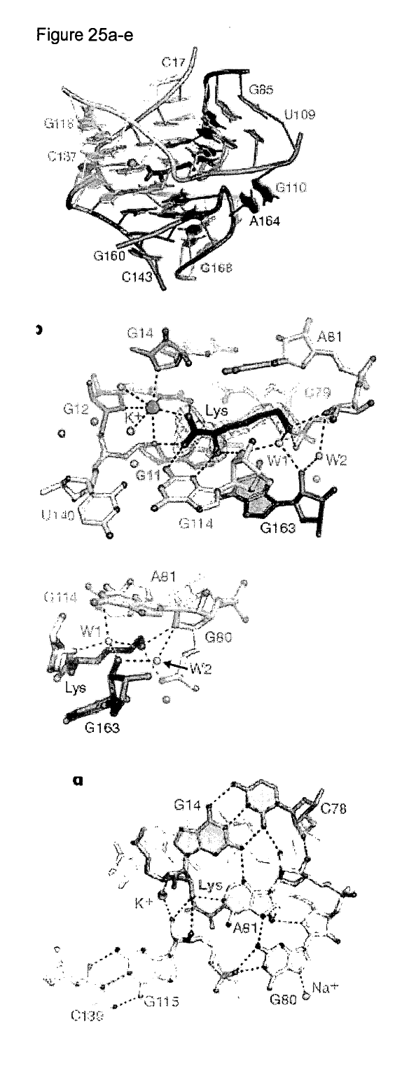

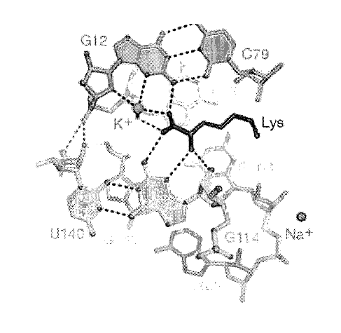

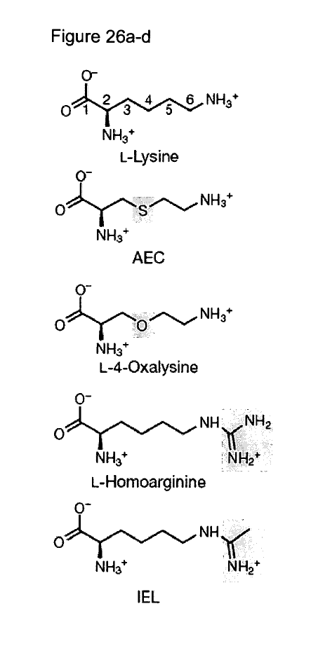

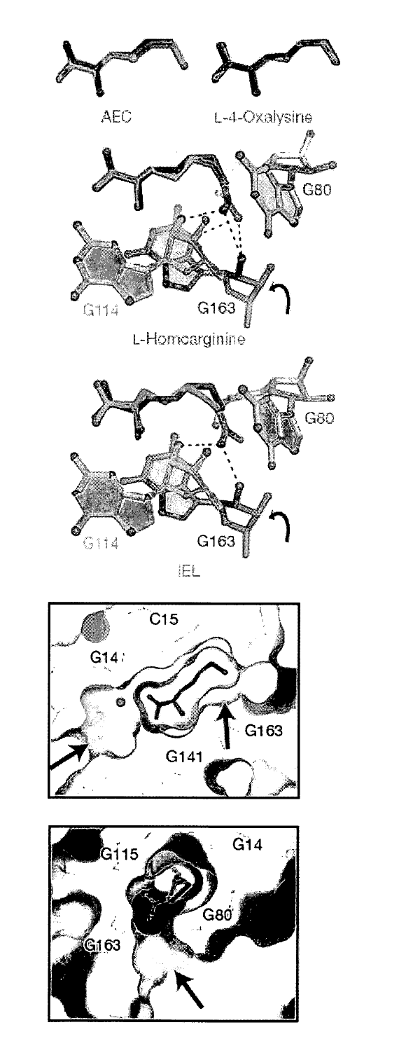

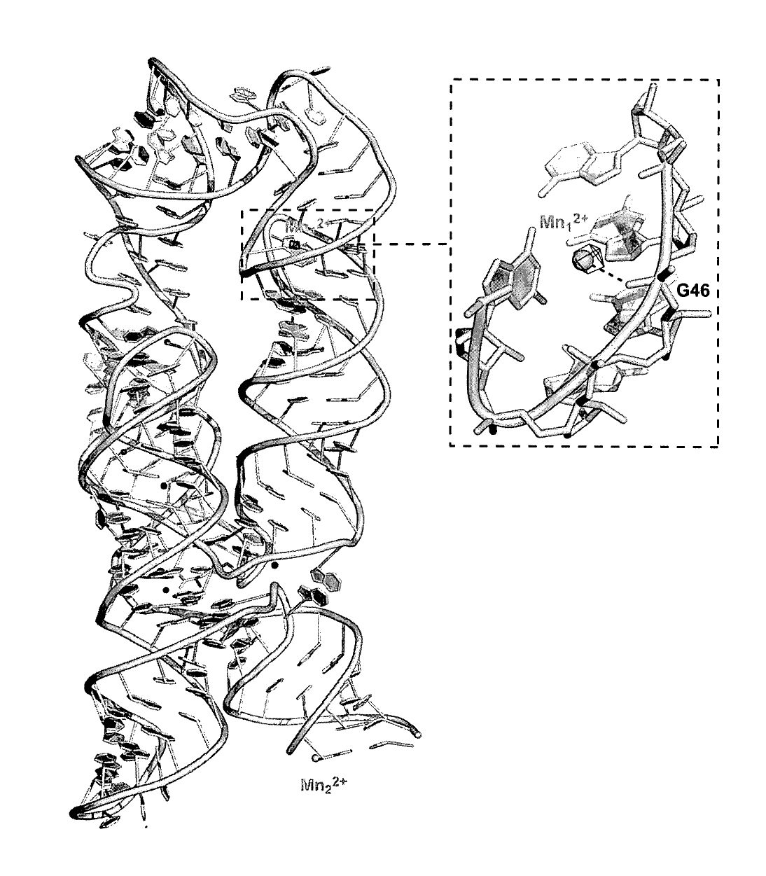

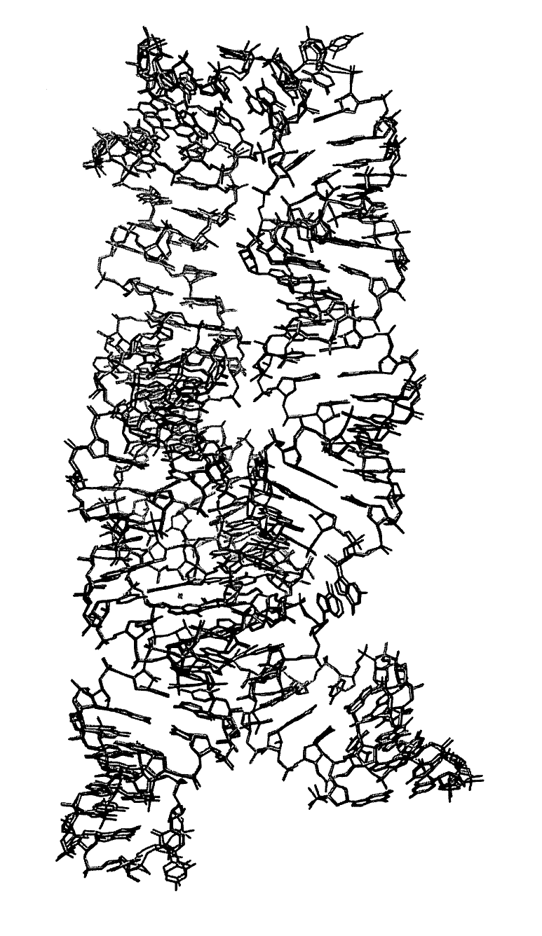

The five-helical junction contains three layers of nucleotides, each composed of two interacting base pairs, organized around the centrally positioned lysine which fits into a tight pocket and is specifically recognized by its charged ends (FIG. 25a-c). This lysine-bound pocket architecture (FIG. 25) is also retained in lysine analogue complexes (FIG. 26) outlined later. The top layer, composed of G14-C78 and G115-C139 base pairs (FIG. 25d), is stabilized through ribose zipper and type I A-minor triple interactions with invariant A81, which in turn is paired with Na⁺¹-bound G80 (FIG. 25d and FIG. 32a). The middle layer, containing G12-C79 and G114NU140 base pairs, forms specific interactions with the bound lysine (FIG. 25e). In FIG. 25: Structure and interactions in the junctional region of the lysine riboswitch. a, Stereo view of the junction with bound lysine. Green sphere depicts a K⁺¹ cation. b, Details of riboswitch lysine interactions. Lysine is positioned within the omit Fo2Fc electron density map contoured at 3.5s level. Water molecules are shown as light blue spheres. K⁺¹ cation coordination and hydrogen bonds are depicted by dashed lines. c, Direct and water-mediated interactions involving e-ammonium group of lysine. d, e, Interactions in the top (d) and middle (e) junctional layers. In FIG. 32: Structural details of junction organization. a, Na+- and water-mediated interactions that contribute to the anchoring of G80 and the stabilization of the segments from helices PI (in magenta) and P3 (in orange) adjacent to the junction. b, Formation of a purine quartet by interactions between the conserved non-canonical G11-G 163 and G141-A162 pairs. Note the syn conformation of G11.

Both the carboxylate and ammonium groups of the lysine ‘main chain’ segment are hydrogen-bonded to the minor groove edges of purine bases and sugar 29-OH groups (FIG. 25b, e), whereas the e-ammonium protons of the side chain form hydrogen bonds with a non-bridging phosphate oxygen, a sugar ring oxygen and a tightly bound water molecule, W1 (FIG. 25c). In addition, lysine is sandwiched between the A81 base (FIG. 25d) and the G11NG163 base pair from the bottom junctional layer (FIG. 25e and FIG. 32b), thereby stabilizing the top of the P1 helix which is necessary for gene expression control. The bound lysine facilitates the holding together of the stacked P1-P2 and P4-P5 helical segments, and contributes to the positioning of the P3 helix by locking up G80, which is placed over the e-ammonium group of lysine, and stacks with the A82-U113 base pair of P3 (FIG. 25a). This stable junctional conformation is further reinforced by a tertiary stacking interaction between G110 and A164 (FIG. 25a) characteristic for riboswitches from thermophiles, as well as other interhelical contacts (FIGS. 1b, c and 2a).

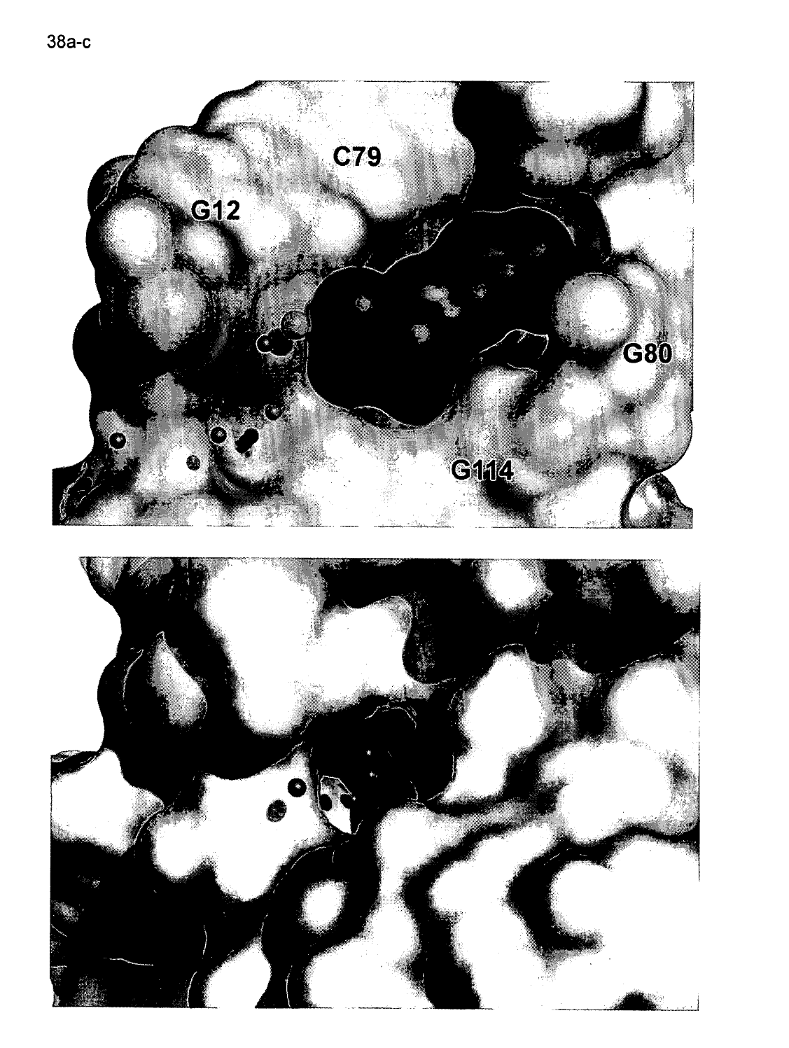

A notable feature of the lysine-binding pocket is a K⁺¹ cation (FIG. 25b), which binds a carboxyl oxygen of lysine and zippers up the junction using several coordination bonds. The K⁺¹ cation is directly observed on the anomalous map (FIG. 33) and is replaceable by its mimics, Cs⁺¹ and Ti⁺¹, but not by Mn⁺² (FIGS. 30-32), a mimic of Mg⁺². In FIG. 33: K⁺ binding site in lysine riboswitch. The refined lysine riboswitch structure (2.9 AK+ anomalous data) superposed with the anomalous map (pink) contoured at the 4.5 cr level and at the 3.5 cr level in the zoomed-in view. The zoomed-in view is slightly rotated with respect to the overall view. Na+ cations and the lysine-bound K+ cation are shown as violet and green spheres, respectively. Two more K+ cations found in the 1.9 A native structure are not shown in this figure. These K+ cations have weak peaks (the 3.1 and 3.5 cr levels, practically noise levels) on the 2.9 A anomalous map and do not have positive electron density on the 2.9 A2Fo-Fc map.

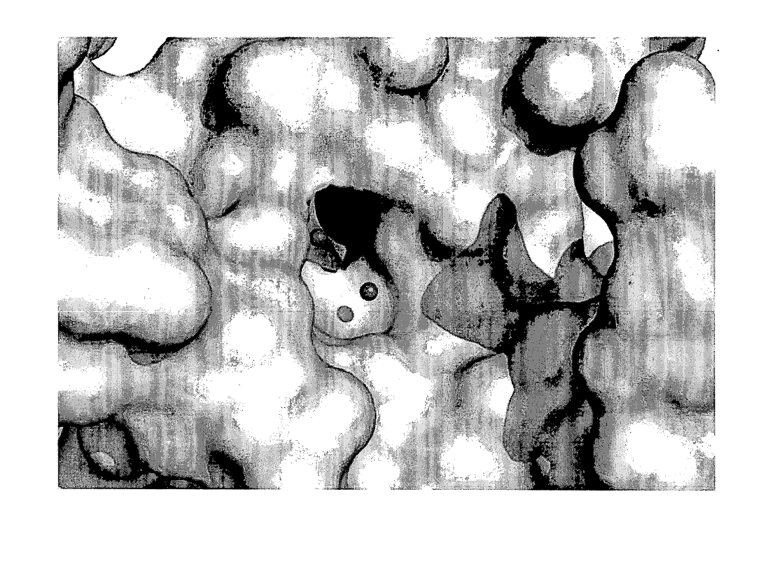

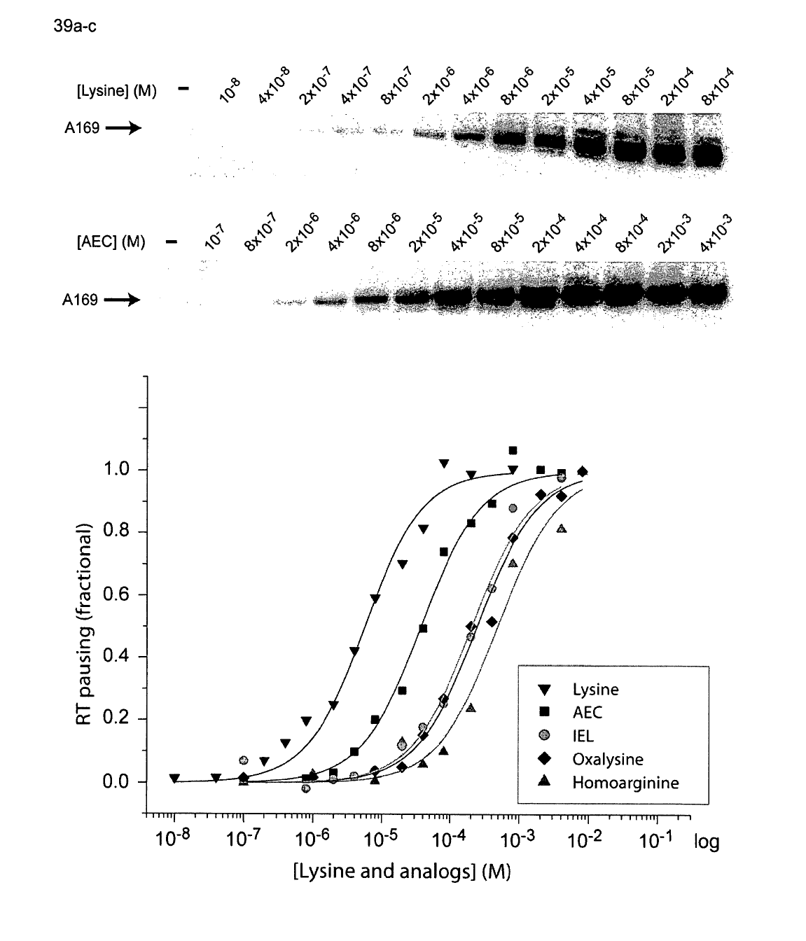

In FIG. 34: Cs+ binding sites in the lysine riboswitch. The refined lysine riboswitch structure (Cs+ anomalous data) superposed with anomalous map (pink) contoured at the 4.5 0: level. Cs+ and Na+ cations are shown as light green and violet spheres, respectively. Two cesium cations, CSj+ (˜5 CJ level) and CS2+ (˜5.5 CJ level), have been identified in the map. CSj+ replaces the K⁺ cation coordinated with the bound lysine. CS2+ binds in a site that, in the high resolution native structure, is occupied by an elongated electron density, modeled as a small segment of a largely unstructured PEG molecule.

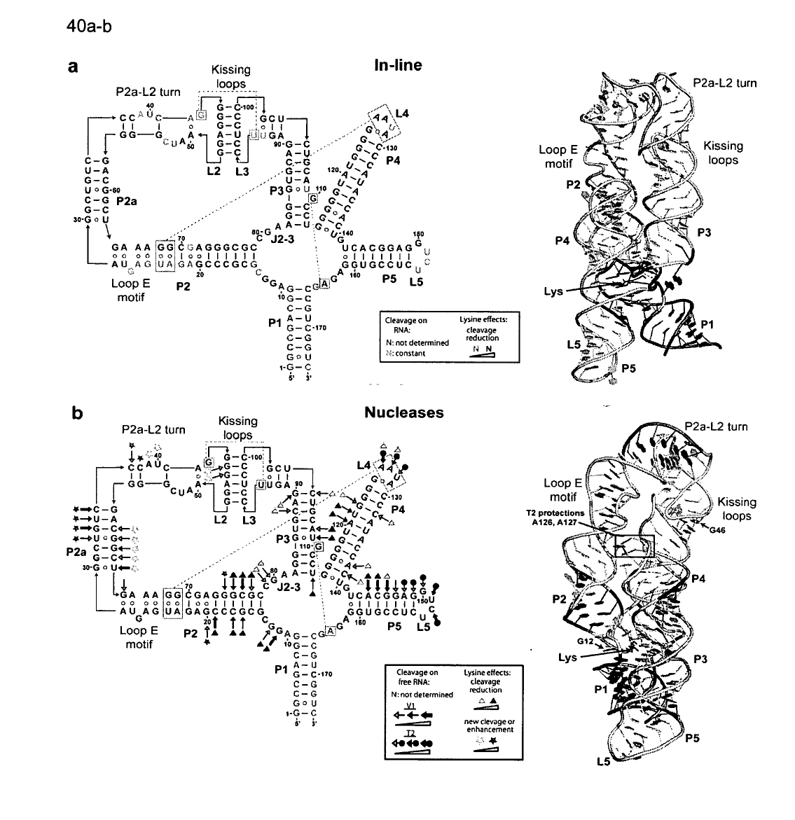

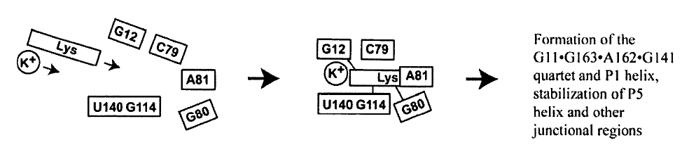

In FIG. 35: TI+ binding sites in the lysine riboswitch. The refined lysine riboswitch model (TI+ anomalous data) superposed with the anomalous map (light pink) contoured at the 3.5 q level and the 30 (J level in the zoomed-in view. TI+ and Na+ cations are shown as brown and violet spheres, respectively. The water molecule is in light blue. Ten TI+ cations, TIl+ to TIlQ+ (˜4-42 (J level) have been identified in the map. T19+ is split into two sites. TIl+, corresponding to the strongest anomalous peak (˜42 (J level), replaces the lysine-coordinated K+ cation. T12+ and Tb+ replace Na+ cations, Th+ and T18+ replace water molecules, and TIlQ+ replaces the segment of a PEG molecule, while the other TI+ cations bind new sites typically in close proximity to the water molecules identified in the native high-resolution structure.

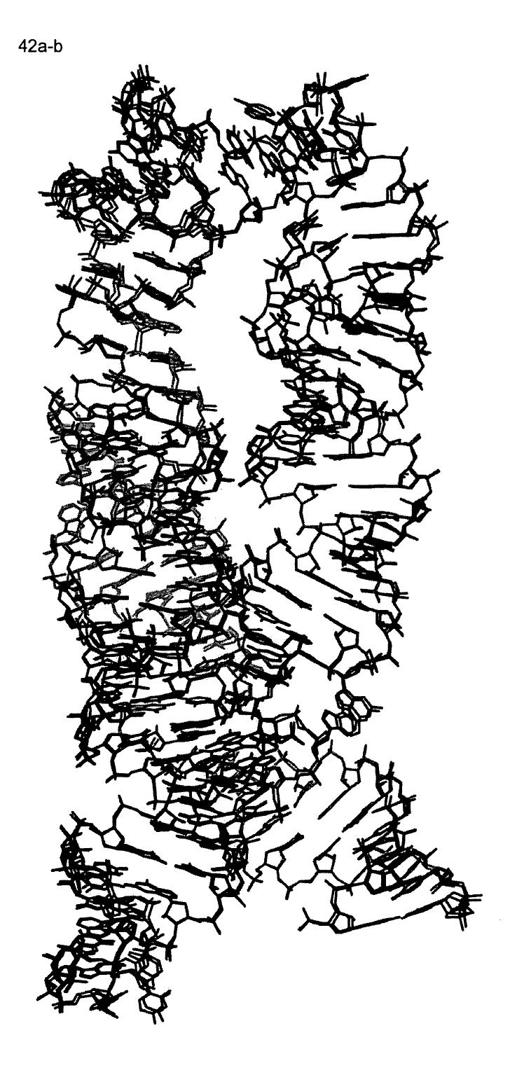

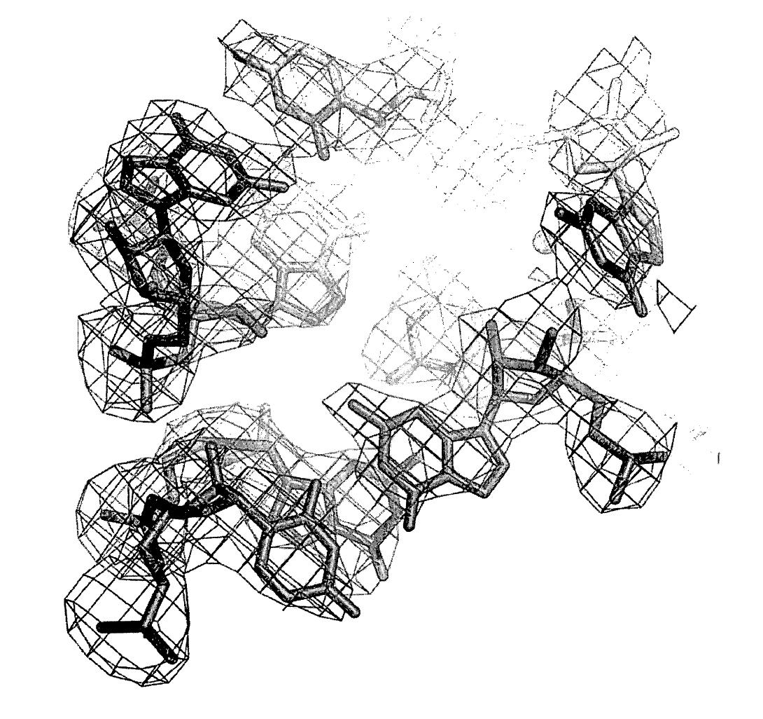

In FIG. 36: Mn2+ binding sites in the lysine riboswitch. The refined lysine riboswitch model (Mn2+ anomalous data) superposed with the anomalous map (pink) contoured at the 4.0 a: level. Mn2+, K+ and Na+ cations are shown as cyan, green and violet spheres, respectively. To compensate for the possible chelating effect of Na-citrate in the crystallization solution, Mn2+ concentration was increased up to 50 mM during soaking. Two manganese cations, Mn12+ (˜5 cr level) and Mn22+ (˜3 cr level, practically a noise level), have been identified in the map. Mn12+ is coordinated with the non-bridging phosphate oxygen and occupies the position of a water molecule in the high resolution native structure. Due to the lower resolution of this structure (2.7 A), Mn12+-coordinated water molecules may not be seen in the map. Mn22+ binds to the Sf-triphosphate moiety. No anomalous signal has been observed at the position occupied by the lysine-bound K+.

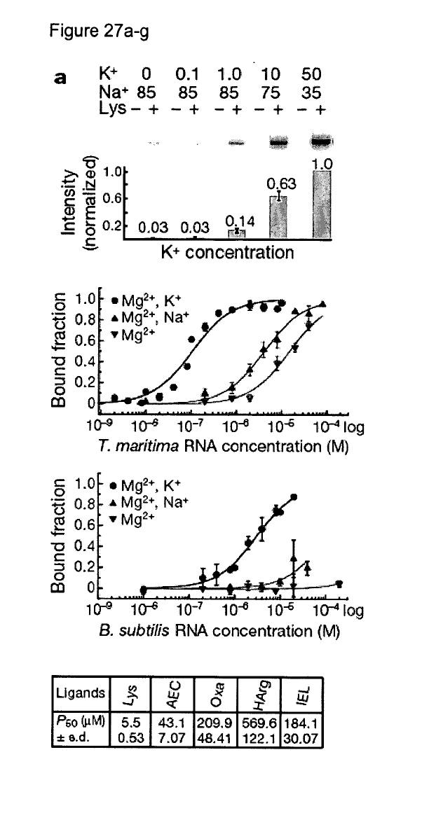

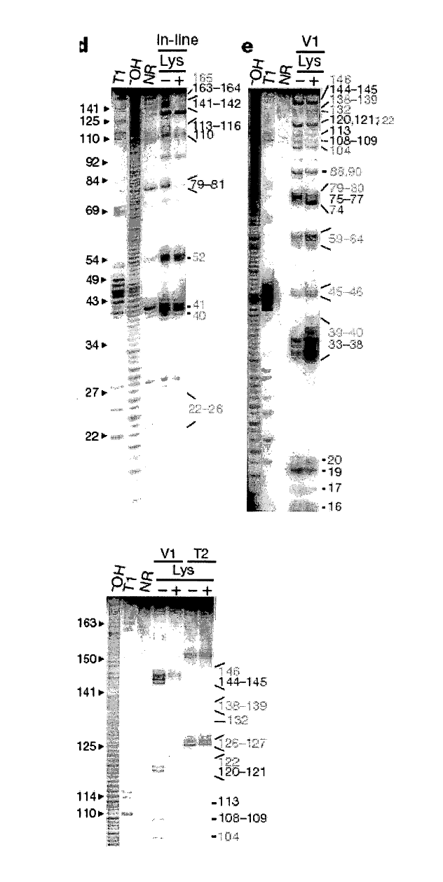

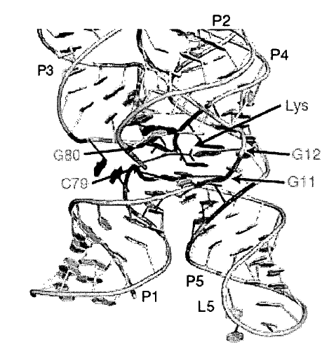

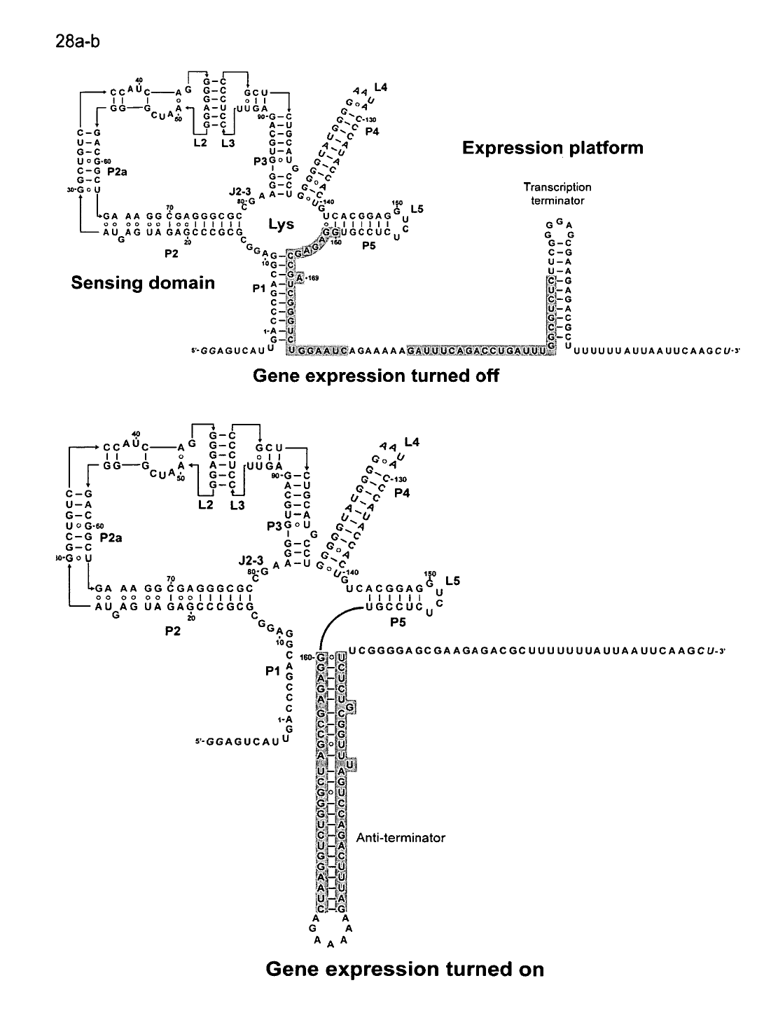

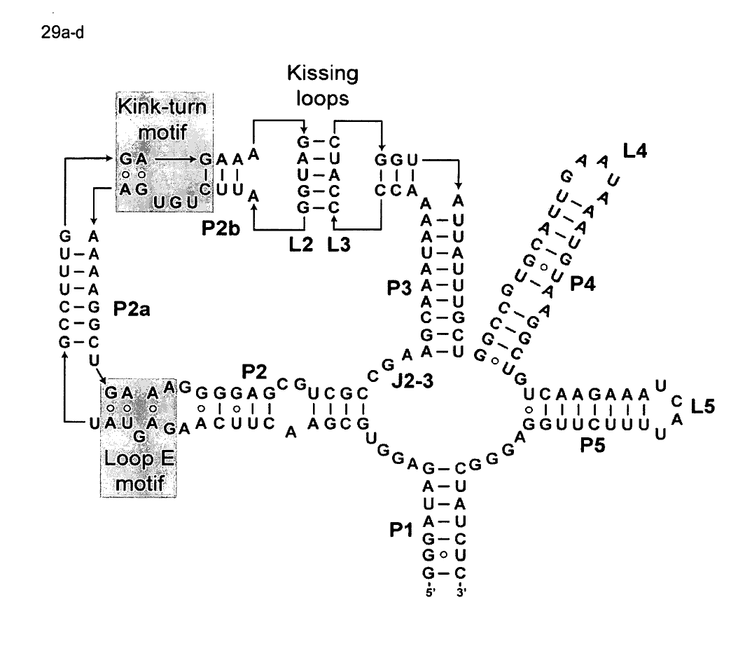

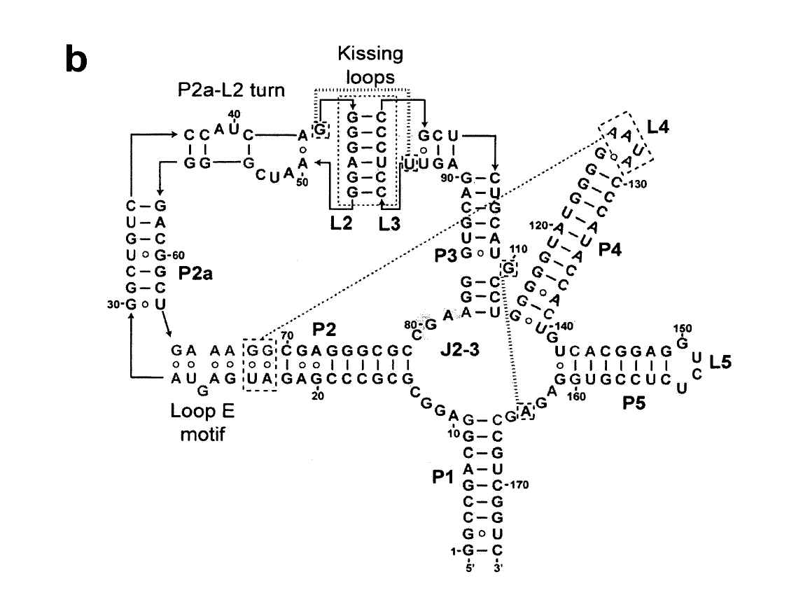

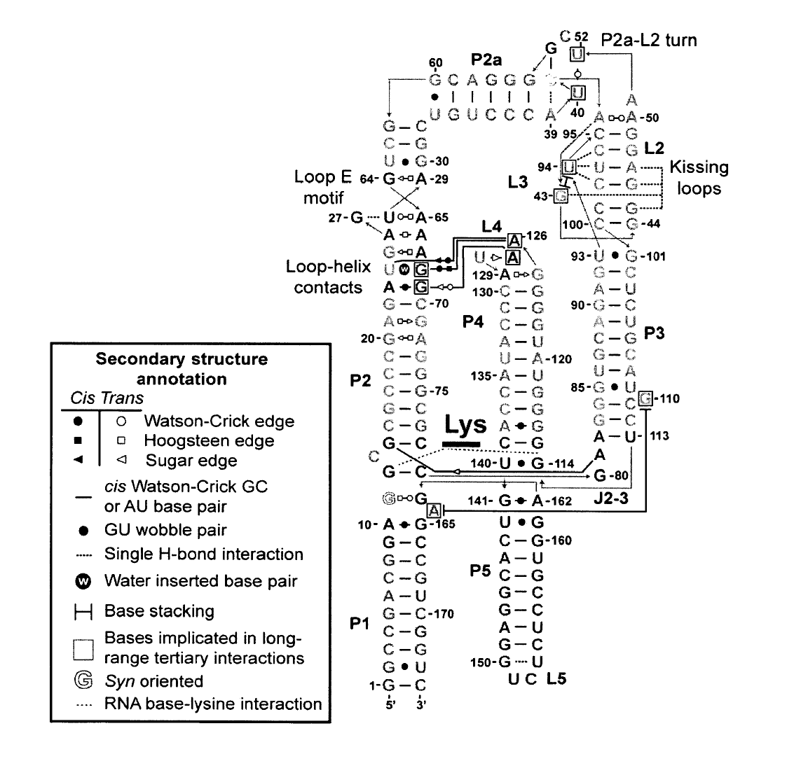

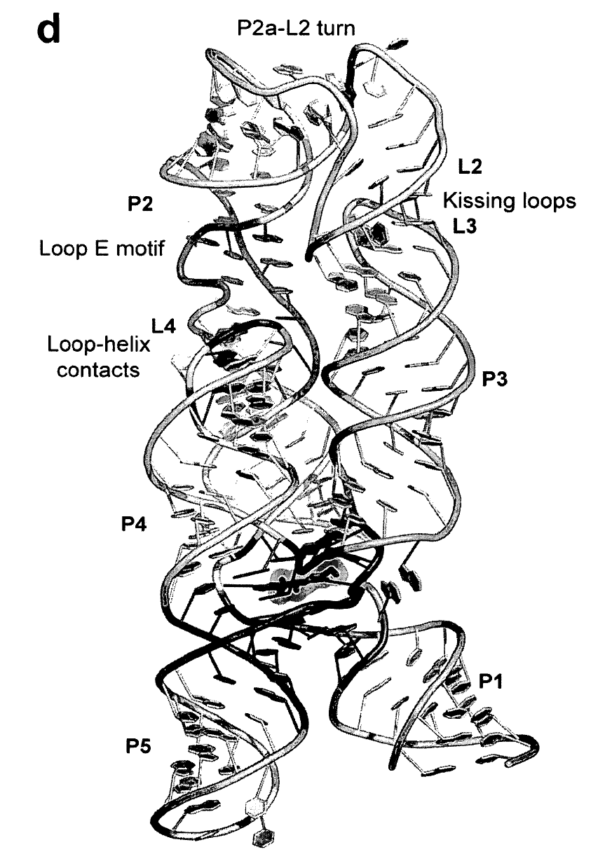

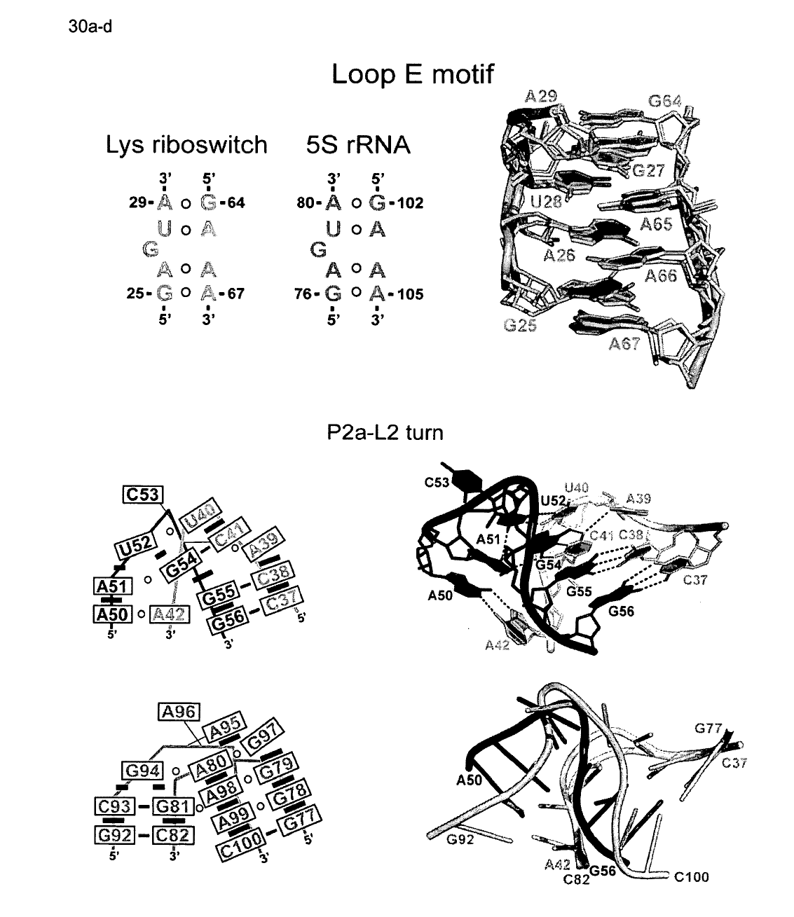

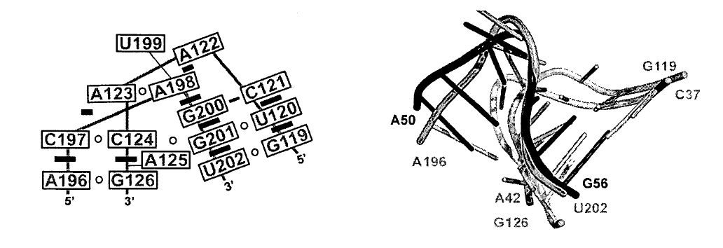

The importance of K⁺¹ for lysine binding has been demonstrated in primer extension experiments. In the presence of lysine and at physiological concentration of K⁺¹, reverse transcriptase pauses before the junction at A169, reflecting the formation of a stable junctional conformation (FIG. 27a, lanes 8, 10). The pause decreases 33-fold after the replacement of K⁺¹ by Na⁺¹ (FIG. 27a, lanes 2, 4), suggesting that there is either reduced stability or failure to generate a junction competent for lysine recognition under K⁺¹-free conditions. These results have been supported by equilibrium dialysis experiments with T. maritima and Bacillus subtilis riboswitches. In both cases, lysine binding affinity significantly decreased when K⁺¹ was omitted or replaced by Na⁺¹. (FIG. 27b). Note that lysine binds better to a riboswitch from a thermophile than from a mesophile. In FIG. 27: Probing lysine riboswitch tertiary structure. a, Primer extension analysis at various K⁺¹ concentrations. 32P-labelled oligonucleotide was annealed to the 39 end of 265-nucleotide T. maritima RNA (1 mM) and extended by reverse transcriptase in the presence of 2 mM MgCl₂, at indicated concentrations of monovalent cations (in mM), and with or without a tenfold excess of lysine over

RNA. b, Lysine binding affinity measured by equilibrium dialysis for riboswitches from T. maritima (top) and B. subtilis (bottom). Mg⁺², K⁺¹ and Na⁺¹ concentrations are 20, 100 and 100 mM, respectively. The dissociation constants (mean6s.d., mM; n52-4) are: T. maritima, 0.1060.03 (Mg⁺²K⁺¹), 4.1460.67 (Mg⁺²Na⁺¹), 15.9360.09 (Mg⁺²); B. subtilis, 2.9560.30 (Mg⁺²K⁺¹). c, The apparent ligand concentration at which reverse transcriptase pausing is half-maximally attained (P50; n52) in the primer extension experiments. HArg, L-homoarginine; IEL, iminoethyl-L-lysine; Lys, L-lysine; Oxa, L-4-oxalysine. d, In-line probing of 59 32P-labelled T. maritima 174-nucleotide RNA (1.6 nM) in the absence and presence of lysine (1.1 mM). T1 and 2OH designate RNase T1 and alkaline ladders, respectively. NR, no reaction. Strong and weak cleavage reductions are shown in red and pink colors, respectively. e, f, Probing of 174-nucleotide RNA (1 mM) by V1 and T2 nucleases with or without a tenfold excess of lysine. Weak and strong cleavage enhancements are shown in light and dark green, respectively, in e. g, Strong lysine-induced cleavage reductions are color coded in the riboswitch structure. Light orange, green and red are reductions identified in ref. 1 using B. subtilis RNA with a short P1 helix, overlapping reductions of the present study and in ref. 1, and extra reductions found in the present study, respectively.

The preference of K⁺¹ for binding to the negatively charged carboxylate group contrasts with Mg⁺²-mediated phosphate recognition in other ribosensors 11,12,15,16 and might also be a characteristic for other amino-acid-specific riboswitches. Because more than 20 lysine-binding proteins (listed in the Protein Data Bank) do not use cations to mediate lysine recognition, this feature is probably unique to RNA, given that it lacks the positively-charged side chains found in proteins.

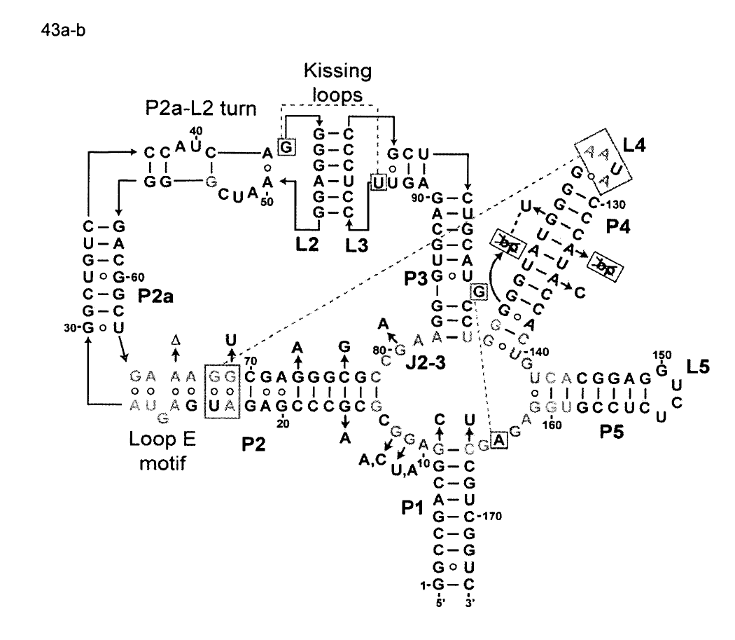

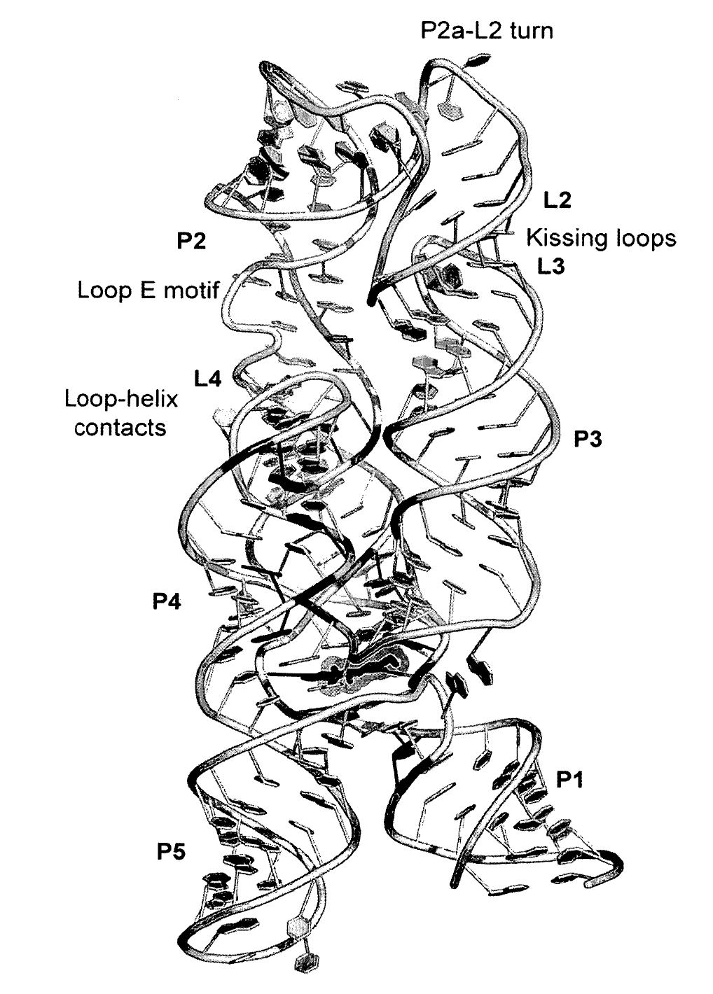

Folding of most RNAs, including the B. subtilis lysine riboswitch 21, requires Mg⁺². However, the crystals of the T. maritima lysine riboswitch can be grown in the absence of Mg⁺² (FIG. 37). Moreover, a Mn⁺² soak does not replace cations in the structure grown with Mg⁺², and, on the basis of the coordination distances, most of these cations can be assigned as Na⁺¹. These results indicate that the L box from a thermophile does not critically depend on Mg⁺², the function of which can be co-opted by monovalent cations. In FIG. 37: Comparison of the lysine riboswitch structures obtained from the crystals grown in the absence and presence of Mg²⁺. All-atom superposition of the lysine riboswitch structures crystallized in the presence (red) and absence (blue) of Mg²⁺. R.m.s.d. is 0.33 A.

The identification of antibiotic-resistance mutations 27,28 in the lysine riboswitch 1,2, together with the demonstration of a direct interaction between the riboswitch and lysine-like antibacterial compounds 1,2,5, suggest that riboswitch targeting, along with other processes 29, is an important component of the antibiotic activity.

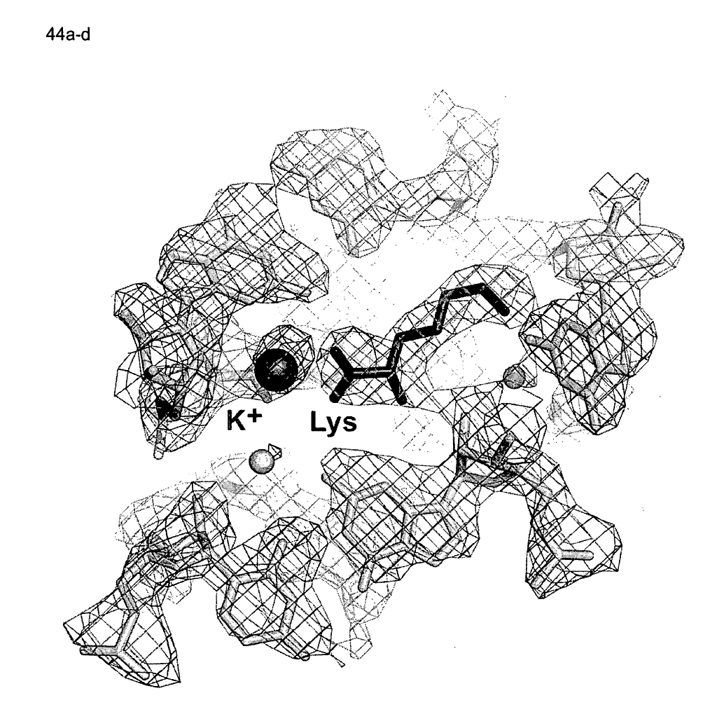

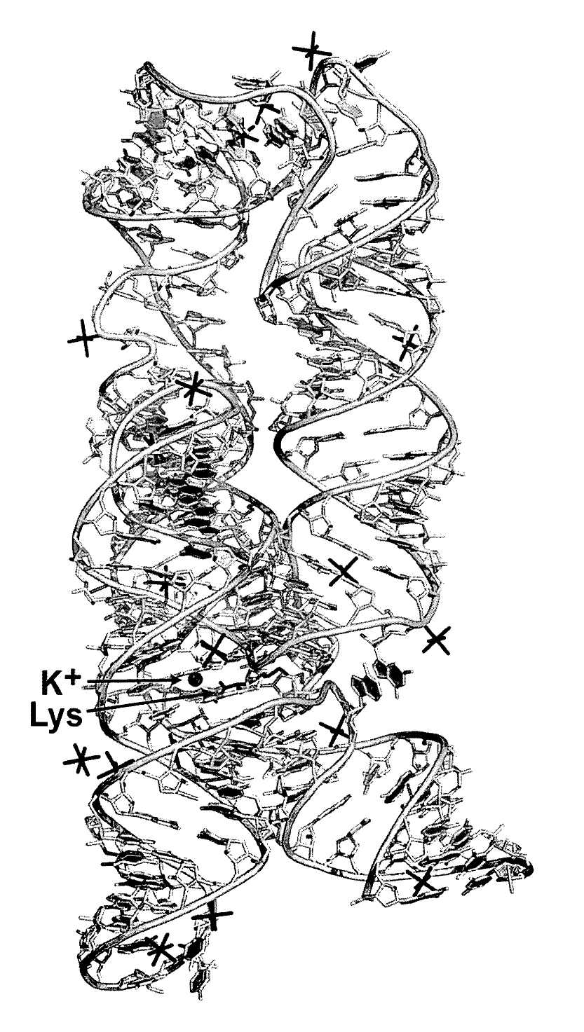

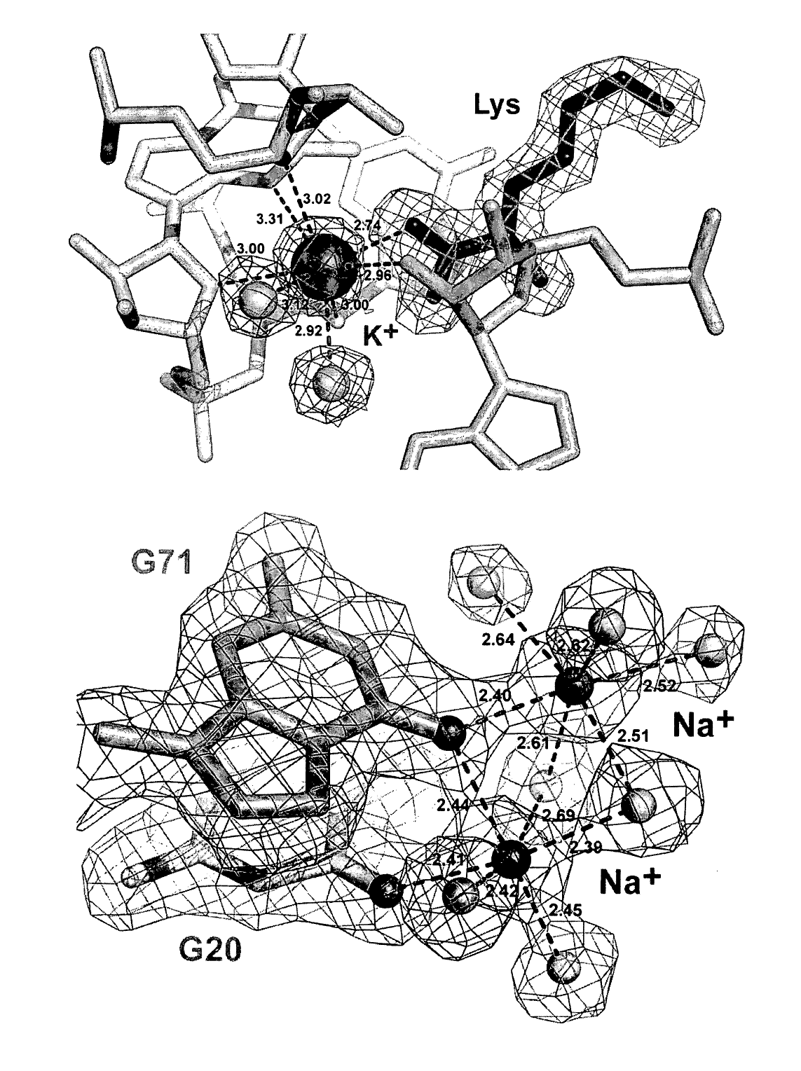

To understand the molecular basis of antibiotic resistance and explore the pharmaceutical potential of the lysine riboswitch, Applicants have determined structures of the riboswitch bound to antibacterial compounds S-(2-aminoethyl)-L-cysteine (AEC) and L-4-oxalysine-5, which contain sulphur and oxygen at position C4, respectively (FIG. 26a). In FIG. 26: Interactions of lysine analogues with the riboswitch. a, Chemical structures of lysine and its analogues. b, Conformation and interactions of lysine analogues with the riboswitch. Lysine (red) and analogues (cyan) are superposed. Interactions between riboswitch and lysine analogues should be compared with lysine recognition in FIG. 2b. c, Cross-section through the surface view of the lysine-binding pocket showing the opening next to the carboxyl group of lysine (red arrow) and the free space next to the C4 atom (blue arrow). d, Cross-section through the homoarginine-riboswitch complex showing the opening (red arrow) next to the guanidinium group. Because the pocket has a small cavity between the C4 and N7 positions of bound lysine (FIG. 26c and FIG. 38a), both C4-substituted analogues can be placed within the pocket in a manner similar to bound lysine (FIG. 26b, top panel), suggesting the potential for incorporation of even larger C4-substituents. Despite similar placement within the pocket, primer extension assays suggest that there is weaker binding of AEC and L-4-oxalysine to the riboswitch (FIG. 27c and FIG. 39), possibly due to an increased electronegativity of substituents at the C4 position. In FIG. 38: Surface views of the riboswitch bound to lysine. a, Lysine (red color) shown inside of the binding pocket. The view is similar to the view in FIG. 2c. b, The view of the opening next to the carboxylate group of bound lysine; c, The view of the opening next to the £-ammonium group of the bound lysine. Positions of the lysine-bound K+ cation and water molecules are indicated by green and blue spheres, respectively. Note that the colored spheres representing the cation and water molecules should be distinguished from their shadows.

In FIG. 39: Interactions of lysine and its analogs with the T. maritima lysine riboswitch studied by primer extension. a and b, Representative gels of riboswitch titration by lysine (a) and AEC (b). Reverse transcriptase (RT) pauses three nucleotides prior to the j unction at A169 in the context of the full-length riboswitch if the riboswitch-binding ligand is present in the reaction mixture. c, Representative titration experiments used for calculations of the ligand concentration (FIG. 4c) at which RT pausing is half-maximally attained. The experimental data points for lysine and AEC are from panels (a) and (b). The data were fitted to equation (1), which is based on the equation describing a bimolecular equilibrium, by the nonlinear least squares analysis. In the equation

$$θ = \frac{( {L}_{0} + {N}_{0} + {P}_{50} ) - \sqrt{{( {L}_{0} + {N}_{0} + {P}_{50} )}^{2} - 4 {L}_{0} {N}_{0}}}{2 {N}_{0}}$$

θ is the fraction of RT pausing, No and Lo are RNA and ligand concentrations, respectively, and P50 is the apparent ligand concentration at which RT pausing is half-maximally attained. The P50 value for the lysine-induced RT pausing 5.50±0.53/−1M (mean±s.d.) (n=2) is similar to the Kd value 4.03±0.34/−1M (mean±s.d.) (n=2) determined by the equilibrium dialysis assay. The relative differences between the P50 values for lysine and its analogs are in overall agreement with the relative differences obtained from the Kd values for lysine and its analogs determined for the B. subtilis lysine riboswitch in ref. 5.

Next, Applicants determined the structures with lysine analogues L-homoarginine and N6-1-iminoethyl-L-lysine, where the ammonium group of lysine is replaced by a guanidinium group and its methyl-substituted variant, respectively (FIG. 26a)5. In these structures (FIG. 26b), the side chains of lysine analogues are slightly shifted to provide better stacking interactions with the G80 base. The nitrogen atoms of the guanidinium-like extensions replace water molecules W1 and W2, found in the lysine complex (FIG. 2b). The G163 sugar is slightly rotated, so that the hydrogen bond pattern of W1 is retained by both ligands, whereas L-homoarginine forms extra hydrogen bonds with G163. Although most RNA-ligand contacts are preserved, both analogues demonstrate weaker interactions than lysine in primer extension (FIG. 27c and FIG. 39) and in-line probing 5 experiments, emphasizing the fine structural complementarity between the e-ammonium group of lysine and RNA.

The lysine-binding pocket has two openings, which could be exploited for the design of next generation lysine-like analogues (FIG. 26c, d and Supplementary FIG. 11b, c). One of the openings could accommodate modifications or extensions of the carboxylate group, possibly by substituting the K⁺¹ cation. The other smaller opening could allow extensions from N9 of L-homoarginine and iminoethyl-L-lysine. The analogue-bound structures and the biochemical data 5 indicate that the lysine-binding pocket is rather rigid, and only accommodates compounds which can sterically fit the pocket. Therefore, lysines in a polypeptide chain and branched amino acids are not recognized by the riboswitch. The lysine riboswitch also discriminates against smaller amino acids that fit into the pocket (data not shown) but are unable to make essential intermolecular contacts in the vicinity of G80.

To gain insights into lysine-induced conformational rearrangements of the riboswitch in solution, Applicants performed footprinting experiments using in-line probing, specific for flexible RNA regions, and cleavage by the nucleases T2 (single-stranded RNA) and V1 (paired and stacked regions). As in B. subtilis 1,5, the transition from the free to the lysine-bound states of the T. maritima riboswitch is accompanied by conformational changes within the junctional core, detected as strong cleavage reductions in both in-line and V1 probing (FIG. 27d-f and FIG. 40). However, the kissing loop complex is probably preformed in the free riboswitch 5,21, as evident from the absence of cleavages in in-line 1,5 (FIGS. 27d) and T2 probing (FIG. 40b), coupled with V1 cleavages at nucleotides 45-46 (FIG. 27e), which are only weakly enhanced by lysine binding. In FIG. 40: Footprinting of the T. maritima lysine riboswitch using in-line probing (a) and nuclease footprinting (b). Cleavages (in-line probing only) and lysine-induced cleavage reductions and enhancements are shown in the three-dimensional structures according to the color coding of the corresponding schematics. The assessment of cleavages near the RNA termini was not reliable and, therefore, was excluded from analysis (blue nucleotides). Most of the nucleotides demonstrating constant scission in the in-line probing assay correspond to the looped out nucleotides in the three-dimensional structure. Cleavage reductions, not identified in the study on the longer B. subtilis RNA with the shorter PI helix 1 (e.g., nucleotides 22-26, 40-41, 113-116), become apparent when lysine is present in large excess over RNA. Nuclease VI cleavage enhancements within the P2a helix can be explained by an increase in the helix rigidity of the lysine-bound riboswitch and by the increased scission of the most accessible RNA regions that results from the hindrance of other cleavage sites upon lysine binding. Note that C79, G80 and G12 can be stacked in the free riboswitch, as suggested by the VI cleavages. Such stacking may contribute to the correct orientation of these nucleotides for interactions upon lysine binding.

In FIG. 41: A hypothetical model of lysine and K⁺ binding to the riboswitch junction. The lysine could enter the partially pre-formed binding pocket through an opening, which would otherwise be occupied in the bound state by K+ and G12. The lysine could then form specific hydrogen bonds with G114 and make multiple interactions that involve its £-ammonium group. The pocket could close via K+-mediated interactions and base-pairing of G12 and G11, that is next extended to the adjacent segment of the PI helix. Red lines designate important lysine-RNA interactions.

The P2-L4 tertiary contact seems to be more dynamic in character as reflected in the rather strong T2 (nucleotides 126-127, FIG. 27f) cleavage patterns in the lysine-free form, coupled with only weak protection on complex formation. The projection of the in-line cleavage reductions (this study and ref. 1) on the structure (FIG. 27g) provides the first glimpses into the primary determinants of lysine recognition and how the riboswitch junction folds on ligand binding. Assuming that helix P1 is not fully formed before lysine binding and that protections in P5 are, at least in part, due to P1-P5 interactions, Applicants propose that the main structural changes in solution, seen in both studies, involve stabilization of G80 and formation of the G12-C79 and G11NG163 junctional base pairs, followed by stabilization of the surrounding regions and the P1 helix (FIG. 271).

Prompted by pre-formation of tertiary riboswitch elements in solution, Applicants have crystallized the riboswitch in the absence of lysine. This 3.1 A ° structure (FIG. 42) is very similar to the lysine-bound form, except that it lacks lysine and junctional K⁺¹. Although the absence of the expression platform and long P1 helix facilitate formation of this conformation, the structure emphasizes the importance of RNA interactions in maintaining the riboswitch conformation, suggests a crucial role of K⁺¹ in mediation of lysine-RNA but not RNA-RNA interactions, and reinforces the feasibility of lysine stabilizing a largely preformed riboswitch structure. In FIG. 42: Lysine riboswitch structure in the free state. a, All-atom superposition of the lysine riboswitch structures from crystals grown in the presence (red) and absence (blue) of lysine. R.m.s.d. is 0.35 angstrom. b, Refined 3.1 A 2Fo-Fc electron density map (contoured at 1 a level) around the lysine-binding pocket in the lysine-free riboswitch structure. The view is similar to that of FIG. 44a. Both lysine and the K+ cation are missing in the structure.

In FIG. 44: Interpretation of certain regions of the electron density maps. a, Experimental electron density map (contoured at the 1 ( ) level) around the lysine binding pocket shown with the refined riboswitch model. The map was calculated using 2.4 A [Ir(NH3)6]3+ MAD data. b, 16 [Ir(NH3)6]3+ sites (2 sites are split) shown with the refined riboswitch model. c, Refined 1.9 A2Fo-Fc electron density map (contoured at the 1 ( ) level) around lysine and the K+ cation. Coordination bonds and distances are indicated. Water molecules are shown as pink spheres. d, Two Na+ cations interacting with the same 06 atom of guanine and sharing two water molecules. The map is as in (c).

The L box structure readily explains mutations that deregulate gene expression and confer resistance to AEC 27,28 (FIG. 43). In FIG. 43. Mutations in the sensing domain of the lysine riboswitch causing resistance to antibiotics and deregulation of the gene expression. Projection of known point mutations in the sensing domains of B. subtilis 5,27,38 and E. coli 28 lysine riboswitches on the sequence (a) and structure (b) of the T maritima lysine riboswitch. Mutations causing the derepression of riboswitches and resistance to AEC and L-4-oxalysine are indicated by pink arrows. Sequence conservation of the lysine riboswitch is indicated as in Supplementary FIG. 2. The point mutation A67C (B. subtilis numbering) in the kink-tum motif of B. subtilis riboswitch, mutations in the L2 and L3 100ps21, the long deletion of P2/J2-3/P327 and duplication within the PI helix 28 are not shown.

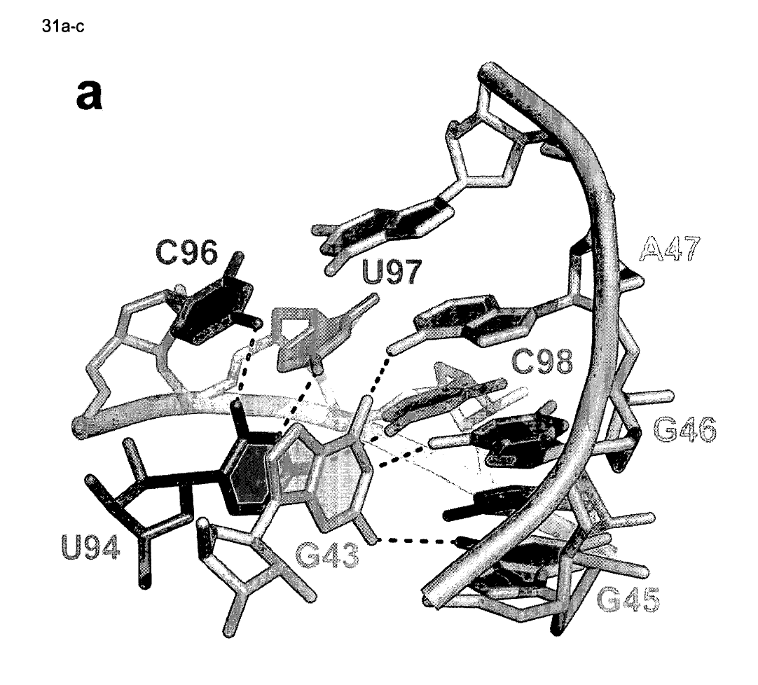

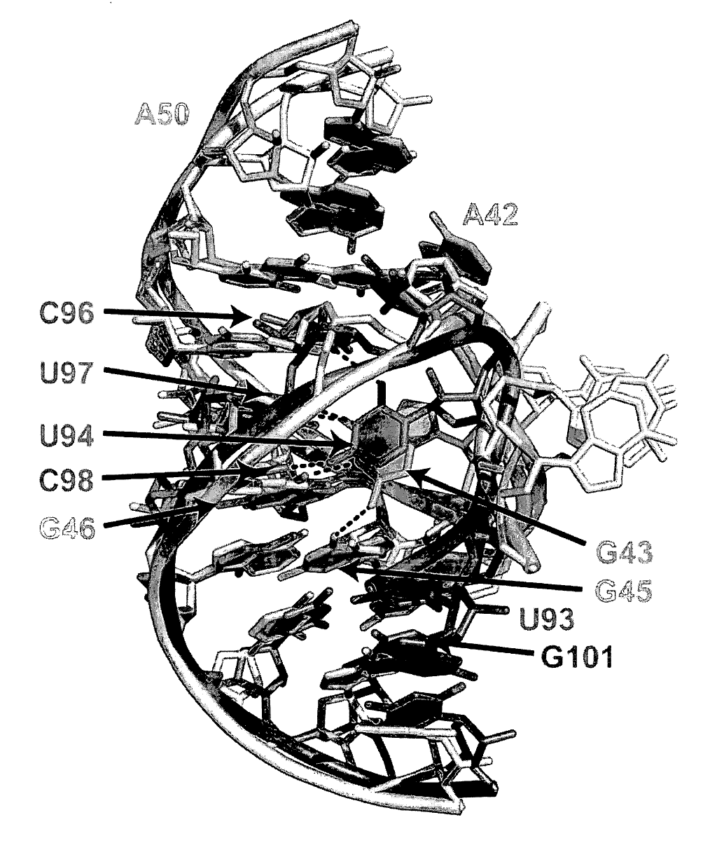

The G12A, G12C and G81A mutations disrupt the lysine-binding pocket, whereas the G11A, G11U, G9C and C166U substitutions prevent pairing of the P1 helix. Therefore, intracellular lysine and AEC cannot bind the mutated riboswitches, and the segment downstream of G161 engages in formation of an anti-terminator stem (FIG. 28), resulting in constitutive lysine production. In FIG. 28: Alternative conformations of the T. maritima lysine riboswitch and mechanism of lysine-induced transcription termination. a, The metabolite-sensing domain of the riboswitch folds in the presence of lysine and facilitates formation of the PI helix and a transcription terminator in the downstream expression platform. Transcription of the gene, therefore, is prematurely terminated. b, In the absence of lysine, the PI helix does not form and regions highlighted in green participate in the formation of an alternative anti-terminator hairpin conformation. In this case, transcription of the gene is not blocked. Nucleotides shown in italics have been added to the 265-nt RNA fragment used in primer extension experiments. Nucleotide numbering is consistent with the numbering used for the shorter RNA fragment.

The unusual architecture and high ligand specificity, achieved through a combination of shape complementarity and K⁺¹-assisted recognition of the bound lysine, distinguishes the lysine riboswitch from other riboswitches. Given the importance of lysine riboswitch controlled gene expression for bacterial viability and the absence of the diaminopimelate pathway in mammals, the structure provides critical details towards facilitating the design of lysine-like analogues targeting riboswitches and other cellular sites.

### EXAMPLES

The following examples are illustrative of the inventive subject matter and are not intended to be limitations thereon. When used and unless otherwise indicated, all percentages are based upon 100% by weight of the final composition.

### Example 1

### FMN Riboswitch Crystallization and Structure Determination

The complexes of the riboswitch with FMN and its analogues were prepared by mixing 0.4m mRNA with 0.7 mM ligand in a buffer containing 100 mM potassium acetate, pH 6.8, and 4 mM MgCl₂. FMN-riboswitch crystals were grown by hanging-drop vapor diffusion after mixing the complex with the reservoir solution (0.1M MES-sodium, pH 6.5, 100 mM MgCl₂ and 10% (w/v) PEG 4000) at equimolar ratio. For soaking, crystals were incubated for 10 h in the reservoir solution supplemented with 25% glycerol and the following heavy atom salts: 5 mM [Ir(NH3)6]Cl3, 15 mM MnCl2, 10 mM [Co(NH3)6]Cl3, 30 mM BaCl2 and 30 mM CsCl. Crystals were flash frozen in liquid nitrogen and data were collected at 100 K. The structure was determined using 3.0-A° multiwavelength anomalous dispersion (MAD) iridium data (See Table 1) and refined to Rwork/Rfree 20.0/24.3 with a native data set. FMN and cations were added to the model based on the analysis of 2Fo2Fc, Fo2Fc and anomalous electron density maps. Analogue-bound and metal-soaked riboswitch structures were refined using the native riboswitch model (See Table 2).

TABLE 1
Crystallography statistics for FMN structure

| | |
|---|---|
| Native | Native |
| (in vivo | (Annealing of |

| | | | |
|---|---|---|---|
| Crystal | Soaked in 5 mM [Ir(NH [symbol] ) [symbol] ]Cl₃ | transcription) | oligonucleotides) |

Data collection

| | | | | | |
|---|---|---|---|---|---|
| [symbol] | [symbol] | | | X29 | X29 |
| Space group | P3,21 | | | P3,21 | P3,21 |
| Cell dimension | | | | | |
| a, b, c(Å) | 71.5, 71.5, 138.7 | | | 71.5, 71.8, 141.1 | 71.5, 71.5, 140.6 |
| α, β, γ(°) | 90, 92, 120 | | | 90, 90, 120 | 90, 90, 120 |
| | Peak | Inflection | Remote | | |
| Wavelength (Å) | 1.10528 | 1.08591 | 1.10546 | 0.97969 | 1.01000 |
| Resolution [symbol] | [symbol] | [symbol] | [symbol] | [symbol] | [symbol] |
| R [symbol] | 9.9 (45.9) | 10.3 (36.0) | 9.2 (33.1) | 5.2 (57.0) | 6.1 (51.1) |
| [symbol] | 35.3 (3.1) | 39.9 ( [symbol] ) | 29.6 ( [symbol] ) | 67.0 ( [symbol] ) | 60.7 ( [symbol] ) |
| Completeness (%) [symbol] | 99.0 ( [symbol] ) | [symbol] ( [symbol] ) | 99.0 ( [symbol] ) | 99.0 ( [symbol] ) | 99.3 ( [symbol] ) |
| Unique reflections [symbol] | [symbol] (790) | 7,233 (629) | 7,145 (648) | 9,095 (879) | [symbol] ( [symbol] ) |
| Redundancy | 5.5 ( [symbol] ) | 9.0 (7.1) | 6.5 (5.4) | 16.3 (14.4) | 16.4 (15.8) |

Phasing

| | |
|---|---|
| Number of [ [symbol] ] [symbol] | 9 |
| Figure of [symbol] ( [symbol] ) | [symbol] |

Refinement (F > 0)

| | | | |
|---|---|---|---|
| Resolution (Å) | 20.0-3.00 | 20.01-2.95 | 20.00-3.05 |
| Number of reflections | | | |
| Working set | [symbol] | [symbol] | 7,487 |
| Test set | 400 | 431 | 382 |
| R [symbol] /R [symbol] (%) | [symbol] | [symbol] | [symbol] |
| Number of atoms | | | |
| RNA | 2,493 | 2,353 | 2,340 |
| FMN | 31 | 31 | 31 |
| Cations | 92 | 16 | 14 |
| Water | 1 | 2 | 2 |
| Average B-factors (Å [symbol] ) | | | |
| RNA | 111.4 | 66.9 | 63.4 |
| FMN | 75.2 | 54.2 | 57.8 |
| Cations | 833.1 | 77.1 | 81.9 |
| Water | 53.5 | 58.9 | 65.7 |
| R.m.s.d. from ideal geometry | | | |
| Bond lengths (Å) | 0.006 | 0.006 | 0.006 |
| Bond angles (°) | 0.989 | 1.007 | 1.025 |
| Estimated coordinate error [symbol] | 0.416 | 0.360 | 0.371 |

^{a}Values for the highest-resolution shell are in parentheses.
^{b}Estimated coordinate error based on maximum likelihood was calculated with REFMAC³¹. [symbol]
indicates data missing or illegible when filed

Analogue complexes and cation identification

| | | | | | | |
|---|---|---|---|---|---|---|
| Crystal | Riboflavin | Roseoflavin | Soaked in [symbol] | Soaked in [symbol] | Soaked in [symbol] | Soaked in [symbol] |

Data collection

| | | | | | | |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | | | | | | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

Refinement (F > 0)

| | | | | | | |
|---|---|---|---|---|---|---|
| [symbol] | [symbol] | [symbol] | [symbol] | [symbol] | [symbol] | [symbol] |
| [symbol] | | | | | | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | | | | | | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| Water | [symbol] | [symbol] | [symbol] | [symbol] | [symbol] | [symbol] |
| [symbol] | | | | | | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| Water | | [symbol] | | [symbol] | [symbol] | |
| [symbol] | | | | | | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

Values for the highest-resolution shell are in parentheses. [symbol]
Estimated coordinate error based on maximum likelihood was calculated with REFMAC [symbol] [symbol]
indicates data missing or illegible when filed

### Example 2

### Lysine Riboswitch Crystallization and Structure Determination

A 0.4 mM lysine riboswitch complex was prepared by mixing in vitro transcribed RNA and lysine in a buffer containing 100 mM potassium acetate, pH 6.8, and 4 mM MgCl₂. Crystals were grown by hanging-drop vapor diffusion after mixing the complex and the reservoir (18% (w/v) PEG4000, 100 mM sodium citrate, pH 5.7, and 20% isopropanol (v/v)) solutions at 1:1 ratio. For soaking, crystals were placed in the reservoir solution with PEG4000 replaced by 20% PEG400, and then incubated in the presence of either 3 mM [Ir(NH3)6]31 or 10-50 mM of Cs⁺¹, Ti⁺¹ and Mn⁺² salts for 7 h. Crystals were flash frozen in liquid nitrogen and data were collected at 100 K. The structure was determined using 2.4 A° multiwavelength anomalous dispersion iridium data and SHARP30. The RNA model was built using TURBO-FRODO (http://www.afmb.univ-mrs.fr/-TURBO-), and refined using the 1.9 A° native data set to Rwork/Rfree 19.2/22.9 (See Table 3 and FIG. 21). Lysine and cations were added to the model on the basis of analysis of 2Fo2Fc, Fo2Fc and anomalous electron density maps. Cations were modeled on the basis of the number of coordination bonds, their distances, coordination geometry and temperature factors. Analogue-bound and metal-soaked riboswitch structures were refined using the native riboswitch model (See Tables 4 and 5). Figures were prepared with PyMol.

TABLE 3
Lysine riboswitch crystallographic structure determination

| Crystal | Lysine-bound complex, soaked in [Ir(NH [symbol] ) [symbol] ] [symbol] ⁺ | | | Native, complex | Free state |
|---|---|---|---|---|---|
| Data collection | X29 | | | 24-ID-E | CuKa |
| Space group | P [symbol] | | | P [symbol] | P [symbol] |
| Cell dimensions | | | | | |
| a, b, c(Å) | 53.9, 78.0, 140.8 | | | 54.2, 79.0, 140.0 | 54.2, 78.6, 141.9 |
| α = β = γ(°) | 90.0 | | | 90.0 | 90.0 |
| | Peak | Inflection | Remove | | |
| Wavelength (Å) | 1.105199 | 1.105516 | 1.085799 | 0.9795 | 1.54 |
| Resolution (Å) | 20.00-2.40 (2.49-2.40) | 20.00-2.70 (2.80-2.70) | 20.00-2.60 (2.69-2.60) | 20.00-1.90 (1.97-1.90) | 20.00-3.10 (3.21-3.10) |
| R [symbol] | 11.3 (53.9) | 10.6 (60.0) | 9.5 (54.3) | 9.2 (59.5) | 19.7 (49.9) |
| I/σI | 23.9(3.4) | 20.2(2.9) | 15.3 (2.2) | 22.0 (8.6) | 7.5 (2.8) |
| Completeness (%) | 99.0 (97.6) | 99.4 (97.9) | 98.7 (96.9) | 96.1 (96.1) | 92.8 (89.5) |
| Unique reflections | 23.799 (2.282) | 17.201(1.653) | 18.598(1.794) | 46.081 (4.548) | 10.643 (1.005) |
| Redundancy | 6.5 (6.1) | 6.4 (6.0) | 4.4 (4.0) | 3.7 (3.9) | 4.3 (4.3) |

Phasing

| | |
|---|---|
| Number of [symbol] sites | 12 |
| Figure of merit | 0.47/0.24 |
| (acentric/centric) | |

Refinement (F > 0)

| | | | |
|---|---|---|---|
| Resolution (Å) | 20.0-2.40 | 20.00-1.90 | 20.00-3.10 |
| No. reflections | | | |
| Working set | 21.272 | 41.322 | 9.532 |
| Test set | 1.206 | 2.334 | |
| R [symbol] /R [symbol] | 20.4/24.3 | 19.2/22.9 | 18.8/22.8 |
| No. atoms | | | |
| RNA | 3.752 | 3.752 | 3.752 |
| Lysine | 10 | 10 | — |
| Cations | 141 | 37 | 11 |
| Water | 260 | 789 | 6 |
| B-factors | | | |
| RNA | 27.5 | 23.2 | 39.8 |
| Lysine | 6.0 | 7.8 | — |
| Cations | 53.7 | 26.5 | 31.9 |
| Water | 19.5 | 25.1 | 11.4 |
| R.m.s. deviations | | | |
| Bond lengths (Å) | 0.006 | 0.005 | 0.006 |
| Bond angles (°) | 1.193 | 1.124 | 1.228 |
| Estimated coord. error | 0.173 | 0.094 | 0.347 |

Values for the highest-resolution shell are in parentheses.
Estimated coordinate error based on maximum likelihood was calculated with REFMAC³¹.
No. of cations was calculated with Mg²⁺-coordinated water molecules [symbol]
indicates data missing or illegible when filed

TABLE 4
Crystallographic statistics for cation identification

| | Native | Native | Soaked in | Soaked in | Soaked in |
|---|---|---|---|---|---|
| Crystal | (anomalous K [symbol] ) | (No Mg²⁺) | 10 mM Tl-acetate | 10 mM CsCl | 50 mM MnCl₂ |
| Data collection | 24-ID-C | CiKa | X29 | 24-ID-C | 24-ID-C |
| Space group | P [symbol] | P [symbol] | P [symbol] | P [symbol] | P [symbol] |
| Cell dimensions | | | | | |
| a, b, c (Å) | 54.1, 78.9, 140.2 | 54.0, 78.8, 140.6 | 54.1, 78.8, 141.0 | 54.3, 70.0, 142.0 | 54.2, 78.7, 140.5 |
| α = β = γ(°) | 90.0 | 90.0 | 90.0 | 90.0 | 90.0 |
| Wavelength (Å) | 1.84 | 1.54 | 0.98 | 1.84 | 1.24 |
| Resolution (Å) | 20.00-2.90 (3.00-2.90) | 20.00-2.85 (2.95-2.85) | 20.00-2.50 (2.59-2.50) | 20.00-2.90 (3.00-2.90) | 20.00-2.70 (2.80-2.70) |
| R [symbol] | 11.8 (33.1) | 10.5 (49.8) | 10.2 (58.5) | 11.5 (26.9) | 9.5 (58.8) |
| I/σI | 12.4(2.5) | 15.7(2.7) | 26.8(4.1) | 11.7(2.7) | 21.8(2.8) |
| Completeness (%) | 97.5 (97.3) | 87.0 (89.2) | 99.2 (98.1) | 97.0 (96.8) | 96.6 (95.9) |
| Unique reflections | 13.523 (1.289) | 13.349 (1.328) | 21.350 (2.073) | 13.695 (1.356) | 16.392 (1.590) |
| Redundancy | 4.3 (3.6) | 3.8 (3.9) | 8.0 (7.5) | 4.5 (4.0) | 5.9 (5.6) |

Refinement (F > 0)

| | | | | | |
|---|---|---|---|---|---|
| Resolution (Å) | 20.00-2.90 | 20-2.85 | 20.00-2.50 | 20.00-2.90 | 20.00-2.70 |
| No. reflections | | | | | |
| Working set | 12.151 | 11.409 | 19.072 | 12.108 | 14.688 |
| Test set | 670 | 639 | 1.098 | 680 | 822 |
| R [symbol] /R [symbol] | 17.1/20.5 | 19.3/24.4 | 20.5/25.1 | 20.3/24.7 | 18.9/23.7 |
| No. atoms | | | | | |
| RNA | 3.752 | 3.752 | 3.752 | 3.752 | 3.756 |
| Lysine | 10 | 10 | 10 | 10 | 10 |
| Cations | 6 | 13 | 19 | 17 | 11 |
| Water | 18 | 10 | 118 | 26 | 37 |
| B-factors | | | | | |
| RNA | 34.3 | 45.4 | 28.7 | 32.8 | 35.1 |
| Lysine | 28.3 | 39.0 | 12.2 | 25.1 | 25.1 |
| Cations | 41.6 | 43.9 | 34.0 | 33.1 | 41.0 |
| Water | 26.1 | 25.2 | 19.5 | 23.9 | 27.6 |
| R.m.s deviations | | | | | |
| Bond lengths (Å) | 0.005 | 0.006 | 0.006 | 0.006 | 0.006 |
| Bond angles (°) | 1.259 | 1.328 | 1.293 | 1.311 | 1.239 |
| Coordinate error | 0.267 | 0.306 | 0.203 | 0.308 | 0.233 |

Values for the highest-resolution shell are in parentheses.
Estimated coordinate error based on maximum likelihood was calculated with REFMAC³¹. [symbol]
indicates data missing or illegible when filed

TABLE 5
Crystallography for the riboswitch-analog structures

| Crystal | AEC | L-4-Oxalysine | L-Homoarginine | IEL |
|---|---|---|---|---|
| Data collection | 24-ID-C | 24-ID-C | 24-ID-E | 24-ID-E |
| Space group | P [symbol] | P [symbol] | P [symbol] | P [symbol] |
| Cell dimensions | | | | |
| a, b, c (Å) | 54.4, 78.9, 142.9 | 54.5, 79.1, 142.5 | 53.8, 78.8, 140.1 | 54.8, 78.8, 143.3 |
| α = β = γ(°) | 90.0 | 90.0 | 90.0 | 90.0 |
| Wavelength (Å) | 1.9987 | 1.24 | 0.9795 | 0.9795 |
| Resolution (Å) | 20.00-2.80(2.90-2.80) | 20.00-2.50 (2.59-2.50) | 20.00-2.70 (2.80-2.70) | 20.00-2.90 (3.00-2.90) |
| R [symbol] | 11.2 (36.0) | 8.5 (49.7) | 11.9 (56.0) | 12.0(36.1) |
| I/σI | 21.4(2.5) | 27.8 (4.3) | 13.2(2.4) | 44.5(13.2) |
| Completeness (%) | 94.4 (62.8) | 97.7 (95.2) | 94.9 (89.0) | 94.3(82.5) |
| Unique reflections | 14.942 (97.6) | 21.717 (2.066) | 16.172 (1.479) | 13.631(1.159) |
| Redundancy | 5.2 (3.0) | 6.0 (5.9) | 4.7 (4.5) | 5.6(4.8) |

Refinement (F > 0)

| | | | | |
|---|---|---|---|---|
| Resolution (Å) | 20.00-2.80 | 20.00-2.50 | 20.00-2.70 | 20.00-2.90 |
| Number of reflections | | | | |
| Working set | 13.632 | 19.337 | 14.455 | 12.135 |
| Test set | 767 | 1.095 | 806 | 688 |
| R [symbol] /R [symbol] (%) | 19.7/22.5 | 20.2/26.8 | 20.2/24.1 | 17.8/21.1 |
| Number of atoms | | | | |
| RNA | 3.752 | 3.752 | 3.752 | 3.760 |
| Lysine analogs | 10 | 10 | 13 | 13 |
| Cations | 15 | 18 | 10 | 12 |
| water | 28 | 83 | 43 | 11 |
| B-factors | | | | |
| RNA | 23.1 | 32.3 | 25.2 | 30.2 |
| Lysine analogs | 25.2 | 16.3 | 11.1 | 23.0 |
| Cations | 33.5 | 34.9 | 22.5 | 37.2 |
| Water | 21.9 | 23.1 | 11.5 | 21.5 |
| R.m.s. deviations | | | | |
| Bond length (Å) | 0.007 | 0.006 | 0.005 | 0.007 |
| Bond angles (°) | 1.302 | 1.254 | 1.181 | 1.545 |
| Estimated coordinate error | 0.274 | 0.221 | 0.247 | 0.264 |

Values for the highest-resolution shell are in parentheses.
Estimated coordinate error based on maximum likelihood was calculated with REFMAC³¹. [symbol]
indicates data missing or illegible when filed

### Example 3

### RNA Preparation and Complex Formation

The 112-nucleotide sensing domain of the F. nucleatum FMN riboswitch followed by a hammerhead ribozyme was cloned into the pUT7 vector and transcribed in vitro using T7 RNA polymerase. The RNA was purified by denaturing polyacrylamide gel electrophoresis and anion-exchange chromatography. Alternatively, the riboswitch was formed by the annealing of two chemically synthesized RNAs (0.4 mM each):

(SEQ ID NO: 2)
GGAUCUUCGGGGCAGGGUGAAAUUCCCGACCGGUGGUAUAGUCCACGAA
AGCUU
(SEQ ID NO: 3)
GCUUUGAUUUGGUGAAAUUCCAAAACCGACAGUAGAGUCUGGAUGAGAG
AAGAUUC

designed to engineer crystal contacts, in the presence of FMN or its analogues in the binding buffer at 37° C. for 30 min followed by incubation on ice. The 143-nucleotide sensing domain of the B. subtilis FMN riboswitch, the 220-nucleotide full-length F. nucleatum FMN riboswitch and the 172-nucleotide fragment of the B. subtilis lysine riboswitch were transcribed and purified as above. Ligand concentrations were estimated spectrophotometrically using the extinction coefficients ε₄₅₀=12,500 M⁻¹ cm⁻¹ for FMN, ε₅₀₅=31,000 M⁻¹ cm⁻¹ for roseoflavin, and ε₄₄₅=12,500 M⁻¹ cm⁻¹ for riboflavin and lumiflavin.

### Example 4

### Crystallization and X-Ray Crystallography

Crystals were grown at 20° C. using hanging-drop vapor diffusion by mixing 1 ml complex with 1 ml reservoir solution. The FMN-bound complex produces crystals of the saturated yellow color, characteristic for the oxidized form of FMN, under several conditions. The best crystals were obtained in the solution containing 0.1M MES-sodium, pH 6.5, 100-200 mM MgCl₂ and 6-13% (w/v) PEG 4000 for 1-4 weeks. The crystals of the analogue-bound complexes grew in solution containing 0.1M Tris-HCl, pH 8.4, 200 mM MgCl₂ and 6-10% (w/v) PEG 4000 for about 1 week. The FMN-bound native and heavy-atom-soaked crystals were grown using the transcribed RNA. Although the analogue-bound crystals could grow using the transcribed RNA, resolution of the crystals was improved with the annealed riboswitch.

X-ray diffraction data were reduced using HKL2000 (HKL Research). The structure was determined using 3.0-A° MAD iridium data and autoSHARP (see, e.g., de La Fortelle, E. & Bricogne, G. in Methods in Enzymology 472-494, Academic Press, 1997). The RNA model was built using TURBO-FRODO (see http://www.afmb.univmrs.fr/-TURBO-) and refined with REFMAC (see, e.g., Murshudov, G. N., Vagin, A. A. & Dodson, E. J. Refinement of macromolecular structures by the maximum-likelihood method. Acta Crystallogr. D 53, 240-255 (1997)). The final 2.95-A° riboswitch model contained 109 nucleotides (nucleotides 54-56 were cleaved off during crystallization), one FMN molecule, two potassium and 14 magnesium cations. Mn2⁺, Cs⁺, Ba2⁺ and [Co(NH₂)₆]³⁺ cations were positioned based on the anomalous electron density maps (Supplementary FIGS. 7, 8, 10 and 11). Mn2⁺ and K⁺ cations were modelled according to location of their mimics, coordination geometry and distances (see, e.g., Feig, A. L. & Uhlenbeck, O. C. in The RNA World 2nd edn (eds Gesteland, R. F., Cech, T. R. & Atkins, J. F.) 287-319 (Cold Spring Harbor Laboratory Press, 1999).

### Example 5

### Fluorescence Measurements

Fluorescent assays were performed based on intrinsic fluorescence of FMN and its analogues, which become quenched after specific interaction of the ligands with the riboswitch fragment. In all assays, intensity of fluorescence emission was measured at 530 nM with excitation at 450 nM. Each experiment was performed about two to four times at room temperature using a Tecan M1000 fluorimeter.

For the binding affinity measurements, the fragments of the F. nucleatum and B. subtilis FMN riboswitches were titrated against 6×10⁻⁸ M FMN or 10⁻⁸ M analogues. RNA and ligands were premixed in 50 mM Tris-HCl, pH 7.4, 100 mM KCl and 2 mM MgCl₂ in the 96 half-area black flat plates for 10-60 min. The B. subtilis lysine riboswitch 27 was used as a negative control. After subtraction of the buffer fluorescence and normalization to the free ligand fluorescence, the data were fitted to equation (1):

$$F = 1 + ( f - 1 ) \frac{( {L}_{0} + {R}_{0} + {K}_{d} ) - \sqrt{{( {L}_{0} + {R}_{0} + {K}_{d} )}^{2} - 4 {L}_{0} {R}_{0}}}{2 {L}_{0}} \qquad ( 1 )$$

where F is normalized fluorescence intensity, L₀ and R₀ are the concentrations of ligand and RNA, K_{d} is the apparent dissociation constant, and f is a residual fluorescence intensity at the saturated concentration of ligand, determined by plotting F versus R₀.

For cation-dependence studies, the 112-nucleotide F. nucleatum FMN riboswitch was titrated against 6×10⁻⁸M of FMN in 50 mM Tris-HCl, pH 7.4, supplemented with 2 mM different cations (MgCl₂, MnCl₂, BaCl₂, CaCl₂, [Ir(NH₃)₆]Cl₃ and [Co(NH₃)₆]Cl₃) in the presence and absence of 100 mM KCl or in the presence of other monovalent cations (NaCl or CsCl). Binding affinities were determined using equation (1). The cation concentration required for the FMN binding was estimated by titration of different cations against a mixture of RNA (2×10⁻⁷ M) and ligand (6×10⁻⁸ M) in 50 mM Tris-HCl, pH 7.4. After background subtraction of the fluorescent quenching at each point in the absence of RNA, data were fitted to the Hill equation (2),

θ=[M]ⁿ/(K_{d}+[M]ⁿ) (2)

where h is the normalized FMN-bound fraction, n is Hill coefficient, [M] is the concentration of cation and K_{d} is the apparent dissociation constant. As the parameters K_{d} and n covary, the cation binding was roughly estimated as the ion concentration [M]_{1/2}=(K_{d})^{1/n} at which approximately 50% of FMN was bound to RNA.

### Example 6

### Footprinting Studies

For footprinting experiments (see, e.g., Serganov, A., Polonskaia, A., Ehresmann, B., Ehresmann, C. & Patel, D. J. Ribosomal protein S15 represses its own translation via adaptation of an rRNA-like fold within its mRNA. EMBO J. 8, 1898-1908, 2003), the 112-nucleotide F. nucleatum riboswitch was radioactively labelled at the 59 end by the kinase reaction. Samples (20 μl) of the radiolabelled RNA (100,000 c.p.m.) with a final RNA concentration 0.5 μM were preheated at 37° C. for 10 min in 50 mM Na-HEPES, pH 7.9, 50 mM KCl and 2 mM MgCl₂. Sixfold excess of FMN was added to the RNA and the mixtures were additionally incubated at 37° C. for 15 min. Cleavage reactions were performed with 0.003 U RNase V1 (Pierce) or 0.25 U RNase T2 (Sigma) at 37° C. for 10 min. Reactions were quenched by the addition of 80 μl cold buffer and were immediately extracted with phenol-chloroform and precipitated by ethanol. Radiolabelled pellets were dissolved and analysed by polyacrylamide gel electrophoresis.

### Example 7

### RNA Preparation and Complex Formation

The lysine riboswitch, followed by the hammerhead ribozyme, was transcribed in vitro using T7 RNA polymerase. RNA was purified by denaturing polyacrylamide gel electrophoresis (PAGE) and anion-exchange chromatography. Lysine analogues were added to RNA at a 2.5-2.75 to 1 molar ratio. To form a complex without Mg⁺², the RNA was mixed with 100 mM potassium-acetate, 1.0 mM EDTA and lysine. To prepare RNA for crystallization without lysine, 0.2 mm RNA was supplemented with 50 mM potassium acetate, pH 6.8, 50 mM sodium acetate, pH 6.9, and 2 mM MgCl2, and concentrated two fold by Speedvac. Before crystallization, sodium-citrate, pH 5.7, was added to the mixture up to 100 mM, and the RNA sample was heated at 55° C. for 5 min and cooled on ice for 15 min.

### Example 8

### Crystallization

Hanging drops were prepared by mixing 1 ml of the complex with 1 ml of the reservoir solution. The drops were equilibrated against 1 ml of reservoir solution at 20° C. for 1-2 weeks. The riboswitch in the free state was crystallized in the solution containing 21% (w/v) PEG4000, 100 mM Bis-Tris, pH 5.5, and 25% isopropanol (v/v). For cryoprotection, crystals were quickly passed through the stabilizing solution, which was the reservoir solution with PEG4000 replaced by 20% PEG400. For soaking, crystals were passed through several 5 ml drops of the stabilizing solution, and then incubated in 5 ml of stabilizing solution supplemented with 3 mM [Ir(NH3)6]Cl3, 10 mM CsCl, 10 mM thalium acetate, or 50 mM MnCl2 salts for 7-8 h.

### Example 9

### X-Ray Crystallography

Data were reduced using HKL2000 (HKL Research). The structure was determined using the autoSHARP option of SHARP and 2.4 A° MAD iridium data. The resulting experimental map was of excellent quality (FIG. 21a) for most of the RNA molecule. The structure contains 1 RNA and 16 iridium hexamine sites (2 of them are split) per asymmetric unit (FIG. 21b). The RNA model was built using 2.4 A ° MAD electron density map, and then refined with REFMAC31 using 1.9 A° native data. The bound lysine and several cations with octahedral coordination geometry were added on the basis of the 2Fo2Fc and Fo2Fc electron density maps. Cations were interpreted as Na⁺¹ or Mg⁺² on the basis of the coordination distances in the range of 2.25-2.85 A ° (FIGS. 21d) and 2.0-2.3 A°, respectively.

Water molecules were added using ARP/wARP32. K⁺¹ cations were added on the basis of the 1.9 A° 2Fo2Fc and 2.9 A° anomalous (FIG. 10) electron density maps and typical K⁺¹ coordination distances (FIG. 21c). The final 1.9 A° riboswitch model contains 174 nucleotides, 1 lysine molecule, 1 magnesium cation, 3 potassium and 29 sodium cations. Cs⁺¹, Ti⁺¹, and Mn⁺² cations were modelled on the basis of the anomalous maps (Supplementary FIGS. 7-9), whereas addition of other cations was guided by the high-resolution structure and the analysis of coordination geometries and distances.

### Example 10

### Primer Extension Assay

Primer extension experiments were performed using 265-nucleotide full-length riboswitch. The 32P-labelled 13-mer DNA oligonucleotide (100,000 c.p.m.), complementary to nucleotides 253-265 of RNA, was annealed to the 39 end of RNA (final RNA concentration 1 mM in the assay). Primer extension was conducted in 15 ml volume with 40 U of moloney murine leukaemia virus reverse transcriptase in 50 mM Na-HEPES, pH 7.9, 2 mM MgCl2, and variable concentrations of NaCl and KCl (FIG. 4), with or without a tenfold excess of lysine over RNA. After 30 min incubation at 37° C., the reactions were precipitated with ethanol, dissolved in loading buffer and analysed by 10% PAGE. Efficiency of lysine-induced pausing of reverse transcriptase at nucleotide A169 was quantified using FLA-7000 PhosphorImager and Image Gauge software (Fujifilm). Band intensities from independent experiments were averaged after gel-loading correction, background subtraction and normalization. The primer extension assay in the presence of lysine analogues was performed in 50 mM Na-HEPES, pH 7.9, 2 mM MgCl2, 50 mM KCl, and lysine analogues in the range 1028-1022 M. The data were fitted using a bimolecular equilibrium equation (Supplementary FIG. 12) and the resulting P50 values are reported in FIG. 4c. Reverse transcriptase sequencing reactions were run in parallel.

### Example 11

### Footprinting Studies

For footprinting experiments, the 174-nucleotide metabolite-sensing domain of the riboswitch was radioactively labelled at the 59 end by the kinase reaction. For in-line probing, 30,000-300,000 c.p.m. of 174-nucleotide RNA (1.6-16 nM) was incubated in 10-30 ml solution containing 50 mM Tris-HCl, pH 8.3, 100 mM KCl and 20 mM MgCl2 in the absence or presence of ,6-600-fold excess of lysine (FIG. 4) at room temperature for ,40 h. After incubation, aliquots were analysed by PAGE along with alkaline ladder and T1 nuclease digestion.

For nuclease footprinting experiments, 20 ml samples of 174-nucleotide RNA (100,000 c.p.m.) with a final RNA concentration 1 mM were preheated at 37° C. for 10 min in 50 mM Na-HEPES, pH 7.9, 50 mM KCl and 2 mM MgCl2. Mixtures were incubated with a tenfold excess of lysine over RNA at 37° C. for 15 min. Cleavage reactions were performed with 0.0025 U RNase V1 (Pierce) or 0.2 U RNase T2 (Sigma) at 37° C. for 10 min. Reactions were quenched by the addition of 80 ml cold buffer and were immediately extracted with phenolchloroform and precipitated with ethanol. Radiolabelled RNA products were dissolved and analysed by PAGE.

### Example 12

### Equilibrium Dialysis

The assay was performed as described in Sudarsan, et al., An mRNA structure in bacteria that controls gene expression by binding lysine, Genes Dev. 17:2688-2697 (2003) using 5 kDa DispoEquilibrium DIALYZERS (Harvard Apparatus). In brief, 30 ml RNA (from 0.001 to 20 mM) in the in-line probing buffer was placed in chamber A of the dialyser and equilibrated for 16 h at room temperature against chamber B containing 30 ml 3H-labelled lysine (1 nM; 6,000 c.p.m.) in the same buffer. The amount of bound lysine was calculated by subtracting the radioactivity counts of chamber B from chamber A. The data were fitted using a bimolecular equilibrium equation, assuming that the free lysine concentration is negligible.

Synthesis of Compounds of the Inventive Subject Matter

The compounds of the inventive subject matter may be readily prepared by standard techniques of organic chemistry and molecular biology.

In the preparation of the compounds of the inventive subject matter, one skilled in the art will understand that one may need to protect or block various reactive functionalities on the starting compounds or intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to “Protective Groups in Organic Chemistry,” McOmie, ed., Plenum Press, New York, N.Y.; and “Protective Groups in Organic Synthesis,” Greene, ed., John Wiley & Sons, New York, N.Y. (1981) for the teaching of protective groups which may be useful in the preparation of compounds of the inventive subject matter.

The product and intermediates may be isolated or purified using one or more standard purification techniques, including, for example, one or more of simple solvent evaporation, recrystallization, distillation, sublimation, filtration, chromatography, including thin-layer chromatography, HPLC (e.g. reverse phase HPLC), column chromatography, flash chromatography, radial chromatography, trituration, and the like.

### Tables 6-26

Tables 6-26 comprise a large data set, were consolidated into a separate part of the description, and were submitted in text format attached to this application on compact disk (CD) under PCT Rule 5.2(a). In Tables 6-26, the following abbreviations apply: FMN=flavin mononucleotide; IR6=iridium hexamine; BA2=barium cation; CS=cesium cation; MN2=manganese cation; CO6=cobalt hexamine; B2=riboflavin; RFN=roseoflavin; S2L=S-(2-aminoethyl)-L-cysteine; LYS=lysine; HOM=homoarginine; N61=N6-(1-iminoethyl)-L-lysine; KAD=potassium cation (anomalous data); MG2=magnesium cation; L4O=L-4-oxalysine; and TI=titanium cation.

### REFERENCES

The following literature references are believed to useful to an understanding of the inventive subject matter in the context of its place in the relevant art. Citation here is not to be construed as an assertion or admission that any reference cited is material to patentability of the inventive subject matter. Applicants will properly disclose information material to patentability in an Information Disclosure Statement. Each of the following documents is hereby incorporated by reference in its entirety, in this application.
Tucker B J, Breaker R R (2005). “Riboswitches as versatile gene control elements”. Curr Opin Struct Biol 15 (3): 342-8.
Vitreschak A G, Rodionov D A, Mironov A A, Gelfand M S (2004). “Riboswitches: the oldest mechanism for the regulation of gene expression?”. Trends Genet. 20 (1): 44-50.
Batey R T (2006). “Structures of regulatory elements in mRNAs”. Curr Opin Struct Biol 16 (3): 299-306.
a b Nahvi A, Sudarsan N, Ebert M S, Zou X, Brown K L, Breaker R R (2002). “Genetic control by a metabolite binding mRNA”. Chem Biol 9 (9): 1043.
Mironov A S, Gusarov I, Rafikov R, Lopez L E, Shatalin K, Kreneva R A, Perumov D A, Nudler E (2002). “Sensing small molecules by nascent RNA: a mechanism to control transcription in bacteria”. Cell 111 (5): 747-56.
Winkler W, Nahvi A, Breaker R R (2002). “Thiamine derivatives bind messenger RNAs directly to regulate bacterial gene expression”. Nature 419 (6910): 890-1.
Winkler W C, Cohen-Chalamish S, Breaker R R (2002). “An mRNA structure that controls gene expression by binding FMN”. Proc Natl Acad Sci USA 99 (25): 15908-13.
Sudarsan N, Barrick J E, Breaker R R. “Metabolite-binding RNA domains are present in the genes of eukaryotes”. RNA 9 (6): 644-7.
Cheah M T, Wachter A, Sudarsan N, Breaker R R (2007). “Control of alternative RNA splicing and gene expression by eukaryotic riboswitches”. Nature 447 (7143): 497-500.
Wachter A, Tunc-Ozdemir M, Grove B C, Green P J, Shintani D K, Breaker R R (2007). “Riboswitch control of gene expression in plants by splicing and alternative 3′ end processing of mRNAs”. Plant Cell 19 (11): 3437-50.
Bocobza S, Adato A, Mandel T, Shapira M, Nudler E, Aharoni A (2007). “Riboswitch-dependent gene regulation and its evolution in the plant kingdom”. Genes Dev. 21 (22): 2874-9.
Grundy F J, Henkin T M (1998). “The S box regulon: a new global transcription termination control system for methionine and cysteine biosynthesis genes in gram-positive bacteria”. Mol Microbiol 30 (4): 737-49.
Miranda-Rios J, Navarro M, Soberón M (2001). “A conserved RNA structure (thi box) is involved in regulation of thiamin biosynthetic gene expression in bacteria”. Proc Natl Acad Sci USA 98 (17): 9736-41.
Gelfand M S, Mironov A A, Jomantas J, Kozlov Y I, Perumov D A (1999). “A conserved RNA structure element involved in the regulation of bacterial riboflavin synthesis genes”. Trends Genet 15 (11): 439-42.
Barrick J E, Corbino Kans., Winkler W C, Nahvi A, Mandal M, Collins J, Lee M, Roth A, Sudarsan N, Jona I, Wickiser J K, Breaker R R (2004). “New RNA motifs suggest an expanded scope for riboswitches in bacterial genetic control”. Proc Natl Acad Sci USA 101 (17): 6421-6.
Corbino K A, Barrick J E, Lim J, Welz R, Tucker B J, Puskarz I, Mandal M, Rudnick N D, Breaker R R (2005). “Evidence for a second class of S-adenosylmethionine riboswitches and other regulatory RNA motifs in alpha-proteobacteria”. Genome Biol 6 (8): R70.
Weinberg Z, Barrick J E, Yao Z, Roth A, Kim J N, Gore J, Wang J X, Lee E R, Block K F, Sudarsan N, Neph S, Tompa M, Ruzzo W L, Breaker R R (2007). “Identification of 22 candidate structured RNAs in bacteria using the CM finder comparative genomics pipeline”. Nucleic Acids Res.
Blount K F, Breaker R R (2006). “Riboswitches as antibacterial drug targets”. Nat Biotechnol 24 (12): 1558-64.

The inventive subject matter being thus described, it will be obvious that the same may be modified or varied in many ways. Such modifications and variations are not to be regarded as a departure from the spirit and scope of the inventive subject matter and all such modifications and variations are intended to be included within the scope of the following claims.

Lengthy table referenced here
US20110237549A1-20110929-T00001
Please refer to the end of the specification for access instructions.

Lengthy table referenced here
US20110237549A1-20110929-T00002
Please refer to the end of the specification for access instructions.

Lengthy table referenced here
US20110237549A1-20110929-T00003
Please refer to the end of the specification for access instructions.

Lengthy table referenced here
US20110237549A1-20110929-T00004
Please refer to the end of the specification for access instructions.

Lengthy table referenced here
US20110237549A1-20110929-T00005
Please refer to the end of the specification for access instructions.

Lengthy table referenced here
US20110237549A1-20110929-T00006
Please refer to the end of the specification for access instructions.

Lengthy table referenced here
US20110237549A1-20110929-T00007
Please refer to the end of the specification for access instructions.

Lengthy table referenced here
US20110237549A1-20110929-T00008
Please refer to the end of the specification for access instructions.

Lengthy table referenced here
US20110237549A1-20110929-T00009
Please refer to the end of the specification for access instructions.

Lengthy table referenced here
US20110237549A1-20110929-T00010
Please refer to the end of the specification for access instructions.

Lengthy table referenced here
US20110237549A1-20110929-T00011
Please refer to the end of the specification for access instructions.

Lengthy table referenced here
US20110237549A1-20110929-T00012
Please refer to the end of the specification for access instructions.

Lengthy table referenced here
US20110237549A1-20110929-T00013
Please refer to the end of the specification for access instructions.

Lengthy table referenced here
US20110237549A1-20110929-T00014
Please refer to the end of the specification for access instructions.

Lengthy table referenced here
US20110237549A1-20110929-T00015
Please refer to the end of the specification for access instructions.

Lengthy table referenced here
US20110237549A1-20110929-T00016
Please refer to the end of the specification for access instructions.

Lengthy table referenced here
US20110237549A1-20110929-T00017
Please refer to the end of the specification for access instructions.

Lengthy table referenced here
US20110237549A1-20110929-T00018
Please refer to the end of the specification for access instructions.

Lengthy table referenced here
US20110237549A1-20110929-T00019
Please refer to the end of the specification for access instructions.

Lengthy table referenced here
US20110237549A1-20110929-T00020
Please refer to the end of the specification for access instructions.

Lengthy table referenced here
US20110237549A1-20110929-T00021
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110237549A1). An electronic copy of the table will also be available from the USPTO upon request and payment of the fee set forth in 37 CFR 1.19(b)(3).

1. A computer model of an FMN riboswitch generated from a data array comprising the atomic structure coordinates of an FMN riboswitch as set forth in any one of Tables 6-14, or a composite thereof.

2. A computer-readable storage medium encoded with the model of claim 1.

3. A computer comprising the model of claim 1, stored in memory.

4. The computer of claim 3, additionally comprising executable code for:
(a) displaying the data array as a 3-dimensional model;
(b) analyzing the binding site of the model of an FMN riboswitch;
(c) screening in silico a library for small molecules that fit into said binding site; and
(d) controlling a unit for assaying the small molecules determined in step (c) in a FMN riboswitch binding assay.

5. The computer model of claim 1, wherein the model is based upon a data array comprising atomic structure coordinates of a FMN riboswitch obtained in the presence of a sufficient amount of magnesium to saturate all sites for binding interactions between the FMN riboswitch and the magnesium.

6. A pharmacophore having a spatial arrangement of atoms defined by the binding pocket identified in the model of claim 1.

7. The pharmacophore of claim 6, wherein the spatial arrangement of atoms is determined in the presence of a sufficient amount of magnesium to saturate all sites for binding interactions between the FMN riboswitch and the magnesium.

8. An isolated compound, or a salt or solvate thereof, defined by the pharmacophore of claim 6.

9. A method for identifying a compound that interacts with a FMN riboswitch, utilizing a 3-D molecular model of a riboswitch as shown in any one of Tables 6-14, or a composite thereof, comprising:
(a) using the model in a method of rational drug design to identify candidate compounds that can bind a FMN riboswitch; and
(b) assaying the binding of a candidate compound identified in step (a) using a purified FMN riboswitch to thereby determine a binding characteristic, or lack thereof, of the compound.

10. The method of claim 9, wherein determining the binding characteristics of the compound in interaction with the riboswitch comprises determining a predicted minimum interaction energy, a predicted binding constant, a predicted dissociation constant, or a combination thereof, for the test compound in the model of the riboswitch.

11. The method of claim 9, wherein determining if the test compound interacts with the riboswitch comprises determining one or more predicted bonds, one or more predicted other interactions, or a combination thereof, for the test compound in the model of the riboswitch.

12. The method of claim 9, wherein the 3-D molecular model of a FMN riboswitch is determined from data obtained in the presence of a sufficient amount of magnesium to saturate all sites for binding interactions between the FMN riboswitch and the magnesium.

13. A method of killing bacteria, comprising contacting the bacteria with a compound identified by the method of claim 9.

14. A method for identifying a compound that interacts with a FMN riboswitch, utilizing the crystal structure of a FMN riboswitch, comprising the steps of:
(a) modeling the FMN riboswitch with a test compound; and
(b) determining if the test compound interacts with the FMN riboswitch.

15. A computer model of a lysine riboswitch generated from a data array comprising the atomic structure coordinates of a lysine riboswitch as set forth in any one of Tables 15-26, or a composite thereof.

16. A computer-readable storage medium encoded with the model of claim 15.

17. A computer comprising the model of claim 15, stored in memory.

18. The computer of claim 17, additionally comprising executable code for:
(a) displaying the data array as a 3-dimensional model;
(b) analyzing the binding site of the model of a lysine riboswitch;
(c) screening in silico a library for small molecules that fit into the binding site; and
(d) controlling a unit for assaying the small molecules determined in step (c) in a lysine riboswitch binding assay.

19. The computer model of claim 15, wherein the model is based upon a data array comprising atomic structure coordinates of a lysine riboswitch obtained in the presence of a sufficient amount of magnesium to saturate all sites for binding interactions between the lysine riboswitch and the magnesium.

20. A pharmacophore having a spatial arrangement of atoms defined by the binding pocket identified in the model o claim 15.

21. The pharmacophore of claim 20, wherein the spatial arrangement of atoms is determined in the presence of a sufficient amount of magnesium to saturate all sites for binding interactions between the lysine riboswitch and the magnesium.

22. An isolate compound, or a salt or solvate thereof, defined by the pharmacophore of claim 20.

23. A method for identifying a compound that interacts with a lysine riboswitch, utilizing a 3-D molecular model of a lysine riboswitch as shown in any one of Tables 15-26, or a composite thereof, comprising:
(a) using the model in a method of rational drug design to identify candidate compounds that can bind a lysine riboswitch; and
(b) assaying the binding of a candidate compound identified in step (a) using a purified lysine riboswitch to thereby determine a binding characteristic, or lack thereof, of the compound.

24. The method of claim 23, wherein determining the binding characteristics of the compound in interaction with the riboswitch comprises determining a predicted minimum interaction energy, a predicted binding constant, a predicted dissociation constant, or a combination thereof, for the test compound in the model of the riboswitch.

25. The method of claim 23, wherein determining if the test compound interacts with the riboswitch comprises determining one or more predicted bonds, one or more predicted other interactions, or a combination thereof, for the test compound in the model of the riboswitch.

26. The method of claim 23, wherein the 3-D molecular model of a lysine riboswitch is determined from data obtained in the presence of a sufficient amount of magnesium to saturate all sites for binding interactions between the lysine riboswitch and the magnesium.

27. A method of killing bacteria, comprising contacting the bacteria with a compound identified by the method of claim 23.

28. A method for identifying a compound that interacts with a lysine riboswitch, utilizing the crystal structure of a lysine riboswitch, comprising the steps of:
(a) modeling the lysine riboswitch with a test compound; and
(b) determining if the test compound interacts with the lysine riboswitch.
